(19) United States
(12) Patent Application Publication
(10) Pub. No.: US 2010/0047202 A1
(43) Pub. Date: Feb. 25, 2010

(54) Hair-Conditioning Agents Containing Selected Cationic Polymers and Water-Soluble Silicones

(75) Inventors: Dieter Goddinger, Klein Nordende (DE); Nicole Zuedel Fernandez, Hamburg (DE); Marlene Battermann, Asendorf (DE); Thomas Schroeder, Hamburg (DE)

Correspondence Address: Ratner Prestia, P.O. Box 980, Valley Forge, PA, 19482, US

(73) Assignee: Henkel AG & Co.KGaA, Duesseldorf (DE)

(21) Appl. No.: 12/488,894

(22) Filed: Jun. 22, 2009

Related U.S. Application Data
(63) Continuation of application No. PCT/EP2007/062292, filed on Nov. 13, 2007.

(30) Foreign Application Priority Data
Dec. 21, 2006 (DE) 10 2006 061 863.7

(51) Int. Cl. A61K 8/72; A61Q 5/12

(52) U.S. Cl. 424/70.12

(57) ABSTRACT

Cosmetic preparations, especially compositions that condition keratin fibers, which comprise at least two different cationic polymers and a water-soluble silicone. Said compositions are especially used to make the hair easy to comb and at the same time add volume to the hair. The compositions are preferably sprayed onto wet or dry keratin fibers, the composition preferably remaining on the keratin fibers until the next wash.

### CROSS-REFERENCE TO RELATED APPLICATIONS

This application is a continuation under 35 U.S.C. §§120 and 365(c) of International Application PCT/EP2007/062292, filed on Nov. 13, 2007. This application also claims priority under 35 U.S.C. §119 of DE 10 2006 061 863.7, filed on Dec. 21, 2006. The disclosures of PCT/EP2007/062292 and DE 10 2006 061 863.7 are incorporated herein by reference in their entirety.

### BACKGROUND OF THE INVENTION

The invention relates to hair-conditioning compositions containing at least two different cationic polymers and at least one water-soluble silicone and to the use of these compositions for cleansing and/or caring for skin and hair, in particular for conditioning hair.

The cosmetic treatment of skin and hair is an important part of human personal hygiene. Human hair is nowadays treated in many different ways with hair cosmetic preparations. Such treatments include for instance cleaning hair with shampoos, caring for and regenerating hair with rinses and masks and bleaching, dyeing and shaping hair with dyes, tints, permanent-wave compositions and styling preparations.

Due to the necessary content of oxidizing agents, not only oxidative methods such as for example bleaching or dyeing, but also oxidative fixing of the hair after the reductive stage of a cold wave have a harsh effect on the keratin-containing fibers. In some cases, lasting damage to the fibers may even occur. There has accordingly been no lack of attempts in the past to reduce the negative impact of oxidizing agents on the fibers. Not least due to the severe stresses placed on hair by such processes, but also due to cleaning the hair with shampoos and to environmental stresses, hair care products having an action which is as long-lasting as possible are becoming increasingly significant. Such hair care agents have an influence on the natural structure and properties of the hair. Accordingly, after such treatments, for example the hair's wet and dry combability and its hold and body may be optimized or the hair may be protected from increased splitting.

It has therefore long been usual to subject the hair to a special post-treatment. In this case, hair is conventionally treated with special active ingredients, for example quaternary ammonium salts, or special polymers in the form of a rinse. Depending on formulation, this treatment improves the hair's combability, hold and body and reduces splitting.

In recent times, “combined preparations” have been developed in order to reduce the complexity of conventional multistage methods, in particular for direct use by consumers.

In addition to the conventional components, for example for cleansing hair, these preparations also contain active ingredients which were previously found only in hair post-treatment agents. The consumer is thus saved one stage during use; at the same time packaging costs are reduced because one product less is used.

Available active ingredients both for separate post-treatment agents and for combined preparations in general preferably act on the surface of the hair. Active ingredients are thus known which impart gloss, hold, body, better wet or dry combability to the hair or prevent splitting. However, just as significant as the external appearance of hair is the internal structural cohesion of the hair fibers, which can be severely affected in particular in the case of oxidative and reductive processes such as dyeing and permanent waving.

Known active ingredients are, however, not capable of adequately meeting all requirements. A need accordingly still remains for active ingredients or active ingredient combinations for cosmetic agents which have good hair care properties and good biological degradability. In particular in formulations containing dyes or electrolytes, there is a need for additional hair care active ingredients which can be straightforwardly incorporated into known formulations.

Compatibility is another extremely important criterion for cosmetic compositions. Relevant dermatological journals report increasing levels of incompatibility among large parts of the population towards everyday products. These incompatibilities are the result, among other things, of changing consumer habits and the availability of highly exotic raw materials and foodstuffs in daily life. Consumers are therefore frequently advised to make use of indigenous products in their nutrition for example. One particularly major challenge facing the cosmetic chemist is thus to develop cosmetics for cleansing and caring for the skin and hair preferably on the basis of particularly compatible raw materials which are already tried and trusted. Particular attention is here paid to specially selected raw materials capable of performing multiple functions, so that a formulation contains the fewest possible constituents. The fewer constituents present in a formulation and the better these constituents are known, the lower is the risk of incompatibility. Should, exceptionally, a hypersensitivity reaction nevertheless occur, an allergist will very quickly be able to identify the irritant constituent for this consumer due to the small number of constituents. As a result, cosmetics overall become safer to use and more compatible for the consumer.

Active care ingredients which are sought are therefore in particular constituents which have already long been used cosmetics and are characterized by particularly good compatibility. At the same time, however, the spectrum of action and profile of properties of the cosmetic composition should not be disadvantageously modified in comparison with conventional compositions by the use of mild and particularly highly compatible constituents. This is furthermore complicated by its deliberately being desired to use the absolute minimum necessary number of constituents in the compositions according to the invention.

### DESCRIPTION OF THE INVENTION

The object of the present invention is therefore to provide compositions for agents for treating and conditioning keratin fibers, in particular for cleansing and caring for keratin fibers, which may very particularly preferably be used on keratin fibers following prior oxidative treatment. At the same time, the skin irritant action of these agents is reduced. Following agents for oxidative dyeing of hair, the color stability of the dyeing result is furthermore preferably distinctly improved despite the subsequent washing and conditioning process. In agents for carrying out cold waving, subsequent application of the compositions according to the invention likewise has a positive influence on both the volume and durability of the cold wave and the very good combability.

The objects are achieved according to the invention by a conditioning composition which contains already known constituents of cosmetic compositions as its active ingredients. The compositions according to the invention contain:

a) at least two different cationic polymers and
b) at least one water-soluble silicone.

The compositions according to the invention contain the cationic polymers and the water-soluble silicone in a specific quantity ratio. The weight ratio between the total quantity of cationic polymers and water-soluble silicones amounts to 20:1 to 1:20, more preferably 10:1 to 1:10, particularly preferably 5:1 to 1:5 and in particular 2.5:1 to 1:2.5 and very highly preferably 1.5:1 to 1:1.5. The two cationic polymers are present in a weight ratio relative to one another of 5:1 to 1:5, preferably of 3:1 to 1:3 and very particularly in a weight ratio of around 1:1.

The first essential constituent of the composition according to the invention comprises two different cationic polymers. The cationic polymers usable according to the invention are therefore described in detail below. The invention naturally also includes the recognition that, as a function of pH value, amphoteric polymers exhibit cationic properties. According to the invention, the term “cationic polymers” should thus also be taken to mean amphoteric polymers.

A feature common to these two groups of polymers is that they can potentially bear a cationic charge. Both cationic and amphoteric or zwitterionic polymers may therefore be characterized by their cationic charge density. The polymers according to the invention are distinguished by a charge density of at least 1 to 7 meq/g. A charge density of at least 2 to 7 meq/g is here preferred. A charge density of at least equal to 3 meq/g to 7 meq/g is particularly preferred.

Another characteristic feature of the polymers according to the invention is their molar mass. The molar mass of the particular polymer is taken to mean the molar mass stated by the manufacturer in the corresponding data sheets measured by the method therein. A molar mass of at least 50,000 g/u has proved particularly suitable according to the invention for selection of a suitable polymer. Polymers with a molar mass of more than 100,000 g/u have proved particularly suitable. Polymers with a molar mass of more than 1,000,000 g/u are very particularly suitable.

The deposition of polymers from surfactant solutions onto the surface of keratin fibers is an adsorption process. At present, this adsorption process is still not completely understood. In the prior art, suitable polymers are selected on the basis of the above-stated criteria of charge density or molar mass. These criteria are frequently not appropriate. From a physical standpoint, the deposition of polymers onto keratin fibers is an adsorption process.

All known adsorption equations in physical chemistry contain proportionality constants which correlate with the degree of surface coverage. However, without knowing the precise scientific background, the degree of surface coverage of keratin fibers could be associated with an adsorption probability of the polymers according to the invention. If the adsorption probability is defined as the product of cationic charge density and molar mass, this product may be utilized for targeted selection of suitable inventive polymers.

Suitable polymers exhibit a value for the product of the cationic charge density and the molar mass of greater than 100,000. Particularly suitable polymers are those which exhibit a value of at least 200,000 for this product.

Very particularly suitable polymers are those in which this product has a value of greater than 250,000. The most suitable polymers are those in which this product has a value of at least 1,000,000.

Better adsorption of cationic polymers according to the invention onto the surface of keratin fibers simultaneously leads to increased adsorption of the water-soluble silicones, which all in all leads to an improved finish of the surface of the keratin fibers and to easier combability of both the wet and the dry keratin fibers, elevated gloss, elevated volume etc. In simplified terms, modification of the surface may be imagined as follows: the cationic polymer or polymers is/are initially deposited onto the surface of the keratin fibers, which tends to be anionic. Such “bonds” are based on electrostatic attraction between what tends to be a negatively charged surface and the cationically charged polymers. Thereafter, the water-soluble silicones are attached, more generally by van der Waals interactions or H bridge-type bonds. The final effect is a smoothed, leveled surface which is distinguished by easy combability, elevated gloss, better stylability and thus a greater volume.

In the case of the present invention, it is probable that a mixed adsorbate film of the two cationic polymers is obtained on the surface of the keratin fibers. It may thus be advantageous to select cationic polymers which differ not only in structure, but also with regard to their molar mass and their cationic charge. On the other hand, mixed adsorption of polymers with excessive structural differences does not optimize the conditioning of keratin fibers. In unfavorable cases, this may instead even result in unwanted stressing of the keratin fibers. This is comparable with an excessively large quantity of only one cationic polymer.

It has surprisingly been found that using at least one natural polymer is distinctly advantageous. It is furthermore particularly advantageous for one of the two cationic polymers to be a natural polymer, said natural polymer being selected from among polysaccharides. Among the polysaccharides, the cationic derivatives of cellulose, starch, guar and/or chitosan have proved very particularly suitable according to the invention.

It has completely surprisingly been found that excellent results are achieved if both the cationic polymers are selected from the group of polysaccharides. Excellent results were here achieved when one of the two cationic polysaccharides was cationic chitosan. The best results were obtained by compositions comprising a cationic chitosan and a cationic guar derivative as cationic polymers. The cationic polymers are described in detail below.

Cationic polymers are polymers which comprise a group in the main and/or side chain which may be “temporarily” or “permanently” cationic. Polymers which are designated “permanently cationic” according to the invention are those which, irrespective of the pH value of the agent, comprise a cationic group. As a rule, these are polymers which contain a quaternary nitrogen atom, for example in the form of an ammonium group. Preferred cationic groups are quaternary ammonium groups. Polymers which have proven particularly suitable are those in which the quaternary ammonium group is bound via a C₁₋₄ hydrocarbon group to a main polymer chain synthesized from acrylic acid, methacrylic acid or the derivatives thereof.

Further cationic polymers according to the invention are “temporarily cationic” polymers. These polymers conventionally contain an amino group which at specific pH values assumes the form of a quaternary ammonium group and is thus cationic.

The cationic polymers according to the invention may not only be setting and/or film-forming and/or antistatic and/or softening polymers but also polymers with conditioning and/or thickening properties. Suitable cationically active polymers preferably comprise hair-setting and/or hair-conditioning polymers. Polymers should be taken to mean both natural and synthetic polymers, which may be cationically or amphoterically charged.

Preferred polymers are those which have sufficient solubility in water or alcohol in order, in the agent according to the invention, to pass completely into solution on application onto moist to wet hair. The cationic charge density preferably amounts 1 to 7 meq/g.

The cationic polymers may be homo- or copolymers, the quaternary nitrogen groups being present either in the polymer chain or preferably as substituents on one or more of the monomers. Monomers containing ammonium groups may be copolymerized with non-cationic monomers. Suitable cationic monomers are unsaturated, free-radically polymerizable compounds which bear at least one cationic group, in particular ammonium-substituted vinyl monomers such as for example trialkylmethacryloxyalkylammonium, trialkylacryloxyalkylammonium, dialkyldiallylammonium and quaternary vinylammonium monomers with cyclic, cationic nitrogen-containing groups such as pyridinium, imidazolinium or quaternary pyrrolidones, for example alkylvinylimidazolium, alkylvinylpyridinium or alkylvinylpyrrolidone salts. The alkyl groups of these monomers are preferably lower alkyl groups such as for example C₁ to C₇ alkyl groups, particularly preferably C₁— to C₃ alkyl groups.

Monomers containing ammonium groups may be copolymerized with non-cationic monomers. Suitable comonomers are for example acrylamide, methacrylamide; alkyl- and dialkylacrylamide, alkyl- and dialkylmethacrylamide, alkyl acrylate, alkyl methacrylate, vinylcaprolactone, vinylcaprolactam, vinylpyrrolidone, vinyl esters, for example vinyl acetate, vinyl alcohol, propylene glycol or ethylene glycol, the alkyl groups of these monomers preferably being C₁ to C₇ alkyl groups, particularly preferably C₁ to C₃ alkyl groups.

Suitable polymers with quaternary amine groups are for example the polymers described in the CTFA Cosmetic Ingredient Dictionary under the names polyquaternium, such as methylvinylimidazolium chloride/vinylpyrrolidone copolymer (Polyquaternium-16) or quaternized vinylpyrrolidone/dimethylaminoethyl methacrylate copolymer (Polyquaternium-11) and quaternary silicone polymers or oligomers such as for example silicone polymers with quaternary end groups (Quaternium-80).

One example of a suitable cationic polymer which may be present in the agent according to the invention is vinylpyrrolidone/dimethylaminoethyl-methacrylate methosulfate copolymer which is distributed by Gaf Co., USA under the tradenames Gafquat® 755 N and Gafquat® 734, with Gafquat® 734 being particularly preferred. Further cationic polymers are for example the copolymer of polyvinylpyrrolidone and imidazolinium methochloride distributed by BASF, Germany under the tradename Luviquat® HM 550, the terpolymer of dimethyldiallylammonium chloride, sodium acrylate and acrylamide distributed by Calgon/USA under the tradename Merquat® Plus 3300 and the vinylpyrrolidone/methacrylamidopropyltrimethylammonium chloride copolymer distributed by ISP under the tradename Gafquat® HS 100.

Homopolymers of the general formula (G1-I),

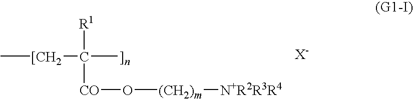

in which R¹ is —H or —CH₃, R², R³ and R⁴ are mutually independently selected from C₁₋₄ alkyl, alkenyl or hydroxyalkyl groups, m=1, 2, 3 or 4, n is a natural number and X⁻ a physiologically acceptable organic or inorganic anion, and copolymers substantially consisting of the monomer units listed in formula (G1-I) and nonionogenic monomer units, are particularly preferred cationic polymers. In the context of these polymers, those which are preferred according to the invention are those for which at least one of the following conditions applies:
R¹ denotes a methyl group
R², R³ and R⁴ denote methyl groups
m has the value 2.

Physiologically acceptable counterions X which may, for example, be considered are halide ions, sulfate ions, phosphate ions, methosulfate ions and organic ions such as lactate, citrate, tartrate and acetate ions. Halide ions, in particular chloride, are preferred.

One particularly suitable homopolymer is poly(methacryloyloxyethyltrimethylammonium chloride), which may if desired be crosslinked, with the INCI name Polyquaternium-37. Such products are commercially available for example under the names Rheocare® CTH (Cosmetic Rheologies) and Synthalen® CR (3V Sigma). Crosslinking may, if desired, proceed with the assistance of olefinically polyunsaturated compounds, for example divinylbenzene, tetraallyloxyethane, methylenebisacrylamide, diallyl ether, polyallyl polyglyceryl ether, or allyl ethers of sugars or sugar derivatives such as erythritol, pentaerythritol, arabitol, mannitol, sorbitol, sucrose or glucose. Methylenebisacrylamide is a preferred crosslinking agent.

The homopolymer is preferably used in the form of a nonaqueous polymer dispersion which should have a polymer fraction of no less than 30 wt. %. Such polymer dispersions are commercially available under the names Salcare® SC 95 (approx. 50% polymer fraction, further components: mineral oil (INCI name: Mineral Oil) and tridecyl-polyoxypropylene-polyoxyethylene ether (INCI name: PPG-1-Trideceth-6)) and Salcare® SC 96 (approx. 50% polymer fraction, further components: mixture of diesters of propylene glycol with a mixture of caprylic and capric acid (INCI name: Propylene Glycol Dicaprylate/Dicaprate) and tridecyl-polyoxypropylene-polyoxyethylene ether (INCI name: PPG-1-Trideceth-6)).

Copolymers with monomer units according to the formula (G1-I) preferably contain acrylamide, methacrylamide, acrylic acid C₁₋₄ alkyl esters and methacrylic acid C₁₋₄ alkyl esters as nonionogenic monomer units. Acrylamide is particularly preferred among these nonionogenic monomers. These copolymers, as described above for the homopolymers, may also be crosslinked. A copolymer which is preferred according to the invention is crosslinked acrylamide-methacryloyloxyethyltrimethylammonium chloride copolymer. Such copolymers, in which the monomers are present in a weight ratio of approx. 20:80, are commercially available as approx. 50% nonaqueous polymer dispersions under the name Salcare® SC 92.

Suitable cationic polymers, which are derived from natural polymers, are cationic derivatives of polysaccharides, for example cationic derivatives of cellulose, starch or guar. Chitosan and chitosan derivatives are furthermore suitable. Cationic polysaccharides have the general formula (G1-III)

G-O—B—N⁺RₐR_{b}R_{c}X⁻

G is an anhydroglucose residue, for example starch or cellulose anhydroglucose;
B is a divalent linking group, for example alkylene, oxyalkylene, polyoxyalkylene or hydroxyalkylene;
Rₐ, R_{B} and R_{c} are mutually independently alkyl, aryl, alkylaryl, arylalkyl, alkoxyalkyl or alkoxyaryl in each case having up to 18 C atoms, the total number of C atoms in Rₐ, R_{B} and R_{c} preferably amounting to at most 20;
X⁻ is a conventional counteranion and is preferably chloride.

A cationic cellulose is distributed by Amerchol under the name Polymer JR 400® and has the INCI name Polyquaternium-10. Another cationic cellulose has the INCI name Polyquaternium-24 and is distributed by Amerchol under the tradename Polymer LM-200. Further commercial products are the compounds Celquat® H 100, Celquat® and L 200. The stated commercial products are preferred cationic celluloses.

Suitable cationic guar derivatives are distributed under the tradename Jaguar® and have the INCI name Guar Hydroxypropyltrimonium Chloride. Particularly suitable cationic guar derivatives are furthermore also commercially available from Hercules under the name N-Hance®. Further cationic guar derivatives are distributed by Cognis under the name Cosmedia®. One preferred cationic guar derivative is the commercial product AquaCat® from Hercules. This raw material comprises an already predissolved cationic guar derivative.

Further particularly suitable cationic natural polymers are hydrocolloids of the chitosan type. From a chemical standpoint, these comprise partially deacetylated chitins of variable molecular weight which contain the, idealized, monomer building block (I):

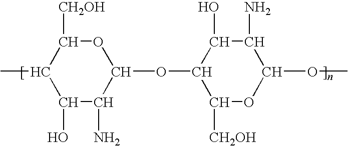

In contrast with most hydrocolloids, which are negatively charged in the range of biological pH values, chitosans are cationic biopolymers under these conditions. The positively charged chitosans are capable of interacting with oppositely charged surfaces and are therefore also used as film formers in cosmetic hair- and bodycare products. In addition to the chitosans, quaternized, alkylated and/or hydroxyalkylated derivatives, optionally also in microcrystalline form, may also be considered. Use may also proceed in the form of aqueous gels with a solids content in the range from 1 to 5 wt. %.

Chitosans are produced starting from chitin, preferably crustacean shell waste, which is available in large quantities as an inexpensive raw material. The chitin is here conventionally firstly deproteinated by addition of bases, demineralized by addition of mineral acids and finally deacetylated by addition of strong bases, the molecular weights possibly being distributed over a broad range.

In a preferred embodiment of the invention, particularly low-ash cationic biopolymers are used which are obtained by
(a) treating fresh crustacean shells with dilute aqueous mineral acid,
(b) treating the resultant demineralized first intermediate product with aqueous alkali metal hydroxide solution,
(c) treating the resultant slightly deproteinated second intermediate product again with dilute aqueous mineral acid,
(d) finally treating the resultant decalcified third intermediate product with concentrated aqueous alkali metal hydroxide solution and in so doing deacetylating down to a content of 0.05 to 0.5 and in particular 0.15 to 0.25 mol of acetamide per mol of monomer unit, and
(e) optionally carrying out a pressure/temperature post-treatment to adjust viscosity.

The chitosans to be used according to the invention comprise completely or partially deacetylated chitins. The molecular weights of the chitosan may be distributed over a broad range, for example from 20,000 to approx. 5 million g/mol. A low molecular weight chitosan with a molecular weight of 30,000 to 70,000 g/mol is for example suitable. Preferably, however, the molecular weight is above 100,000 g/mol, particularly preferably from 200,000 to 700,000 g/mol. The degree of deacetylation preferably amounts to 10 to 99%, particularly preferably 60 to 99%.

The chitosans or chitosan derivatives preferably assume neutralized or partially neutralized form. The degree of neutralization for the chitosan or the chitosan derivative is preferably at least 50%, particularly preferably between 70 and 100%, relative to the number of free base groups. Neutralizing agents which may in principle be used are any cosmetically compatible inorganic or organic acids, such as for example inter alia formic acid, tartaric acid, malic acid, lactic acid, citric acid, pyrrolidonecarboxylic acid, hydrochloric acid, with pyrrolidonecarboxylic acid being particularly preferred.

One suitable chitosan is for example distributed by Kyowa Oil & Fat, Japan, under the tradename Flonac®. It has a molecular weight of 300,000 to 700,000 g/mol and is 70 to 80% deacetylated. One preferred chitosan salt is chitosoniumpyrrolidone carboxylate, which is for example distributed by Amerchol, USA, under the name Kytamer® PC. The chitosan contained therein has a molecular weight of approx. 200,000 to 300,000 g/mol and is 70 to 85% deacetylated. Chitosan derivatives which may be considered are quaternized, alkylated or hydroxyalkylated derivatives, for example hydroxyethyl or hydroxybutyl chitosan. Further chitosan derivatives are readily commercially available under the tradenames Hydagen® CMF, Hydagen® HCMF and Chitolam® NB/101.

Further preferred cationic polymers are for example:
cationic alkyl polyglycosides according to DE-PS 44 13 686,
cationized honey, for example the commercial product Honeyquat® 50,
polymeric dimethyldiallylammonium salts and the copolymers thereof with esters and amides of acrylic acid and methacrylic acid. The products commercially available under the names Merquat® 100 (poly(dimethyldiallylammonium chloride)) and Merquat® 550 (dimethyldiallylammonium chloride/acrylamide copolymer) are examples of such cationic polymers,
copolymers of vinylpyrrolidone with quaternized derivatives of dialkylaminoalkyl acrylate and methacrylate, such as for example vinylpyrrolidone-dimethylaminoethyl methacrylate copolymers quaternized with diethyl sulfate. Such compounds are commercially available under the names Gafquat® 734 and Gafquat® 755,
vinylpyrrolidone-vinylimidazolium methochloride copolymers, as are offered for sale under the names Luviquat® FC 370, FC 550, FC 905 and HM 552,
quaternized polyvinyl alcohol, and the polymers known by the names Polyquaternium 2, Polyquaternium 17, Polyquaternium 18 and Polyquaternium 27 with quaternary nitrogen atoms in the polymer main chain,
vinylpyrrolidone/vinylcaprolactam/acrylate terpolymers, as are for example offered for sale with acrylic acid esters and acrylamides as the third monomeric building block under the name Aquaflex® SF 40.

The polymers known under the names Polyquaternium-24 (commercial product, for example Quatrisoft® LM 200) may also be used as cationic polymers. Copolymers of vinylpyrrolidone, as are available as commercial products Copolymer 845 (manufacturer: ISP), Gaffix® VC 713 (manufacturer: ISP), Gafquat® ASCP 1011, Gafquat® HS 110, Luviquat® 8155 and Luviquat® MS 370 may likewise be used according to the invention.

Further cationic polymers usable in the compositions according to the invention are “temporarily cationic” polymers. These polymers conventionally contain an amino group which at specific pH values assumes the form of a quaternary ammonium group and is thus cationic.

The cationic polymers are preferably present in the agents according to the invention in quantities of 0.05 to 10 wt. %, relative to the total agent. Quantities of 0.1 to 5 wt. % are particularly preferred.

Amphoteric polymers (G3) may furthermore be used according to the invention as cationic polymers. The term amphoteric polymers includes not only those polymers which contain in each molecule both free amino groups and free —COOH— or SO₃H groups and are capable of forming internal salts, but also zwitterionic polymers, which contain in each molecule quaternary ammonium groups and —COO⁻ or —SO₃⁻ groups, and those polymers which contain —COOH— or SO₃H groups and quaternary ammonium groups.

Like the cationic polymers, amphoteric polymers are very particularly preferred polymers.

One example of an amphoteric polymer usable according to the invention is the acrylic resin obtainable under the name Amphomer®, which is a copolymer of tert.-butylaminoethyl methacrylate, N-(1,1,3,3-tetramethylbutyl)acrylamide and two or more monomers from the group acrylic acid, methacrylic acid and the simple esters thereof.

Amphoteric polymers which are preferably used are those polymers which are substantially composed of
(a) monomers with quaternary ammonium groups of the general formula (G3-I),

R¹—CH═CR²—CO-Z-(CₙH₂ₙ)—N⁽⁺⁾R³R⁴R⁵A⁽⁻⁾ (G3-I)
in which R¹ and R² mutually independently denote hydrogen or a methyl group and R³, R⁴ and R⁵ mutually independently denote alkyl groups having 1 to 4 carbon atoms, Z is an NH group or an oxygen atom, n is an integer from 2 to 5 and A⁽⁻⁾ is the anion of an organic or inorganic acid, and
(b) monomeric carboxylic acids of the general formula (G3-II),

R⁶—CH═CR⁷—COOH (G3-II)
in which R⁶ and R⁷ are mutually independently hydrogen or methyl groups.

These compounds may be used according to the invention both directly and in salt form, which is obtained by neutralization of the polymers, for example with an alkali metal hydroxide. Very particularly preferred polymers are those in which monomers of type (a) are used, in which R³, R⁴ and R⁵ are methyl groups, Z is an NH group and A⁽⁻⁾ is a halide, methoxysulfate or ethoxysulfate ion; acrylamidopropyltrimethylammonium chloride is a particularly preferred monomer (a). Acrylic acid is preferably used as monomer (b) for the stated polymers.

Suitable starting monomers are for example dimethylaminoethylacrylamide, dimethylaminoethylmethacrylamide, dimethylaminopropylacrylamide, dimethylaminopropylmethacrylamide and diethylaminoethylacrylamide, if Z means an NH group, or dimethylaminoethyl acrylate, dimethylaminoethyl methacrylate and diethylaminoethyl acrylate, if Z is an oxygen atom.

The monomers containing a tertiary amino group are then quaternized in known manner, particularly suitable alkylating reagents being methyl chloride, dimethyl sulfate or diethyl sulfate. The quaternization reaction may proceed in aqueous solution or in solvent.

Those monomers of the formula (G3-I) which are derivatives of acrylamide or methacrylamide are advantageously used. Those monomers containing halide, methoxysulfate or ethoxysulfate ions as counterions are furthermore preferred. Those monomers of the formula (G3-I) in which R³, R⁴ and R⁵ are methyl groups are likewise preferred.

Acrylamidopropyltrimethylammonium chloride is one very particularly preferred monomer of the formula (G3-I).

Suitable monomeric carboxylic acids of the formula (G3-II) are acrylic acid, methacrylic acid, crotonic acid and 2-methylcrotonic acid. Acrylic or methacrylic acid, in particular acrylic acid, are preferably used.

Zwitterionic polymers usable according to the invention are produced from monomers of the formulae (G3-I) and (G3-II) using per se known polymerization methods. Further details regarding the polymerization method may be obtained from the relevant specialist literature.

Polymers which have proved particularly active are those in which the monomers of the formula (G3-I) were present in excess relative to the monomers of the formula (G3-II). It is therefore preferred according to the invention to use such polymers which consist of monomers of the formula (G3-I) and monomers of the formula (G3-II) in a molar ratio of 60:40 to 95:5, in particular of 75:25 to 95:5.

The amphoteric polymers are contained in the agents according to the invention preferably in quantities of from 0.05 to 10 wt. %, relative to the total agent. Quantities of 0.1 to 5 wt. % are particularly preferred.

Further amphoteric polymers usable according to the invention are the compounds stated in British published patent application 2 104 091, European published patent application 47 714, European published patent application 217 274, European published patent application 283 817 and German published patent application 28 17 369. Further suitable zwitterionic polymers are methacryloyl ethyl betaine/methacrylate copolymers, which are commercially available under the name Amersette® (Amerchol).

Water-soluble silicones may be mentioned as a further essential constituent of the present composition. Silicones are conventionally sparingly soluble to insoluble in water. Silicones rendered water-soluble by suitable substitution are, however, available. The term “water-soluble” here also includes “water-dispersible”. Silicones dispersed in water are therefore likewise included according to the invention. Solubility or dispersibility of silicones may for example be achieved by ethoxylating or propoxylating silicones. According to the invention, the term “water-soluble silicones” does not include dimethicones and/or dimethiconols. Water-soluble silicones can easily be recognized from their INCI name. The prefix PEG- or PPG- generally precedes the actual INCI name. Commercial products with the INCI names such as for example PEG-12 Dimethicone or PEG-Amodimethicone are typical representatives of water-soluble silicones. These are described in greater detail below.

Dimethicone copolyols (S3) form one group of preferred water-soluble silicones. Dimethicone copolyols may be illustrated by the following structural formulae:

(SiR¹₃)—O—(SiR²₂—O—)ₓ—(SiRPE-O—)_{y}—(SiR¹₃) (S3-I)

or by the following structural formula:

PE-(SiR¹₂)—O—(SiR²₂—O—)ₓ—(SiR¹₂)-PE (S3-II)

Branched dimethicone copolyols may be illustrated by the structural formula (S3-III):

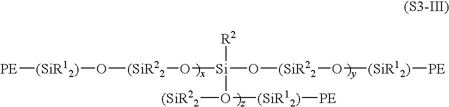

or by the structural formula (S3-IV):

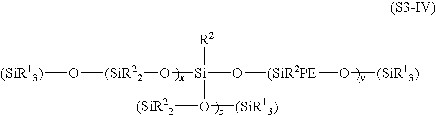

The residues R¹ and R² mutually independently denote in each case hydrogen, a methyl residue, a C₂ to C₃₀ linear, saturated or unsaturated hydrocarbon residue, a phenyl residue and/or an aryl residue. Non-limiting examples of the residues represented by R¹ and R² include alkyl residues, such as methyl, ethyl, propyl, isopropyl, butyl, isobutyl, pentyl, isopentyl, neopentyl, amyl, isoamyl, hexyl, isohexyl and the like; alkenyl residues, such as vinyl, halovinyl, alkylvinyl, allyl, haloallyl, alkylallyl; cycloalkyl residues, such as cyclobutyl, cyclopentyl, cyclohexyl and the like; phenyl residues, benzyl residues, halogenated hydrocarbon residues, such as 3-chloropropyl, 4-bromobutyl, 3,3,3-trifluoropropyl, chlorocyclohexyl, bromophenyl, chlorophenyl and the like and sulfur-containing residues, such as mercaptoethyl, mercaptopropyl, mercaptohexyl, mercaptophenyl and the like; R¹ and R² are preferably an alkyl residue containing 1 to approx. 6 carbon atoms, and R¹ and R² are most preferably methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂ CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—; and —(CH₂)₃C(O)SCH₂CH₂—. Preferably R¹ and R² are methyl, phenyl and C₂ to C₂₂ alkyl residues. The C₂ to C₂₂ alkyl residues are very particularly preferably lauryl, stearyl and behenyl residues. PE denotes a polyoxyalkylene residue. Preferred polyoxyalkylene residues are derived from ethylene oxide, propylene oxide and glycerol. The numbers x, y and z are integers and run in each case mutually independently from 0 to 50,000. The molar weights of the dimethicone copolyols are between 1000 D and 10000000 D. Viscosities are between 100 and 10000000 cPs measured at 25° C. with the assistance of a glass capillary viscometer using the Dow Corning Corporate Test Method CTM 0004 of 20 Jul. 1970. Preferred viscosities are between 1000 and 5000000 cPs, very particularly preferred viscosities are between 10000 and 3000000 cPs. The most preferred range is between 50000 and 2000000 cPs.

Of course, the teaching according to the invention also provides that the dimethicone copolymers may also be present as an emulsion. In this case, the corresponding dimethicone copolyol emulsion may be produced both after the production of the corresponding dimethicone copolyols from the latter and using the conventional methods of emulsification known to a person skilled in the art. To this end, any of cationic, anionic, nonionic or zwitterionic surfactants and emulsifiers may be used as auxiliary materials for producing the corresponding emulsions. It goes without saying that the dimethicone copolyol emulsions may also be produced directly by an emulsion polymerization method. Such methods are also well known to a person skilled in the art. In this respect, reference is made for example to the “Encyclopedia of Polymer Science and Engineering”, Volume 15, Second Edition, pages 204 to 308, John Wiley & Sons., Inc. 1989. Reference is explicitly made to this standard work.

If the dimethicone copolyols according to the invention are used in the form of an emulsion, the droplet size of the emulsified particles then amounts according to the invention to 0.01 μm to 10000 μm, preferably 0.01 to 100 μm, very particularly preferably 0.01 to 20 μm and most preferably 0.01 to 10 μm. Particle size is here determined using the light scattering method.

If branched dimethicone copolyols are used, it should be understood that the branching is greater in this case than the chance branching which arises due to impurities in the respective monomers. For the purposes of the present compound, branched dimethicone copolyols should therefore be taken to mean that the degree of branching is greater than 0.01%. Preferably, the degree of branching is greater than 0.1% and very particularly preferably greater than 0.5%. The degree of branching is determined in this case from the ratio of unbranched monomers, i.e. the quantity of monofunctional siloxane, to the branched monomers, i.e. the quantity of tri- and tetrafunctional siloxanes. According to the invention, dimethicone copolyols with both a low and a high degree of branching may be very particularly preferred.

The dimethicone copolyols (S3) are in the compositions according to the invention in quantities of 0.01 to 10 wt. %, preferably 0.01 to 8 wt. %, particularly preferably 0.1 to 7.5 wt. % and in particular 0.1 to 5 wt. % of dimethicone copolyol relative to the composition.

It is also possible according to the invention for the dimethicone copolyols to form their own phase in the compositions according to the invention. In this case, the quantity of dimethicone copolyol may amount to up to 40 wt. %, preferably in quantities of up to 25 wt. % relative to the total composition.

Amino-functional silicones or amodimethicones (S4) are silicones which comprise at least one (optionally substituted) amino group.

Such silicones may, for example, be described by the formula (S4-I)

M(RₐQ_{b}SiO_{(4-a-b)/2)x}(R_{c}SiO_{(4-c)/2)y}M (S4-I)

wherein in the above formula R is a hydrocarbon or a hydrocarbon residue with 1 to approx. 6 carbon atoms, Q is a polar residue of the general formula —R¹HZ, in which R¹ is a divalent linking group, which is attached to hydrogen and the residue Z, composed of carbon and hydrogen atoms, carbon, hydrogen and oxygen atoms or carbon, hydrogen and nitrogen atoms, and Z is an organic, amino-functional residue, which contains at least one amino-functional group; “a” assumes values in the range from approx. 0 to approx. 2, “b” assumes values in the range from approx. 1 to approx. 3, “a”+“b” is less than or equal to 3, and “c” is a number in the range from approx. 1 to approx. 3, and x is a number in the range from 1 to approx. 2,000, preferably from approx. 3 to approx. 50 and most preferably from approx. 3 to approx. 25, and y is a number in the range from approx. 20 to approx. 10,000, preferably from approx. 125 to approx. 10,000 and most preferably from approx. 150 to approx. 1,000, and M is a suitable silicone end group, as known in the prior art, preferably trimethylsiloxy. Non-limiting examples of the residues represented by R include alkyl residues, such as methyl, ethyl, propyl, isopropyl, isopropyl, butyl, isobutyl, amyl, isoamyl, hexyl, isohexyl and the like; alkenyl residues, such as vinyl, halovinyl, alkylvinyl, allyl, haloallyl, alkylallyl; cycloalkyl residues, such as cyclobutyl, cyclopentyl, cyclohexyl and the like; phenyl residues, benzyl residues, halogenated hydrocarbon residues, such as 3-chloropropyl, 4-bromobutyl, 3,3,3-trifluoropropyl, chlorocyclohexyl, bromophenyl, chlorophenyl and the like and sulfur-containing residues, such as mercaptoethyl, mercaptopropyl, mercaptohexyl, mercaptophenyl and the like; R is preferably an alkyl residue containing 1 to approx. 6 carbon atoms, and R is most preferably methyl. Examples of R¹ include methylene, ethylene, propylene, hexamethylene, decamethylene, CH₂CH(CH₃)CH₂—, phenylene, naphthylene, —CH₂CH₂SCH₂CH₂—, —CH₂CH₂OCH₂—, —OCH₂CH₂—, —OCH₂ CH₂CH₂—, —CH₂CH(CH₃)C(O)OCH₂—, —(CH₂)₃ CC(O)OCH₂CH₂—, —C₆H₄C₆H₄—, —C₆H₄CH₂C₆H₄—; and —(CH₂)₃C(O)SCH₂CH₂—.

Z is an organic, amino-functional residue containing at least one functional amino group. A possible formula for Z is NH(CH₂)_{z}NH₂, in which z is 1 or more. Another possible formula for Z is —NH(CH₂)_{z}(CH₂)_{zz}NH, in which both z and zz are mutually independently 1 or more, this structure comprising diamino ring structures, such as piperazinyl. Z is most preferably an —NHCH₂CH₂NH₂ residue. Another possible formula for Z is —N(CH₂)_{z}(CH₂)_{zz}NX₂ or —NX₂, in which each X of X₂ is independently selected from the group consisting of hydrogen and alkyl groups having 1 to 12 carbon atoms, and zz is 0.

Q is most preferably a polar, amino-functional residue of the formula CH₂CH₂CH₂NHCH₂CH₂NH₂. In the formulae, “a” assumes values in the range from 0 to approx. 2, “b” assumes values in the range from approx. 2 to approx. 3, “a”+“b” is less than or equal to 3, and “c” is a number in the range from approx. 1 to approx. 3. The molar ratio of the RₐQ_{b} SiO_{(4-a-b)/2} units to the R_{c}SiO_{(4-c)/2} units is in the range from approx 1:2 to 1:65, preferably from approx. 1:5 to approx. 1:65 and most preferably from approx. 1:15 to approx. 1:20. If one or more silicones of the above formula are used, then the various variable substituents in the above formula may be different in the various silicone components which are present in the silicone mixture.

Preferred agents according to the invention are characterized in that they contain an amino-functional silicone of the formula (S4-II)

R′ₐG₃₋ₐ-Si(OSiG₂)ₙ-(OSiG_{b}R′_{2-b})ₘ—O—SiG₃₋ₐ-R′ₐ (S4-II),

in which:
G is —H, a phenyl group, —OH, —O—CH₃, —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃;
a denotes a number between 0 and 3, in particular 0;
b denotes a number between 0 and 1, in particular 1,
m and n are numbers, the sum of which (m+n) amounts to between 1 and 2000, preferably between 50 and 150, with n preferably assuming values from 0 to 1999 and in particular from 49 to 149 and m preferably assuming values from 1 to 2000, in particular from 1 to 10,
R′ is a monovalent residue selected from

### —N(R″)—CH₂—CH₂—N(R″)₂

### —N(R″)₂

### —N⁺(R″)₃ A⁻

### —N⁺H(R″)₂ A⁻

### —N⁺H₂(R″) A⁻

—N(R″)—CH₂—CH₂—N⁺R″H₂ A⁻, each R″ denoting identical or different residues from the group —H, -phenyl, -benzyl, C₁₋₂₀ alkyl residues, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A represents an anion which is preferably selected from chloride, bromide, iodide or methosulfate.

Particularly preferred agents according to the invention are characterized in that they contain an amino-functional silicone of the formula (S4-III)

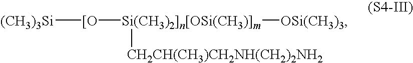

in which m and n are numbers, the sum of which (m+n) amounts to between 1 and 2000, preferably between 50 and 150, with n preferably assuming values from 0 to 1999 and in particular from 49 to 149 and m preferably assuming values from 1 to 2000, in particular from 1 to 10.

These silicones are denoted in accordance with the INCI Declaration as Trimethylsilylamodimethicones.

Particularly preferred agents according to the invention are also those which are characterized in that they contain an amino-functional silicone of the formula (S4-IV)

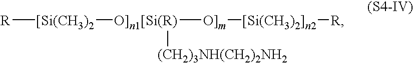

in which R denotes —OH, —O—CH₃ or a —CH₃ group and m, n1 and n2 are numbers the sum of which (m+n1+n2) amounts to between 1 and 2000, preferably between 50 and 150, the sum (n1+n2) preferably assuming values from 0 to 1999 and in particular from 49 to 149 and m preferably assuming values from 1 to 2000, in particular from 1 to 10.

Very particularly preferred agents according to the invention are characterized in that they contain an amino-functional silicone of the formula (S4-V)

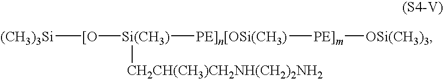

in which m and n are numbers, the sum of which (m+n) amounts to between 1 and 2000, preferably between 50 and 150, with n preferably assuming values from 0 to 1999 and in particular from 49 to 149 and m preferably assuming values from 1 to 2000, in particular from 1 to 10 and PE furthermore mutually independently denoting a polyalkylene oxide block, providing that at least one polyalkylene oxide block must be present. The number of the polyalkylene oxide blocks amounts in each case to between 1 and 5000, preferably 1 and 3000 and particularly preferably from 1 to 1000, with 1 to 100 repeat units being very highly preferred. In the simplest case, the polyalkylene oxide is formed of ethylene oxide or propylene oxide or butylene oxide or mixtures thereof or a mixture of ethylene oxide and propylene oxide. It is preferred to use ethylene oxide.

These silicones are denoted in accordance with the INCI Declaration as PEG-Trimethylsilylamodimethicones.

Particularly preferred agents according to the invention are also those which are characterized in that they contain an amino-functional silicone of the formula (S4-VI)

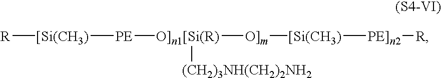

in which R denotes —OH, —O—CH₃ or a —CH₃ group and m, n1 and n2 are numbers the sum of which (m+n1+n2) amounts to between 1 and 2000, preferably between 50 and 150, the sum (n1+n2) preferably assuming values from 0 to 1999 and in particular from 49 to 149 and m preferably assuming values from 1 to 2000, in particular from 1 to 10 and furthermore PE mutually independently denoting a polyalkylene oxide block, providing that at least one polyalkylene oxide block must be present. The number of the polyalkylene oxide blocks amounts in each case to between 1 and 5000, preferably 1 and 3000 and particularly preferably from 1 to 1000, with 1 to 100 repeat units being very highly preferred. In the simplest case, the polyalkylene oxide is formed of ethylene oxide or propylene oxide or butylene oxide or mixtures thereof or a mixture of ethylene oxide and propylene oxide. It is preferred to use ethylene oxide.

These silicones are denoted in accordance with the INCI Declaration as PEG-Amodimethicones.

Irrespective of which amino-functional silicones are used, the aminated silicones containing PE groups being particularly preferred, preferred compositions according to the invention are those in which the amino-functional silicone has an amine value of above 0.25 meq/g, preferably of above 0.3 meq/g and in particular of above 0.4 meq/g. The amine value here denotes the milliequivalents of amine per gram of the amino-functional silicone. It can be determined by titration and may also be stated in the unit mg of KOH/g.

The amodimethicones (S4) are in the compositions according to the invention in quantities of 0.01 to 10 wt. %, preferably 0.01 to 8 wt. %, particularly preferably 0.1 to 7.5 wt. % and in particular 0.1 to 5 wt. % of amodimethicone relative to the composition.

It is also possible according to the invention for the amodimethicones to form their own phase in the compositions according to the invention. In this case it may be appropriate for the composition to be briefly homogenized by shaking immediately before application. In this case, the quantity of amodimethicone may amount to up to 40 wt. %, preferably in quantities of up to 25 wt. % relative to the total composition.

Only just recently, completely novel polyammonium/polysiloxane compounds have become known, in which the siloxane substructures are optionally joined together via ammonium substructures. Such compounds and the use thereof in cosmetics are described, for example, in published patent application WO 02/10257.

The compositions according to the invention may contain as silicon at least one polyammonium/polysiloxane compound which is of the structure described below. The polyammonium/polysiloxane compounds contain:

a1) at least one polyalkylene oxide structural unit of the general formulae:

-A-E-, -E-A-, -A-E-A′- and/or -A′-E-A-, in which:

A denotes one of the groups: —CH₂C(O)O—, —CH₂CH₂C(O)O—, —CH₂CH₂CH₂C(O)O—, —OC(O)CH₂—, —OC(O)CH₂CH₂— and/or —OC(O)CH₂CH₂CH₂—,
A′ means: —CH₂C(O)—, —CH₂CH₂C(O)—, —CH₂CH₂CH₂C(O)—, —C(O)CH₂—, —C(O)CH₂CH₂— and/or —C(O)CH₂CH₂CH₂— and
E denotes a polyalkylene oxide group of the general formulae:

—[CH₂CH₂O]_{q}—[CH₂CH(CH₃)O]ᵣ and/or —[OCH(CH₃)CH₂]ᵣ,—[OCH₂CH₂]_{q}—,

with q=1 to 200 and r=0 to 200, the terminal oxygen atom of group A bonding to the terminal —CH₂ group of group E, and the terminal carbonyl carbon atom of group A′ bonding to the terminal oxygen atom of group E, in each case forming ester groups, and/or at least one terminal polyalkylene oxide structural unit of the formula -A-E-R², in which A and E have the above-stated meaning, and R² denotes H, straight-chain, cyclic or branched C₁-C₂₀ hydrocarbon residue which may be interrupted by —O—, or —C(O)— and substituted with —OH, and be acetylenic, olefinic or aromatic,
a2) at least one divalent or trivalent organic residue which contains at least one ammonium group,
a3) at least one polysiloxane structural unit of the general formula:

—K—S—K—,

in which S denotes —Si(R¹)₂—O[—Si(R¹)₂—O]ₙ—Si(R¹)₂— and
in which R¹ denotes C₁-C₂₂ alkyl, C₁-C₂₂ fluoroalkyl or aryl, n denotes 0 to 1000, and, if two or more groups S are present in the polysiloxane compound, these may be identical or different,
in which K represents a divalent or trivalent straight-chain, cyclic or branched C₂-C₄₀ hydrocarbon residue which may be interrupted by —O—, —N—, —NR¹—, —C(O)—, —C(S)—, —N⁺(R³)— and —N⁺(R¹)(R³)— and substituted with —OH,
in which R¹ is defined as above, or optionally represents a bond to a divalent residue R³, and
in which R³ represents a monovalent or divalent straight-chain, cyclic or branched C₁-C₂₀ hydrocarbon residue which may be interrupted by —O—, —NH—, —C(O)—, —C(S)— and substituted with —OH, or -A-E-R², in which A, E and R are defined as above,
the residues K possibly being identical or different from one another, and, in the case that K represents a trivalent residue, the third valence is saturated by a bond to the above-stated organic residue which contains at least one ammonium group,
a4) an organic or inorganic acid residue for neutralizing the charges arising from the ammonium group(s).

The polysiloxane compounds according to the invention are characterized in that they comprise the above-defined components al) to a4). The polysiloxane compounds are here formed by attaching the stated structural units or residues al) to a3) to one another. Component a4) serves to neutralize the positive charges arising from component a2).

The polysiloxane compounds according to the invention may be oligomeric or polymeric compounds. Oligomeric compounds here also include the case described below, in which the polysiloxane compound comprises just one repeat unit.

Polymeric polysiloxane compounds according to the invention here arise naturally by alternating linkage of divalent residues.

In the case of the polymeric polysiloxane compounds according to the invention, the terminal atomic groups arise from the terminal atomic groups of the starting materials used. This is known per se to a person skilled in the art.

In a preferred embodiment, the polymeric polysiloxane compounds according to the invention are linear polyammonium/polysiloxane compounds which are composed of structural components a1) to a3). The linear polymeric polysiloxane compounds according to the invention, in particular their linear polymeric main chain formed from the repeat units, may accordingly be synthesized by alternately stringing together polyalkylene oxide structural units a1), organic residues which contain at least one, preferably quaternary, ammonium group a2) and polysiloxane structural units a3). This means that any further free valances optionally present in the structural components (as may occur in trivalent residues as component a2) or in trivalent residues K) preferably do not serve to form polymeric side chains or polymeric branches.

In a further embodiment, the main chain of the linear polymeric polysiloxane compounds according to the invention is made up of the organic residues, which contain at least one ammonium group a2), and the polysiloxane structural units a3), and the polyalkylene oxide structural units al) bond as side chains to the trivalent organic ammonium group residue. The following structures may, for example, be obtained:

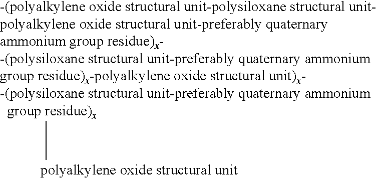

Depending on the molar ratio of the monomeric starting compounds, polysiloxane compounds according to the invention which comprise just one repeat unit may be obtained. This is known per se to a person skilled in the art. This case gives rise, for example, to polysiloxane compounds according to the invention of the structure:
(terminal polyalkylene oxide structural unit-quaternary ammonium group residue-polysiloxane structural unit-quaternary ammonium group residue-terminal polyalkylene oxide structural unit).

The polysiloxane compounds according to the invention preferably substantially consist of components al) to a4), the polymeric polysiloxane compounds according to the invention naturally comprising the terminal groups which result from the reaction of the monomeric starting materials. Monofunctional chain terminators may, however, also be used.

The polyalkylene oxide structural units a) may comprise divalent residues of the general formulae:

-A-E-, -E-A-, -A-E-A′- and/or -A′-E-A-.

Residue A here means:
—CH₂C(O)O—, —CH₂CH₂C(O)O—, —CH₂CH₂CH₂C(O)O—, —OC(O)CH₂—, —OC(O)CH₂CH₂— and/or —OC(O)CH₂CH₂CH₂—
Residue A′ here means:
—CH₂C(O)—, —CH₂CH₂C(O)—, —CH₂CH₂CH₂C(O)—, —C(O)CH₂—, —C(O)CH₂CH₂— and/or —C(O)CH₂CH₂CH₂—.
The polyalkylene oxide group E of the general formulae:
—[CH₂CH₂O]_{q}—[CH₂CH(CH₃)O]ᵣ and/or —[OCH(CH₃)CH₂]ᵣ[OCH₂CH₂]_{q} with q=1 or 2 to 200 and r=0 to 200, here include all possible ethylene oxide/propylene oxide groupings. Random ethylene oxide/propylene oxide copolymer groups or ethylene oxide/propylene oxide block copolymer groups with any desired arrangement of one or more ethylene oxide or propylene oxide blocks may accordingly be involved.

Residues A or A′ are attached to group E in such a manner that the terminal oxygen atom of group A bonds to the terminal —CH₂ group of group E, and the terminal carbonyl carbon atom of group A′ bonds to the terminal oxygen atom of group E, in each case forming ester groups.

The polyalkylene oxide structural units al) may furthermore comprise monovalent, i.e. terminal polyalkylene oxide structural units of the formula -A-E-R², in which A and E have the above-stated meaning, and R² denotes H, straight-chain, cyclic or branched C₁-C₂₀ hydrocarbon residue which may be interrupted by —O—, or —C(O)— and substituted with —OH, and be acetylenic, olefinic or aromatic.

The component a2) of which the polysiloxane compounds according to the invention are composed is at least one divalent or trivalent organic residue which contains at least one ammonium group. The residue is preferably bonded to the other components of the polysiloxane compounds according to the invention via the nitrogen atom of one or more ammonium groups in the organic residue. The term “divalent” or “trivalent” means that the organic ammonium residue comprises two or three free valences for forming bonds in particular to the other components of the polysiloxane compounds according to the invention. The ammonium residue is conveniently represented by an NH₄⁺ group in which at least two hydrogen atoms are substituted by organic groups. It preferably comprises a secondary or quaternary, particularly preferably a quaternary, ammonium group. According to a general definition (cf. for example Römpp-Chemie-Lexikon), a quaternary ammonium group is a group in which all four hydrogen atoms of an NH₄⁺ group are replaced by organic residues.

Component a2) of the polysiloxane compounds according to the invention is at least one polysiloxane structural unit of the general formula:

—K—S—K—,

S therein is a polysiloxane group of the general formula

—Si(R¹)₂—O[—Si(R¹)₂—O]ₙ—Si(R¹)₂—,

in which R¹ means C₁-C₂₂ alkyl, C₁-C₂₂ fluoroalkyl or aryl, preferably phenyl, n=0 to 1000, and, if two or more groups S are present in the polysiloxane compound, these may be identical or different.

R¹ is preferably C₁-C₁₈ alkyl, C₁-C₁₈ fluoroalkyl and aryl. R¹ is furthermore preferably C₁-C₁₈ alkyl, C₁-C₆ fluoroalkyl and aryl. R¹ is furthermore preferably C₁-C₆ alkyl, C₁-C₆ fluoroalkyl, more preferably C₁-C₄ fluoroalkyl, and phenyl. R¹ is still more preferably methyl, ethyl, trifluoropropyl and phenyl.

The term “C₁-C₂₂ alkyl” means for the purposes of the present invention that the aliphatic hydrocarbon groups have 1 to 22 carbon atoms, which may be straight-chain or branched. Methyl, ethyl, propyl, n-butyl, pentyl, hexyl, heptyl, nonyl, decyl, undecyl, iso-propyl, neopentyl, and 1,2,3-trimethylhexyl may be listed by way of example.

The term “C₁-C₂₂ fluoroalkyl” means for the purposes of the present invention aliphatic hydrocarbon compounds with 1 to 22 carbon atoms which may be straight-chain or branched and are substituted with at least one fluorine atom. Monofluoromethyl, monofluoroethyl, 1,1,1-trifluoroethyl, perfluoroethyl, 1,1,1-trifluoropropyl, 1,2,2-trifluorobutyl may be listed by way of example.

The term “aryl” means for the purposes of the present invention phenyl which is unsubstituted or mono- or polysubstituted with OH, F, Cl, CF₃, C₁-C₆ alkyl, C₁-C₆ alkoxy, C₃-C₇ cycloalkyl, C₂-C₆ alkenyl or phenyl. The expression may optionally also mean naphthyl.

K represents a divalent or trivalent straight-chain, cyclic or branched C₂-C₄₀ hydrocarbon residue which may be interrupted by —O—, —NH—, —N—, C(O)—, —C(S)—, —N⁺(R³)—, —NR¹—, and —N⁺(R¹)(R³)— and substituted with —OH.

“Interrupted” here means that, in the case of the divalent residues, a —CH₂ grouping and, in the case of the trivalent residues, a —CH grouping of the hydrocarbon residues is replaced by the stated groups. This also applies to the remainder of the description when this term is used.

Group K bonds via a carbon atom to the silicon atom of group S.

Group K may, as seen above, likewise preferably comprise quaternary ammonium groups, such that, in addition to the ammonium groups in the stated component a2), ammonium groups are obtained in the polysiloxane compounds according to the invention.

The polysiloxane compounds according to the invention may, as for example in residue K, comprise amino groups. Reacting the polysiloxane compounds according to the invention with acids results in the protonation thereof. Such polysiloxane compounds comprising protonated amino groups are included in the scope of the present invention.

Bonding of component a3), the polysiloxane structural unit —K—S—K—, to the other structural components via residue K preferably does not proceed via a nitrogen atom of residue K.

R¹ is defined as above or optionally represents a bond to a divalent residue R³, such that a cycle is obtained.

R³ represents a monovalent or divalent straight-chain, cyclic or branched C₁-C₂₀ hydrocarbon residue which may be interrupted by —O—, —NH—, —C(O)—, —C(S)— and substituted with —OH, or -A-E-R², in which A, E and R² are defined as above.

The residues K may be identical or different from one another and, in the case that K represents a trivalent residue, the third valence is saturated by a bond to the above-stated organic residue which contains at least one ammonium group.

The polysiloxane compounds according to the invention furthermore contain component a4), at least one organic or inorganic anionic acid residue for neutralizing the charges arising from the ammonium group(s). Organic or inorganic acid residues are residues which formally arise from the elimination of one or more protons from organic or inorganic acids and include, for example, halides, such as fluoride, chloride, bromide, sulfates, nitrates, phosphates, carboxylates, such as formate, acetate, propionate etc., sulfonates, sulfates, polyether carboxylates and polyether sulfates etc. Chloride is preferred. The organic or inorganic anionic acid residues as component a4) of the polysiloxane compounds according to the invention may be identical or different from one another. Accordingly, the reaction of amines with alkyl halides preferably gives rise to halide ions, while for example carboxylates are obtained from the carboxylic acids which may be added on reaction of bisepoxides with amines.

In a preferred embodiment of the polysiloxane compounds according to the invention, K represents a divalent or trivalent straight-chain, cyclic or branched C₂-C₄₀ hydrocarbon residue which may be interrupted by —O—, —NH—, —N—, —NR¹—, —C(O)—, —C(S)— and substituted with —OH, in which R¹ is defined as above, and wherein the residues K may be identical or different from one another.

The above-stated organic residue which contains at least one preferably quaternary ammonium group is preferably a residue of the general formula:

—N¹—F—N¹—,

in which N¹ is a quaternary ammonium group of the general formula —(R⁴)N⁺(R⁵)—, in which R⁴ represents a monovalent or divalent straight-chain, cyclic or branched C₁-C₂₀ hydrocarbon residue which may be interrupted by —O—, —NH—, —C(O)—, —C(S)— and substituted with —OH, and R⁵ represents a monovalent straight-chain, cyclic or branched C₁-C₂₀ hydrocarbon residue which may be interrupted by —O—, —NH—, —C(O)—, —C(S)— and substituted with —OH, or a single bond to a divalent residue R⁴ or a tetravalent residue F, and the residues R⁴ and R⁵ within the group —N¹—F—N¹— and in der polysiloxane compound may be identical or different from one another,
F is a divalent or tetravalent straight-chain, cyclic or branched C₂-C₃₀ hydrocarbon residue which may be interrupted by —O—, —NH—, —N—, —C(O)—, —C(S)—, a siloxane chain S, the above-stated relationships applying for S, and substituted with —OH.

With regard to further details of the definitions of the quaternary ammonium group of the formula —N¹—F—N¹— (preferred embodiments etc.), reference is made to the explanations of the first embodiment of the present invention for component a, the polyammonium/polysiloxane compounds, in which this group is embodied, and which are also valid in this more general context.

The above-stated organic residue which contains at least one, preferably quaternary, ammonium group may furthermore preferably be a residue of the general formula

—(R⁶)N⁺(R⁷)—,

in which R⁶ is a monovalent or divalent straight-chain, cyclic or branched C₁-C₃₀ hydrocarbon residue which may be interrupted by —O—, —NH—, —C(O)—, —C(S)— and substituted with —OH, or R⁶ represents a single bond to a trivalent residue K.

R⁷ is a monovalent straight-chain, cyclic or branched C₁-C₂₀ hydrocarbon residue which may be interrupted by —O—, —NH— —C(O)—, —C(S)— and substituted with —OH, or -A-E-R², in which -A-E-R² has the above-stated meaning, or a single bond to a divalent residue R⁶ or to a trivalent residue K.

The residues R⁶ and R⁷ may be identical or different from one another.

With regard to further details of the definitions of the quaternary ammonium group of the formula —(R⁶)N⁺(R⁷)— (preferred embodiments), reference is made to the explanations of the second, third and fourth embodiments regarding polyammonium/polysiloxane compounds, constituent a), of the present active ingredient complex according to the invention, in which this group is embodied, and which are also valid in this more general context.

The above-stated organic residue which contains at least one ammonium group may furthermore preferably be a residue of the general formula:

—N⁵—F¹—N⁵—,

in which N⁵ is an ammonium group of the general formula

—(R²³)N⁺(R²⁴)—, in which

R²³ represents hydrogen, a monovalent or divalent straight-chain, cyclic or branched C₁-C₂₀ hydrocarbon residue which may be interrupted by —O—, —NH—, —C(O)—, —C(S)— and substituted with —OH,
R²⁴ represents hydrogen, a monovalent straight-chain, cyclic or branched C₁-C₂₀ hydrocarbon residue which may be interrupted by —O—, —NH—, —C(O)—, —C(S) and substituted with —OH, or represents a single bond to a divalent residue R²³, and the residues R²³ and R²⁴ within the group —N⁵—F¹—N⁵— and in the polysiloxane compound may be identical or different from one another,
F¹ means a divalent straight-chain, cyclic or branched —N-hydrocarbon residue which may be interrupted by —O—, —NH—, —C(O)—, —N—, —C(S)— or by a group -E-, and in which a plurality of groups N⁵ and F¹ may in each case be identical or different from one another.

With regard to further details of the definitions of the ammonium group of the formula —N⁵—F¹—N⁵— (preferred embodiments), reference is made to the explanations of the fifth embodiment in relation to component a, the polyammonium/polysiloxane compounds of the present invention, in which this group is embodied by way of example, and which are also valid in this more general context.

Components a) of the active ingredient complex according to the invention, the polyammonium/polysiloxane compounds, are described below in greater detail with reference to five preferred embodiments of these compounds.

One particular embodiment of the polyammonium/polysiloxane compounds (hereinafter designated the first embodiment of component a) of the active ingredient complex), in which the above-stated organic residue, which contains at least one preferably quaternary ammonium group as component a2) of the polysiloxane compounds according to the invention, represents a residue of the general formula:

—N¹—F—N¹—

is represented by the polysiloxane compounds of the following general formula (I):

—[B—N¹—F—N¹]ₘ— (I)

in which m=2 to 500,
B means -A-E-K—S—K-E-A- and is additionally optionally -A-E-A′- or -A′-E-A-, in which S, K, -A-E-, -E-A-, -A-E-A′- or -A′-E-A- and —N¹—F—N¹— are defined as above, and the fraction of group -A-E-A′- or -A′-E-A- in group B may be selected such that the mass of -A-E-A′- or -A′-E-A- ranges from 0 to 90%, preferably 0% or 0.1 to 50% of the mass of the polysiloxane fraction S in the polymer.

The first embodiment of the polyammonium/polysiloxane compounds preferably relates to linear alkylene oxide-modified polyquaternary polysiloxanes of the general formula (I′),

—[B—N¹—F—N¹],— (I′)

in which m represents 2 to 500,
B -A-E-K—S—K-E-A-,
S —Si(R¹)₂—O[Si(R¹)₂—O]ₙ—Si(R¹)₂—
R¹ represents C₁-C₂₂ alkyl, C₁-C₂₂ fluoroalkyl or aryl,
n represents 0 to 1000,
K represents a divalent straight-chain, cyclic or branched C₂-C₂₀ hydrocarbon residue which may be interrupted by —O—, —NH—, —NR¹—, —C(O)—, —C(S) and substituted with —OH,
E represents a polyalkylene oxide unit of the structure

—[CH₂CH₂O]_{q}—[CH₂CH(CH₃)O]ᵣ— with,
q 1 to 200,
r 0 to 200 and
A represents —CH₂C(O)O—, —CH₂CH₂C(O)O— or —CH₂CH₂CH₂C(O)O—,
N¹ represents a quaternary ammonium structure

—(R⁴)N⁺(R⁵)—
R⁴ represents a monovalent or divalent straight-chain, cyclic or branched C₁-C₂₀ hydrocarbon residue which may be interrupted by —O—, —NH, —C(O)—, —C(S)— and substituted with —OH,
R⁵ represents R⁴ or a single bond to R⁴ or F,
F represents a divalent or tetravalent straight-chain, cyclic or branched C₂-C₃₀ hydrocarbon residue which may be interrupted by —O—, —NH—, —N—, —C(O)—, —C(S)—, a siloxane chain S, the above-stated relationships applying for S, and substituted with —OH.

The possibility of a tetravalent substructure for F means that F may form a branched or ring system with the adjacent N¹, such that F then participates, in each case with two bonds, in the quaternization of two adjacent N¹. For further illustrative details, reference is made to published patent application WO 02/10257, in particular Example 1 therein.

In a further embodiment of polyammonium/polysiloxane compounds, the possibility of a divalent substructure for R⁴ means that these cases involve a structure which forms cyclic systems in which R⁵ is in this case a single bond to R⁴. Examples are morpholinyl and piperidinyl structures. More preferred embodiments of this so-called first embodiment of the invention and methods of producing the stated polysiloxane compounds of the formula (I) or (I′) are described below.

R⁴ is preferably —CH₃, —CH₂CH₃, —(CH₂)₂CH₃, —(CH₂)₃CH₃, —(CH₂)₅CH₃, —CH₂CH₂OH, —CH₂CH₂NHCO—R¹⁴ or —CH₂CH₂CH₂NHCO—R¹⁴, in which R¹⁴ is a straight-chain, cyclic or branched C₁-C₁₈ hydrocarbon residue which may be interrupted by —O—, —NH—, —C(O)—, —C(S)— and substituted with —OH.

As mentioned above, R⁴ and R⁵ may also together form a cyclic structure of the formulae

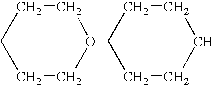

With regard to the preferred meanings of R¹ in the so-called first embodiment of the polysiloxane compounds, reference may be made to the above explanations.

In the so-called first embodiment of the polysiloxane compounds, R⁴ is preferably a monovalent or divalent straight-chain, cyclic or branched C₁-C₁₆, more preferably C₃-C₁₆ hydrocarbon residue which may be interrupted by —O—, —NH—, —C(O)—, —C(S)— and substituted with —OH, more preferably a C₃-C₁₆ hydrocarbon residue which may be interrupted by —O—, —NH—, —NR¹—, —C(O)—, —C(S)— and substituted with —OH, in which R¹ has the above-stated meaning.

In the so-called first embodiment of the polysiloxane compounds, F is preferably a divalent or tetravalent straight-chain, cyclic or branched C₂-C₂₀ hydrocarbon residue which may be interrupted by —O—, —NH—, —N—, —C(O)—, —C(S), a siloxane chain S, the above-stated relationships applying for S, and substituted with —OH.

In the so-called first embodiment of the polysiloxane compounds, K is preferably —CH₂CH₂CH₂—, —(CH₂)₄—, —(CH₂)₆—, —CH₂CH₂CH₂OCH₂CH(OH)CH₂—, and —CH═CHCH₂—.

In the so-called first embodiment of the polysiloxane compounds, R¹⁴ preferably represents unsubstituted C₅-C₁₇ hydrocarbon residues which are derived from the corresponding fatty acids or alternatively hydroxylated C₃-C₁₇ residues, which may be attributed to hydroxylated carboxylic acids, preferably saccharide carboxylic acids.

In the so-called first embodiment of the polyammonium/polysiloxane compounds, which in the present invention are used as active ingredients a) of the active ingredient complex according to the invention, R¹⁴ furthermore preferably represents hydroxylated residues from the group consisting of

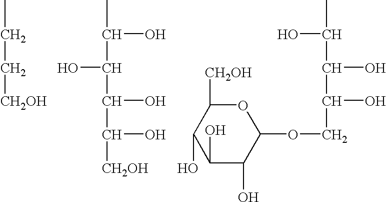

In the so-called first embodiment of the polysiloxane compounds, m is 2 to 100, preferably 2 to 50.

In the so-called first embodiment of the polysiloxane compounds n is 0 to 1000, preferably 0 to 100, more preferably 0 to 80 and particularly preferably 10 to 80.

In the so-called first embodiment of the invention q is 1 to 200, preferably 1 to 50, more preferably 2 to 20 and particularly preferably 2 to 10.

In the so-called first embodiment of the invention r is 0 to 200, preferably 0 to 100, more preferably 0 to 50 and still more preferably 0 to 20.

With regard to the production of the polysiloxane/polyammonium compounds according to the invention both of this first embodiment and also of all further preferred embodiments of the polysiloxane/polyammonium compounds a) according to the invention of the active ingredient complex according to the invention, very explicit reference is made to published patent application WO 02/10257.

One particular embodiment of the invention (hereinafter designated the so-called second embodiment of the polysiloxane compounds) is represented by the polysiloxane compounds of the general formula (II),

R²-E-A-N²—K—S—K—N²-A-E-R² (II)

in which
S, K, -A-E-, -E-A- and R² have the above-stated meanings, and N² is an organic residue which contains at least one quaternary ammonium group of the general formula —(R⁸)N⁺(R⁹)—,
in which
R⁸ represents a monovalent or divalent straight-chain, cyclic or branched C₁-C₂₀ hydrocarbon residue which may be interrupted by —O—, —NH—, —C(O)—, —C(S)— and substituted with —OH,
R⁹ represents a monovalent straight-chain, cyclic or branched C₁-C₂₀ hydrocarbon residue which may be interrupted by —O—, —NH—, —C(O)—, —C(S)— and substituted with —OH, or a single bond to a divalent residue R⁸ or to a trivalent residue K, and residues R⁸ and R⁹ within the polysiloxane compound of the general formula (II) may be identical or different from one another.

The polysiloxane compounds of the second embodiment preferably comprise α,ω-alkylene oxide- and polyquaternary-modified polysiloxanes of the general formula (II′),

R¹⁶-E-A-N²—K—S—K—N²-A-E-R¹⁶ (II′)

in which the designations denote,
S —Si(R¹)₂—O[—Si(R¹)₂—O]ₙ—Si(R¹)—
with R¹ C₁-C₂₂ alkyl, C₁-C₂₂ fluoroalkyl or aryl,
n means 0 to 1000,
K represents a divalent or trivalent straight-chain, cyclic or branched C₂-C₂₀ hydrocarbon residue which may be interrupted by —O—, —N—, —NH—, —NR¹—, —C(O)—, —C(S)— and substituted with —OH,
N² represents a quaternary ammonium structure

—(R⁸)N⁺(R⁹)—
R⁸ represents a monovalent or divalent straight-chain, cyclic or branched C₁-C₂₀ hydrocarbon residue which may be interrupted by —O—, —NH—, C(O)—, —C(S)— and substituted with —OH,
R⁹ represents R⁸ or a single bond to K or R⁸,
A represents —CH₂C(O)O—, —CH₂CH₂C(O)O— or —CH₂CH₂CH₂C(O)O—
E represents a polyalkylene oxide unit of the structure

—[CH₂CH₂O]_{q}—[CH₂CH(CH₃)O]ᵣ—
q 1 to 200
r 0 to 200 and
R¹⁶ represents H, straight-chain, cyclic or branched C₁-C₂₀ hydrocarbon residue which may be interrupted by —O—, or —C(O)— and substituted with —OH and be acetylenic, olefinic or aromatic.

The possibility of a trivalent substructure for K here means that K may be branched and then participates with two bonds in the quaternization of N². The possibility of a divalent substructure for R⁸ means that these cases involve a structure which forms cyclic systems, R⁹ then being a single bond to R².

R⁸ is preferably —CH₃, —CH₂CH₃, —(CH₂)₂CH₃, —(CH₂)₃CH₃, —(CH₂)₅CH₃, —CH₂CH₂OH, —CH₂CH₂NHCO—R¹⁷ or —CH₂CH₂CH₂NHCO—R¹⁷,

in which R¹⁷ is a straight-chain, cyclic or branched C₁-C₁₈ hydrocarbon residue which may be interrupted by —O—, —NH—, —C(O)—, —C(S)— and substituted with —OH.

As mentioned above, R⁸ and R⁹ may also together form a cyclic structure of the formulae

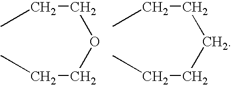

With regard to the preferred meanings of R¹ in the so-called second embodiment of the polysiloxane compounds, reference may be made to the above explanations.

In the so-called second embodiment of the polysiloxane compounds, K is preferably a divalent or trivalent straight-chain, cyclic or branched C₃-C₁₆ hydrocarbon residue which may be interrupted by —O—, —NH—, —NR₁, —N—, —C(O)—, —C(S)— and substituted with —OH, in which R¹ is as defined above.

Preferred residues K are for example those of the following structures: —CH₂CH₂CH₂—, —CH₂CH₂CH₂OCH₂CHOHCH₂— or

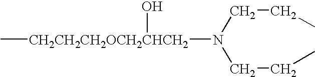

R⁸ is preferably a monovalent or divalent straight-chain, cyclic or branched C₁-C₁₆ hydrocarbon residue which may be interrupted by —O—, —NH—, —C(O), —C(S)— and substituted with —OH.

R¹⁶ is preferably a straight-chain, cyclic or branched C₁-C₁₈ hydrocarbon residue which may be interrupted by —O— or —C(O)— and substituted with —OH and be acetylenic or olefinic.

R¹⁶ is furthermore preferably C₅-C₁₇ alkyl, —CH₂CH═CH₂, —CH₂CH(OH)CH₂OCH₂CH═CH₂, —CH₂CCH, —C(O)CH₃, —C(O)CH₂CH₃.

R¹⁷ preferably represents unsubstituted C₅-C₁₇ hydrocarbon residues, which are derived from the corresponding fatty acids or alternatively hydroxylated C₃-C₁₇ residues which may be attributed to hydroxylated carboxylic acids, preferably to saccharide carboxylic acids.

R¹⁷ is particularly preferably selected from the group

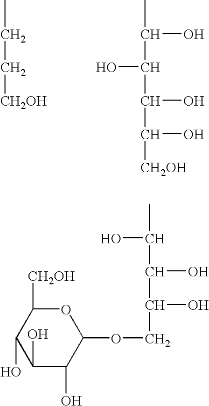

In the so-called second embodiment of the polysiloxane compounds, n is preferably 0 to 200, more preferably 0 to 80, particularly preferably 10 to 80.

In the so-called second embodiment of the polysiloxane compounds, q is preferably 1 to 50, more preferably 2 to 20 and particularly preferably 2 to 10.

In the so-called second embodiment of the polysiloxane compounds, r is preferably 0 to 100 and more preferably 0 to 50.

In the so-called second embodiment of the invention, r is preferably 0 to 20 and more preferably 0 to 10.

With regard to the production of the polysiloxane compounds according to the invention of the so-called second embodiment, reference is made to the explanations relating to the first preferred embodiment.

One particular embodiment of polyammonium/polysiloxane compounds a) as an essential constituent of the active ingredient complex according to the invention (hereinafter designated the so-called third embodiment of the polysiloxanes) is represented by the polysiloxane compounds of the general formula (III):

—[K—S—K—N³]ₘ— (III)

in which S, K and m are defined as above,
N³ is an organic residue which contains at least one quaternary ammonium group of the general formula

—(R¹⁰)—N⁺(R¹¹)—

in which R¹⁰ represents a monovalent straight-chain, cyclic or branched C₁-C₃₀ hydrocarbon residue which may be interrupted by —O—, —NH—, —C(O)—, —C(S)— and substituted with —OH or a single bond to K,
R¹¹ denotes -A-E-R², in which -A-E-R² has the above-stated meaning.

The polysiloxane compounds of the third embodiment preferably comprise alkylene oxide-modified polyquaternary polysiloxanes of the general formula (III′),

—[K—S—K—N³]ₘ— (III′),

in which m is 2 to 500,
S means —Si(R¹)₂—O[—Si(R¹)₂—O]ₙ—Si(R¹)₂—
with R¹ C₁-C₂₂ alkyl, C₁-C₂₂ fluoroalkyl or aryl,
n=0 to 1000,
N³ represents a quaternary ammonium structure

—(R¹⁰)N⁺(R¹¹)—
in which R¹⁰ represents a monovalent or divalent straight-chain, cyclic or branched C₁-C₃₀ hydrocarbon residue which may be interrupted by —O—, —NH—, —C(O)—, —C(S)— and substituted with —OH, or a single bond to K,
R³ is -A-E-, with
A denoting —CH₂C(O)O—, —CH₂CH₂C(O)O— or —CH₂CH₂CH₂C(O)O— and
E denoting a polyalkylene oxide unit of the structure

—[CH₂CH₂O]_{q}—[CH₂CH(CH₃)O]ᵣ—R¹⁸
q from 1 to 200,
r from 0 to 200,
R¹⁸ denoting H, straight-chain, cyclic or branched C₁-C₂₀ hydrocarbon residue which may be interrupted by —O—, or —C(O)— and substituted with —OH and be acetylenic, olefinic or aromatic, and
K is a divalent or trivalent straight-chain, cyclic or branched C₂-C₄₀ hydrocarbon residue which may be interrupted by —O—, —NH—, —NR¹—, —N—, —C(O)—, —C(S)— and substituted with —OH or contains a quaternary ammonium structure N⁵, with N⁵ meaning —(R¹⁹)N⁺(R²⁰)—
R¹⁹ represents a monovalent or divalent straight-chain, cyclic or branched C₁-C₂₀ hydrocarbon residue which may be interrupted by —O—, —NH—, —C(O)—, —C(S)— and substituted with —OH or a single bond to R¹⁰, and R²⁰ is -A-E-, which is defined as above.

With regard to the production of the preferred embodiments of the so-called third embodiment of the polysiloxane compounds, explicit reference is made as previously to published patent application WO 02/10257.

R¹⁰ and R¹⁹ are mutually independently preferably —CH₃, —CH₂CH₃, —(CH₂)₂CH₃, —(CH₂)₃CH₃, —(CH₂)₅CH₃, —CH₂CH₂OH, —CH₂CH₂NHCOR²¹ or —CH₂CH₂CH₂NHCOR²¹, in which R²¹ is a straight-chain, cyclic or branched C₁-C₁₈ hydrocarbon residue which may be interrupted by —O—, —NH—, —C(O)—, —C(S)— and substituted with —OH.

In one embodiment of the so-called third embodiment of the polysiloxane compounds, a divalent substructure for R¹⁰ comprises a structure which forms a cyclic system, R¹⁰ then having a single bond to K, preferably to a tertiary amino structure or alternatively to the quaternary structure N⁵ via R¹⁹.

With regard to the preferred meanings of R¹ in the so-called third embodiment of the polysiloxanes, reference may be made to the above explanations.

R¹⁰ is preferably a monovalent or divalent straight-chain, cyclic or branched C₁-C₂₅ hydrocarbon residue which may be interrupted by —O—, —NH—, —C(O)—, —C(S)— and substituted with —OH.

R¹⁹ is preferably a monovalent or divalent straight-chain, cyclic or branched C₁-C₂₅ hydrocarbon residue which may be interrupted by —O—, —NH—, —C(O)—, —C(S)— and substituted with —OH.

In the so-called third embodiment of the polysiloxane compounds, K is furthermore preferably a divalent or trivalent straight-chain, cyclic or branched C₃-C₃₀ hydrocarbon residue which may be interrupted by —O—, —NH—, —NR¹—, —N—, —C(O)—, —C(S)— and substituted with —OH; K is still more preferably —CH₂CH₂CH₂OCH₂CHOHCH₂—,

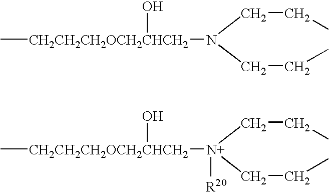

in which R²⁰ is defined as above.

In the so-called third embodiment of the polysiloxanes, R² or R¹⁸ is preferably a straight-chain, cyclic or branched C₁-C₁₈ hydrocarbon residue which may be interrupted by —O— or —C(O)— and —OH substituted and be acetylenic or olefinic. R² or R¹⁸ is more preferably C₁-C₆ alkyl, —CH₂CH═CH₂, —CH₂CH(OH)CH₂OCH₂CH═CH₂, —CH₂CCH, —C(O)CH₃ or —C(O)CH₂CH₃.

R²¹ is preferably an unsubstituted C₅-C₁₇ hydrocarbon residue which is derived from the corresponding fatty acids or alternatively comprises hydroxylated C₃-C₁₇ residues and originates from the group of hydroxylated carboxylic acids, preferably saccharide carboxylic acids.

R²¹ is accordingly for example:

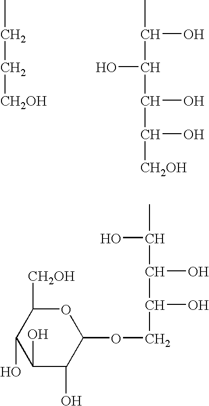

In the so-called third embodiment of polysiloxanes, m is preferably 2 to 100, and particularly preferably 2 to 50, n is 0 to 100, preferably 0 to 80, and particularly preferably 10 to 80, q is 1 to 50, preferably 2 to 50, particularly preferably 2 to 20, and still more preferably q is 2 to 10, r is 0 to 100, preferably 0 to 50, particularly preferably 0 to 20, and still more preferably r is 0 to 10.

With regard to the production of polysiloxane-compounds to be used according to the invention of the so-called third embodiment reference may again conveniently made to published patent application WO 02/10257.

One particular embodiment of the polysiloxanes (hereinafter designated so-called fourth embodiment of the polysiloxanes to be used according to the invention) is represented by the polysiloxane compounds of the general formula (IV):

—[N⁴—K—S—K—N⁴-A-E-A′]ₘ and —[N⁴—K—S—K—N⁴-A′-E-A]ₘ— (IV)

in which m, K, S, -A-E-A′- and -A′-E-A- are defined as above, and
N⁴ is an organic residue which contains at least one quaternary ammonium group of the general formula —(R¹²)N⁺(R¹³)—, in which R¹² is a monovalent or divalent straight-chain, cyclic or branched C₁-C₂₀ hydrocarbon residue which may be interrupted by —O—, —NH—, —C(O)—, —C(S)— and substituted with —OH,
R¹³ may have the meanings of R¹² or represents a single bond to K or R¹², and the residues R¹² and R¹³ may be identical or different from one another.

The polysiloxane compounds of the fourth embodiment preferably comprise alkylene oxide-modified polyquaternary polysiloxanes of the general formula (IV′),

—[N⁴—K—S—K—N⁴-A-E-A]ₘ— (IV′)

in which m=2 to 500,
S —Si(R¹)₂—O[—Si(R¹)₂—O]—Si(R¹)₂—, in which
R¹ denotes C₁-C₂₂ alkyl, C₁-C₂₂ fluoroalkyl or aryl,
n represents 0 to 1000,
K represents a divalent or trivalent straight-chain, cyclic or branched C₂-C₂₀ hydrocarbon residue which may be interrupted by —O—, —NH—, —NR¹, —N—, —C(O)—, —C(S)— and substituted with —OH,
N is a quaternary ammonium structure —(R¹²)N⁺(R¹³)—, in which R¹² represents a monovalent or divalent straight-chain, cyclic or branched C₁-C₂₀hydrocarbon residue, which may be interrupted by —O—, —NH—, —C(O)—, —C(S)— and substituted with —OH,
R¹³ denotes R¹² or a single bond to K or R¹²,
A is —CH₂C(O)O—, —CH₂CH₂C(O)O— or —CH₂CH₂CH₂C(O)O—
E is a polyalkylene oxide unit of the structure

—[CH₂CH₂O]_{q}—[CH₂CH(CH₃)O]ᵣ—

with
q=1 to 200 and
r=0 to 200.

With regard to production methods, reference is made to the previous citations.

More preferred embodiments of this so-called fourth embodiment of polysiloxanes of the formula (IV) or (IV′) are described below.

The possibility of a trivalent substructure for K means that K may be branched and then may participate with two bonds in the quaternization of N⁴.

The possibility of a divalent substructure for R¹² means that these cases involve a structure which forms cyclic systems, R¹³ then being a single bond to R¹².

R¹² is preferably —CH₃, —CH₂CH₃, —(CH₂)₂CH₃, —(CH₂)₃CH₃, —(CH₂)₅CH₃, —CH₂CH₂OH, —CH₂CH₂NHCOR²² or —CH₂CH₂CH₂NHCOR²²,

in which R²² is a straight-chain, cyclic or branched C₁-C₁₈ hydrocarbon residue which may be interrupted by —O—, —NH—, —C(O)—, —C(S)— and substituted with —OH.

As mentioned above, R¹² and R¹³ may also together form a cyclic structure of the formulae

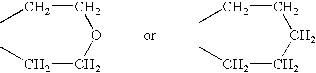

With regard to the preferred meanings of R¹ in the so-called fourth embodiment of the polysiloxanes, reference may be made to the above explanations.

R¹² is preferably a monovalent or divalent straight-chain, cyclic or branched C₁-C₁₆ hydrocarbon residue which may be interrupted by —O—, —NH—, —C(O)—, —C(S)— and substituted with —OH.

In the so-called fourth embodiment, K is preferably a divalent or trivalent straight-chain, cyclic or branched C₃-C₁₆ hydrocarbon residue which may be interrupted by —O—, —NH—, —NR¹—, —N—, —C(O)—, —C(S)— and substituted with —OH; K is particularly preferably —CH₂CH₂CH₂—, —CH₂CH₂CH₂OCH₂CHOHCH₂— or

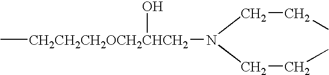

R²² is preferably an unsubstituted C₅-C₁₇ hydrocarbon residue which is derived from the corresponding fatty acids or alternatively comprises hydroxylated C₃-C₁₇ residues which may be attributed to hydroxylated carboxylic acids, preferably saccharide carboxylic acids.

R²² is more preferably:

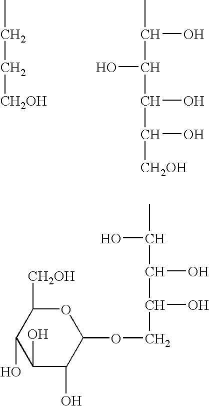

m is preferably 2 to 100, and particularly preferably 2 to 50. n is 0 to 100, preferably 0 to 80, and particularly preferably 10 to 80. q is 1 to 50, preferably 2 to 50, and particularly preferably 2 to 20, q is still more preferably 2 to 10. r is 0 to 100, preferably 0 to 50, and particularly preferably 0 to 20, r is still more preferably 0 to 10.

The phrase “C₁-C₂₂ alkyl or C₁-C₃₀ hydrocarbon residue”, as used above, means for the purposes of the present invention aliphatic hydrocarbon compounds with 1 to 22 carbon atoms or 1 to 30 carbon atoms, which may be straight-chain or branched. Methyl, ethyl, propyl, n-butyl, pentyl, hexyl, heptyl, nonyl, decyl, undecyl, iso-propyl, neopentyl and 1,2,3-trimethylhexyl may be listed by way of example.

The term “C₁-C₂₂ fluoroalkyl”, as used above, means for the purposes of the present invention aliphatic hydrocarbon compounds with 1 to 22 carbon atoms which may be straight-chain or branched and are substituted with at least one fluorine atom. Monofluoromethyl, monofluoroethyl, 1,1,1-trifluoroethyl, perfluoroethyl, 1,1,1-trifluoropropyl, 1,2,2-trifluorobutyl may be listed by way of example.

The term “aryl”, as used above, means for the purposes of the present invention phenyl which is unsubstituted or mono- or polysubstituted with OH, F, Cl, CF₃, C₁-C₆ alkyl, C₁-C₆ alkoxy, C₃-C₇ cycloalkyl, C₂-C₆ alkenyl or phenyl. The expression may optionally also mean naphthyl.

One particular embodiment of the polysiloxanes according to the invention as constituent a) of the active ingredient complex according to the invention (hereinafter designated so-called fifth embodiment of the polysiloxanes) is represented by the polysiloxanes of the general formula (V):

[—N⁵—F¹—N⁵—Y—]ₘ

in which
Y is a group of the formula —K—S—K— and -A-E-A′- or -A′-E-A-,
in which m, K, S, -A-E-A′- and -A′-E-A- are defined as above, the groups K, S, -A-E-A′- and -A′-E-A- within the polysiloxanes of the general formula (V) may be identical or different from one another, and the molar ratio of the group —K—S—K— and the group -A-E-A′- or -A′-E-A- in the polysiloxane compound of the general formula (V) is from 100:1 to 1:100,
N⁵ is an ammonium group of the general formula —(R²³)N⁺(R²⁴)—, in which
R²³ represents hydrogen, a monovalent or divalent straight-chain, cyclic or branched C₁-C₂₀ hydrocarbon residue, which may be interrupted by —O—, —NH—, —C(O)—, —C(S)— and substituted with —OH,
R²⁴ represents hydrogen, a monovalent straight-chain, cyclic or branched C₁-C₂₀ hydrocarbon residue, which may be interrupted by —O—, —NH—, —C(O)—, C(S)— and substituted with —OH, or represents a single bond to a divalent residue R²³, and the residues R²³ and R²⁴ within the group —N⁵—F¹—N⁵— and in the polysiloxane compound may be identical or different from one another,
F¹ represents a divalent straight-chain, cyclic or branched hydrocarbon residue which may be interrupted by —O—, —NH—, —N—, —C(O)— or —C(S)— or by a group -E-, in which E is defined as above, and in which a plurality of N⁵ and F¹ may in each case be identical or different from one another.

The molar ratio of group —K—S—K— and group -A-E-A′- or -A′-E-A- in the polysiloxane compound of the general formula (V) is between 100:1 and 1:100. This molar ratio may, as shown in published patent application WO 02/10257, be controlled by selection of the molar ratio of starting compounds, in particular the ratio of the α,ω-halocarboxylic acid polyalkylene oxide ester compounds and the polysiloxane/bisepoxide compounds preferably used according to the invention. The properties of the products are substantially dependent on the ratio of starting materials used and on the length of the polyalkylene oxide or polysiloxane blocks contained therein.

In a preferred embodiment of the so-called fifth embodiment of polysiloxanes, K is a divalent hydrocarbon residue with at least 4 carbon atoms which comprises a hydroxyl group and which may be interrupted by an oxygen atom.

In a preferred embodiment of the so-called fifth embodiment of the polysiloxanes, F¹ is a divalent straight-chain, cyclic or branched C₂-C₃₀ hydrocarbon residue which may be interrupted by —O—, —NH—, —N—, —C(O)—, —C(S)— or by a group -E-, in which E is defined as above, and in which the carbon atoms arising from the residue E do not count towards the 2 to 30 carbon atoms of the C₂-C₃₀ hydrocarbon residue.

In a further preferred embodiment of the so-called fifth embodiment of the invention

—N⁵—F¹—N⁵—

is a group of the formula:

—N(R²⁵R²⁶)⁺—F²—N(R²⁵R²⁶)⁺—

in which
R²⁵ is a monovalent or divalent straight-chain, cyclic or branched C₁-C₂₀ hydrocarbon residue which may be interrupted by —O—, —NH—, —C(O)—, —C(S)— and substituted with —OH, particularly preferably methyl,
R²⁶ is a monovalent straight-chain, cyclic or branched C₁-C₂₀ hydrocarbon residue which may be interrupted by —O—, —NH—, —C(O)—, —C(S) and substituted with —OH, particularly preferably methyl, or represents a single bond to a divalent residue R²⁵, and the residues R²⁵ and R²⁶ within the group —N⁵—F²—N⁵— and in the polysiloxane compound may be identical or different from one another, and
F² is a divalent straight-chain, cyclic or branched hydrocarbon residue which may be interrupted by —O—, —NH—, —N—, —C(O)—, —C(S)—.

In a still more preferred embodiment, F² is a branched, preferably straight-chain C₁-C₆ alkanediyl group, among which a 1,6-hexanediyl (or hexamethylene) group is preferred.

In a further preferred embodiment of the so-called fifth embodiment of the polysiloxane compounds,

—N⁵—F¹—N⁵—

is a group of the formula:

—N(R²⁷R²⁸)⁺—F³—N(R²⁷R²⁸)⁺—

in which
R²⁷ and R²⁸ are in each case hydrogen, C₁-C₆ alkyl or hydroxy-(C₁-C₆)-alkyl, preferably hydrogen, methyl or —CH₂CH₂OH, and
F³ is a divalent straight-chain, cyclic or branched hydrocarbon residue which is interrupted by a group -E-, in which E is defined as above.

F³ is particularly preferably a group of the formula

-D-E-D-

in which E is defined as above and D is in each case a single bond or a straight-chain or branched C₁-C₆ alkanediyl group, with the proviso that D is not a single bond if it bonds to a terminal oxygen atom of group E.

The group -D-E-D- is preferably represented by a group of the formula

-D-(OCH₂CH₂)ᵥ(OCH₂CH(CH₃))_{w}—O-D-

in which D is a straight-chain or branched C₁-C₆ alkanediyl group and r and q are defined as above. In the group -D-(OCH₂CH₂)_{q}(OCH₂CH(CH₃))ᵣO-D-, the ethylene oxide and propylene oxide units may be arranged as desired, for example as a random copolymer unit or as a block copolymer unit.

v is preferably 1 to 100, more preferably 1 to 70, still more preferably 1 to 40.

w is preferably 0 to 100, more preferably 0 to 70, still more preferably 0 to 40.

In a further preferred embodiment of the so-called fifth embodiment of the invention the group

—N⁵—F¹—N⁵

is represented by a group of the formula:

—N⁺R²⁵R²⁶—F²—N⁺R²⁵R²⁶—

and a group of the formula:

—N⁺R²⁷R²⁸—F³—N⁺R²⁷R²⁸—

in which the substituents in each case have the above meanings.

This means that the polysiloxane compounds of the general formula (V) are made up of two different types of group —N⁵—F¹—N⁵—.

In this embodiment, the molar ratio of the group

—N⁺R²⁵R²⁶—F²—N⁺R²⁵R²⁶—

to the group

—N⁺R²⁷R²⁸—F³—N⁺R²⁷R²⁸—

conveniently amounts to 70:30 to 95:5, preferably 80:20 to 90:10.

The polysiloxane compounds of the general formula (V) may be cyclic or linear. In the case of linear compounds, the terminal groups arise either from the difunctional monomers described below which were used for production or the functionalized derivatives thereof or from monoamines which are added as chain terminators during polymerization. The terminal groups arising from the use of monoamine chain terminators preferably assume the form of ammonium groups, either by quaternization or protonation.

In a further preferred embodiment of the so-called fifth embodiment of polysiloxanes, K denotes one of the groups of the formula:

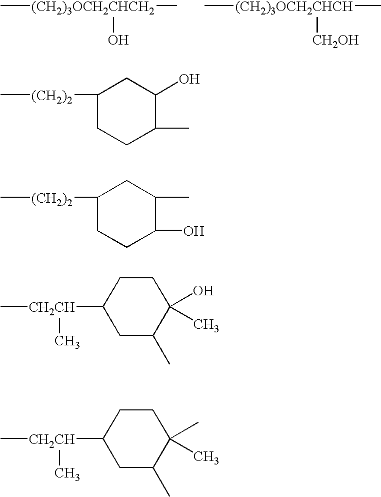

In the so-called fifth embodiment of the polysiloxanes, q is preferably in the range from 1 to 50, in particular 2 to 50, specifically 2 to 20 and very specifically 2 to 10, and r is in the range from 0 to 100, in particular 0 to 50, specifically 0 to 20 and very specifically 0 to 10.

In the so-called fifth embodiment of the invention, the organic or inorganic acid residue for neutralizing the charges arising from the ammonium group(s) is conveniently selected from inorganic residues, such as chloride, bromide, hydrogensulfate, sulfate, or organic residues, such as acetate, propionate, octanoate, decanoate, dodecanoate, tetradecanoate, hexadecanoate, octadecanoate and oleate, with, as initially mentioned, chloride and bromide preferably arising from the reaction of the alkyl halide groups with amine groups.

The polysiloxanes of the fifth embodiment furthermore assume protonated form as amine salts or as amines.

The polysiloxanes of the fifth embodiment of the invention are conveniently produced by one of the methods which are described in published patent application WO 02/10257.

The above-described polyammonium/polysiloxane compounds may for example be obtained from GE Bayer Silicones under the tradename Baysilone®. The products named Baysilone TP 3911, SME 253 and SFE 839 are here preferred. It is very particularly preferred to use Baysilone TP 3911 as an active component of the compositions according to the invention.

The above-described polyammonium/polysiloxane compounds are used in the compositions according to the invention in a quantity of 0.01 to 10 wt. %, preferably 0.01 to 7.5, particularly preferably 0.01 to 5.0 wt. %, very particularly preferably of 0.05 to 2.5 wt. % in each case relative to the total composition.

If a mixture of at least two silicones is used, this mixture is present in the compositions according to the invention in quantities of 0.01 to 10 wt. %, preferably of 0.01 to 8 wt. %, particularly preferably of 0.1 to 7.5 wt. % and in particular of 0.1 to 5 wt. % of silicone mixture relative to the composition.

It is also possible according to the invention for the mixture of the silicones to form their own phase in the compositions according to the invention. In this case it may be appropriate for the composition to be briefly homogenized by shaking immediately before application. In this case, the quantity of silicone mixture may amount to up to 40 wt. %, preferably in quantities of up to 25 wt. % relative to the total composition.

Surface-active substances are constituents which are used in virtually all cosmetic compositions. The surface-active substances substantially comprise two groups, surfactants and emulsifiers. These constituents may be included in the compositions according to the invention in order to establish certain applicational properties of the compositions. The addition of surfactants, for example, may serve to adjust foaming behavior with regard to the amount and spontaneity of foaming. Emulsifiers may serve, for example, to incorporate perfume oils into the compositions according to the invention.

The term surfactants (E) is understood to mean surface-active substances, which form adsorption layers on surfaces and interfaces or may aggregate in volume phases to yield micellar colloids or lyotropic mesophases. A distinction may be drawn between anionic surfactants consisting of a hydrophobic residue and a negatively charged hydrophilic head group, amphoteric surfactants, which bear both a negative and a compensating positive charge, cationic surfactants, which, in addition to a hydrophobic residue, comprise a positively charged hydrophilic group, and nonionic surfactants, which do not comprise any charges but rather comprise strong dipole moments and are strongly hydrated in aqueous solution. Further definitions and properties of surfactants may be found in “H.-D. Dörfler, Grenzflächen- und Kolloidchemie [interfacial and colloid chemistry], VCH Verlagsgesellschaft mbH, Weinheim, 1994”. All the surfactants stated below are known compounds. With regard to the structure and production of these substances, reference is made to relevant review articles.

Anionic surfactants (E1) which are suitable in preparations according to the invention are any anionic surface-active substances suitable for use on the human body. These are characterized by an anionic water-solubilizing group such as for example a carboxylate, sulfate, sulfonate or phosphate group and a lipophilic alkyl group having some 8 to 30 C atoms. The molecule may additionally contain glycol or polyglycol ether groups, ester, ether and amide groups and hydroxyl groups. Typical examples of anionic surfactants are: alkylbenzene sulfonates, alkane sulfonates, olefin sulfonates, alkyl ether sulfonates, glycerol ether sulfonates, α-methyl ester sulfonates, sulfofatty acids, alkyl sulfates, fatty alcohol ether sulfates, glycerol ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and the salts thereof, fatty acid isethionates, fatty acid sarcosinates, fatty acid taurides, acyl lactylates, acyl tartrates, acyl glutamates, acyl aspartates, alkyl oligoglucoside sulfates, protein fatty acid condensation products (in particular wheat-based plant products) and alkyl (ether) phosphates. If the anionic surfactants contain polyglycol ether chains, these may exhibit a conventional, but preferably a narrow homolog distribution. Examples of suitable anionic surfactants are, in each case in the form of sodium, potassium and ammonium and the mono-, di- and trialkanolammonium salts having 2 to 4 C atoms in the alkanol group, linear and branched fatty acids having 8 to 30 C-atoms (soaps), linear alkane sulfonates having 8 to 24 C atoms, linear alpha-olefin sulfonates with 8 to 24 C atoms, alpha-sulfofatty acid methyl esters of fatty acids having 8 to 30 C atoms, alkyl sulfates and alkyl polyglycol ether sulfates of the formula R—O(CH₂—CH₂O)ₓ—OSO₃H, in which R is a preferably linear alkyl group having 8 to 30 C atoms and x=0 or 1 to 12, mixtures of surface-active hydroxysulfonates according to DE-A-37 25 030, sulfated hydroxyalkyl polyethylene and/or hydroxyalkylene propylene glycol ethers according to DE-A-37 23 354, sulfonates of unsaturated fatty acids having 8 to 24 C atoms and 1 to 6 double bonds according to DE-A-39 26 344, sulfated fatty acid alkylene glycol esters of the formula (E1-II)

R⁷CO(AlkO)ₙSO₃M (E1-II)

in which R⁷CO— denotes a linear or branched, aliphatic, saturated and/or unsaturated acyl residue with 6 to 22 C atoms, Alk denotes CH₂CH₂, CHCH₃CH₂ and/or CH₂CHCH₃, n denotes numbers from 0.5 to 5 and M denotes a cation, as they are described in DE-OS 197 36 906.5.

Preferred anionic surfactants are alkyl sulfates, alkyl polyglycol ether sulfates and ether carboxylic acids having 10 to 18 C atoms in the alkyl group and up to 12 glycol ether groups per molecule, sulfosuccinic acid mono- and dialkyl esters having 8 to 18 C atoms in the alkyl group and sulfosuccinic acid monoalkyl polyoxyethyl esters having 8 to 18 C atoms in the alkyl group and 1 to 6 oxyethyl groups, monoglyceryl disulfates, alkyl and alkenyl ether phosphates and protein/fatty acid condensation products.

In the compositions according to the invention, it is particularly advantageous for the anionic surfactants to be mild anionic surfactants. The action of the composition according to the invention is increased substantially in particular by the mild anionic surfactants described below.

The term “mild surfactants” is understood by the person skilled in the art to mean surfactants which have proven to be mild surfactants using numerous test methods, such as the HET-CAM test, the neutral red test, the BUS model (bovine udder skin model), the human skin model, the zein test, the Draize test, the arm flex wash test or the Duhring Chamber Test etc. Common to all test models is the fact that measurement is in principle performed against a standard to which the measurement results are related. In each of the test models there is a threshold value below which surfactants are spoken of as “mild surfactants”. This threshold value amounts for example in the HET-CAM test to 1.5. This means all surfactants are designated “mild” which achieve a relative irritancy score of 1.5 or less in the HET-CAM test. The person skilled in the art knows that a surfactant achieves a different score in each test model. This means that for example a cocamidopropyl betaine may even be classed as “irritant” in the HET-CAM test, while in the other test models it is more likely to be grouped with the mild surfactants. One recognized common classification defines anionic surfactants as mild if they achieve a relative irritancy score of less than 1.5 in the HET-CAM test. According to the invention, however, anionic surfactants which are preferably used and understood as “mild anionic surfactants” are those which are classified as “mild” in all the currently familiar test models.

According to these test methods the following anionic surfactants have proven to be mild to particularly mild and are particularly preferred according to the invention:

acyl lactylates, hydroxy mixed ether sulfates, ether carboxylic acids of the formula R—O—(CH₂—CH₂O)ₓ—CH₂—COOH, in which R is a linear alkyl group having 8 to 30 C atoms and x=0 or 1 to 16 and the salts thereof, acyl sarcosides having 8 to 24 C atoms in the acyl group, acyl taurides having 8 to 24 C atoms in the acyl group, acyl isethionates having 8 to 24 C atoms in the acyl group, are long known, skin-friendly surface-active substances, which are obtainable by esterifying fatty acids with the sodium salt of 2-hydroxyethanesulfonic acid (isethionic acid). If fatty acids having 8 to 24 C atoms, i.e. for example lauric, myristic, palmitic or stearic acid, or indeed technical fatty acid fractions, for example the C₁₂-C₁₈ fatty acid fraction obtainable from coconut fatty acid are used for this esterification, C₁₂-C₁₈ acyl isethionate is obtained, which is preferably suitable according to the invention. It is known, as with fatty acid-based soaps, to make sodium salts of C₁₂-C₁₈ acyl isethionates into a form suitable for transportation and application by kneading, milling, extrusion, cutting and tabletting. In this way, needles, granules, noodles, bars and handy toilet soap tablets may be produced. Sulfosuccinic acid mono- and dialkyl esters having 8 to 24 C atoms in the alkyl group and sulfosuccinic acid monoalkyl polyoxyethyl esters having 8 to 24 C atoms in the alkyl group and 1 to 6 oxyethyl groups. Sulfosuccinic acid monoalkyl-(C₈-C₂₄)-ester disodium salts are produced using a known method, for example by reacting maleic anhydride with a fatty alcohol having 8-24 C atoms to yield the maleic acid monoester of the fatty alcohol and by sulfiting the latter with sodium sulfite to yield the sulfosuccinic acid ester. Particularly suitable sulfosuccinic acid esters are derived from fatty alcohol fractions having 12-18 C atoms, as are obtainable for example by hydrogenation from coconut fatty acid or coconut fatty acid methyl ester. Alkyl polyglycol ether sulfates of the formula R—O(CH₂—CH₂O)ₓ—OSO₃H, in which R is a preferably linear alkyl group having 8 to 30 C atoms and x=0 or 1 to 12, esters of tartaric acid and citric acid with alcohols, which represent addition products of for instance 2-15 molecules of ethylene oxide and/or propylene oxide onto fatty alcohols having 8 to 22 C atoms, alkyl and/or alkenyl ether phosphates of the formula (E1-I),

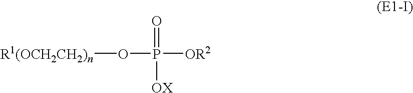

in which R¹ preferably denotes an aliphatic hydrocarbon residue with 8 to 30 carbon atoms, R² denotes hydrogen, a residue (CH₂CH₂O)ₙR² or X, n denotes numbers from 1 to 10 and X denotes hydrogen, an alkali or alkaline earth metal or NR³R⁴R⁵R⁶, with R³ to R⁶ mutually independently denoting hydrogen or a C₁ to C₄ hydrocarbon residue), glycerol ether sulfates such as monoglyceride sulfates and monoglyceride ether sulfates of the formula (E1-III),

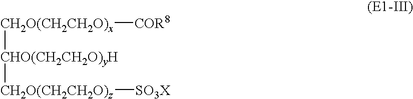

in which R⁸CO denotes a linear or branched acyl residue having 6 to 22 carbon atoms, x, y and z in total denote 0 or denote numbers from 1 to 30, preferably 2 to 10, and X denotes an alkali or alkaline earth metal. Typical examples of monoglyceride (ether) sulfates suitable for the purposes of the invention are the reaction products of lauric acid monoglyceride, coconut fatty acid monoglyceride, palmitic acid monoglyceride, stearic acid monoglyceride, oleic acid monoglyceride and tallow fatty acid monoglyceride and the ethylene oxide addition products thereof with sulfur trioxide or chlorosulfonic acid in the form of the sodium salts thereof. Preferably, monoglyceride sulfates of the formula (E1-III) are used, in which R⁸CO denotes a linear acyl residue having 8 to 18 carbon atoms, amide ether carboxylic acids,
condensation products from a water-soluble salt of a water-soluble protein hydrolyzate/fatty acid condensation product. These are produced by condensing C₈-C₃₀ fatty acids, preferably fatty acids having 12-18 C atoms, with amino acids, mono-, di- and water-soluble oligopeptides and mixtures of such products, as arise on protein hydrolysis. These protein hydrolyzate/fatty acid condensation products are neutralized with a base and are then preferably present as an alkali, ammonium, mono-, di- or trialkanolammonium salt. Such products have long been commercially available under the trademark Lamepon®, Maypon®, Gluadin®, Hostapon® KCG or Amisoft®, acyl glutamates and acyl aspartates.

If the mild anionic surfactants contain polyglycol ether chains, it is very particularly preferable for them to exhibit a narrow homolog distribution. Fatty alcohol ether sulfates with a narrow homolog distribution are also known as “narrow range fatty alcohol ether sulfates”. Furthermore, it is preferred, in the case of mild anionic surfactants with polyglycol ether units, for the number of glycol ether groups to amount to 1 to 20, preferably 2 to 15, particularly preferably 2 to 12. Particularly mild anionic surfactants with polyglycol ether groups without a narrow homolog distribution may for example also be obtained if, on the one hand, the number of polyglycol ether groups amounts to 4 to 12 and Zn or Mg ions are selected as the counterion. An example thereof is the commercial product Texapon® ASV.

It goes without saying that all the mild anionic surfactants mentioned above and below may also be used in the form of the salts thereof. Examples of suitable mild anionic surfactants are in each case present in the form of lithium, magnesium, zinc, sodium, potassium and ammonium and mono-, di- and trialkanolammonium salts having 1 to 4 C atoms in the alkanol group. In addition to the ammonium ion as such, the preferred ammonium ions are monomethylammonium, dimethylammonium, trimethylammonium, monoethylammonium, diethylammonium, triethylammonium, monopropylammonium, dipropylammonium, tripropylammonium, monoisopropylammonium, diisopropylammonium, triisopropylammonium, monobutylammonium, dibutylammonium, tributylammonium, monoisobutylammonium, diisobutylammonium, triisobutylammonium, mono-t-butylammonium, di-t-butylammonium, tri-t-butylammonium ions and mixed ammonium ions such as for example methylethylammonium, dimethylethylammonium, methyldiethylammonium, methylpropylammonium, methylethylpropylammonium, ethyldiisopropylammonium, ethyldibutylammonium, ethyldiisobutylammonium ions etc. It goes without saying that the teaching according to the invention also covers the further, not explicitly stated ammonium ions of these alkanolammonium salts.

Further mild anionic surfactants, which are used very particularly preferably in the composition according to the invention, are alkyl and/or alkenyl oligoglycoside carboxylates, sulfates, phosphates and/or isethionates, which are derived from alkyl and/or alkenyl oligoglycosides of the general formula (I),

R—O-(G)ₚ (I)

with the meaning
R C₆₋₂₂ alkyl or C₆₋₂₂ alkenyl,
G glycoside unit which is derived from a sugar having 5 or 6 carbon atoms,
p number from 1 to 10.

Preferably, the mild anionic surfactant is selected from anionic alkyl polyglycosides, ether carboxylic acids, acyl isethionates, protein/fatty acid condensates, taurates, sulfosuccinates, fatty acid amide ether sulfates, NRE fatty alcohol ether sulfates (narrow range fatty alcohol ether sulfates), acyl glutamates and acyl asparaginates and mixtures thereof.

According to the invention, the anionic alkyl polyglucosides, such as alkyl oligoglycoside carboxylates, sulfates, phosphates and/or isethionates, ether carboxylic acids, acyl isethionates and taurates and mixtures thereof are particularly preferably used.

Very particular preference is given to the use of anionic alkyl polyglucosides and ether carboxylic acids and mixtures thereof.

Most particular preference is given to the use of carboxylated alkyl polyglucosides and ether carboxylic acids and mixtures thereof.

If mixtures of at least two different mild anionic surfactants are used as the mild anionic surfactant, the mixing ratio of these surfactants with one another amounts to at least 10:1 to 1:10. Preference is given to a mixing ratio of 5:1 to 1:5, particularly preferably of 2.5:1 to 1:2.5 and most preferably of for instance 1.5:1 to 1:1.5.

It has been found according to the invention that the use of mild anionic surfactants and in particular of alkyl and/or alkenyl oligoglycoside carboxylates, sulfates, phosphates and/or isoethionates in agents for treating keratin fibers, in particular in agents for cleansing and conditioning hair, leads to a reduction in skin irritation. It has additionally been found that, when applying such agents, the color stability of the dyeing result is improved by additionally using mild anionic surfactants, in particular alkyl and/or alkenyl oligoglycoside carboxylates, sulfates, phosphates and/or isoethionates. In particular, this effect is achieved in the event of application to stressed hair. The washing fastness of dyed stressed hair is also improved.

It has additionally been found according to the invention that, when using mild anionic surfactants, in particular alkyl and/or alkenyl oligoglycoside carboxylates, sulfates, phosphates and/or isoethionates in cleansing and care agents, foaming is improved considerably upon application of these agents. The foam is distinguished in particular by a fine-pored, dense, creamy appearance. The foam is described as pleasantly soft and pliable and easy to spread. At the same time, the foam is firm and easily held. It displays a degree of stability and does not run spontaneously until a few minutes have elapsed. This promotes the above-described easy spreadability of the foam. These effects arise in compositions according to the invention in particular through combination with cationic and/or amphoteric polymers.

In alkyl and/or alkenyl oligoglycosides, at least one hydroxyl group is preferably replaced in at least one of the residues G by —O—C₁₋₁₂-alkenyl-COOM, —OSO₃M, —OP(O)(OM)₂ or —O—CH₂—CH₂—SO₃M with M=H, alkali metal, NH₄ or one of the above-stated counterions such as Zn, Mg or alkanolammonium.

Particularly preferably, an alkyl oligoglycoside carboxylate is used, in which —O—C₁₋₁₂-alkylene-COOM means —O(CH₂—)ₙCOOM with M=H, Na or K and n=1 to 3. The residue is particularly preferably O—CH₂—COONa.

Particularly preferably, an alkyl oligoglycoside carboxylate is used, in which the alkyl residue is a lauryl residue. Especial preference is given to a lauryl glucoside carboxylate, as is obtainable from Cognis Deutschland as Plantapon® LCG.

In alkyl glycosides of the general formula (I), the glycoside units G are preferably derived from aldoses or ketoses.

The reducing saccharides, the aldoses, are preferably used due to their better reactivity. Among aldoses, glucose may in particular be considered due to its ready obtainability and industrial availability. The alkyl glycosides particularly preferably used as starting materials are therefore alkyl glucosides.

The index value p in the general formula (I) indicates the degree of oligomerization, i.e. the distribution of mono- and oligoglycosides, and denotes a number between 1 and 10. While p must always be integral in a given compound and in this case may primarily assume the values p=1 to 6, the value p for a specific alkyl glycoside is a calculated value determined by analysis and is usually a fractional number. Preferably, alkyl glycosides with an average degree of oligomerization p of 1.1 to 3.0 are used. Particular preference is given to those alkyl glycosides whose degree of oligomerization is less than 1.5 and in particular is between 1.1 and 1.4.

The alkyl residue R is derived from primary alcohols having 6 to 22, preferably 12 to 18 carbon atoms. Typical examples are caproic alcohol, caprylic alcohol, capric alcohol, lauryl alcohol, myristyl alcohol, cetyl alcohol, stearyl alcohol and behenyl alcohol, and technical fractions, which may also contain proportions of unsaturated alcohols in addition to the stated saturated alcohols and which are obtained on the basis of natural fats and oils, for example palm oil, palm kernel oil, coconut oil or beef fat. The use of technical coconut alcohol is here particularly preferred.

In addition to the stated fatty alcohols, the alkyl glycosides may also be derived from synthetic primary alcohols having 6 to 22 carbon atoms, in particular “oxo alcohols”, which have a proportion of from 5 to 40 wt. % branched isomers.

Particularly preferred alkyl residues are those having 8/10, 12/14, 8 to 16, 12 to 16 or 16 to 18 C atoms. Mixtures of alkyl residues are obtained during production starting from natural fats and oils or mineral oils.

Methods of producing these alkyl glycosides have long been known to a person skilled in the art.

The production of alkyl or alkenyl oligoglycoside carboxylates, phosphates, sulfates or isethionates may proceed according to known methods. Carboxylate production proceeds for example by reacting the alkyl oligoglycosides with salts of chlorocarboxylic acids in the presence of bases. For example, the reaction may be carried out with 2-chloroacetic acid sodium salt in the presence of NaOH. During the reaction, both the hydroxyl groups in the ring and the —CH₂—OH group may be reacted. The degree of conversion is dependent, inter alia, on the stoichiometry of the products used. Preferably, the alkyl oligoglycosides are reacted at least on the —CH₂—OH group, one or more of the hydroxyl groups located on the ring optionally being reacted on average.

Further hydroxyl groups may also be etherified, for example.

The production of isethionates likewise proceeds using known methods of the prior art. It is additionally known that the products may be used for hair care and personal hygiene. In particular, aqueous detergent mixtures are described, which contain alkyl oligoglycoside isethionates and for example further anionic surfactants.

The production of sulfates likewise proceeds using known methods. In addition, mixtures of APG sulfates have been described which comprise inter alia alkyl sulfates or alkyl ether sulfates as well as further constituents. It has been stated that the surfactant mixtures may be used in products which serve to wash, dye, wave or rinse hair.

The production of sulfates likewise proceeds according to prior art. For example, the corresponding alkyl glycoside may be reacted with gaseous sulfur trioxide or with sulfuric acid, followed by neutralization. Cosmetic and pharmaceutical preparations containing alkyl oligoglycoside sulfates are likewise known.

Finally, detergent mixtures of alkyl oligoglycoside sulfates and alkyl ether phosphates have been described, which may used for example in hair rinses, hair dyes or hair waving agents.

The mild anionic surfactants used according to the invention and particularly preferably the alkyl and/or alkenyl oligoglycoside carboxylates, sulfates, phosphates and/or isoethionates are used in a quantity of from 0.1 to 25 wt. %, particularly preferably 0.1 to 15 wt. % and very particularly preferably in a quantity of from 0.5 to 10.0 wt. %.

The mild anionic surfactants used and particularly preferably the alkyl and/or alkenyl oligoglycoside carboxylates, sulfates, phosphates and/or isoethionates may wholly or partially replace the conventional anionic surfactants in these agents. Thus, the mild anionic surfactants according to the invention may be used as the sole anionic surfactant in the agents, or mixtures of these mild anionic surfactants with one another or with further conventional anionic surfactants may be used. These conventional anionic surfactants will be explained in greater detail at a later point. For example, the mild anionic surfactants and further anionic surfactants may be present in a weight ratio in the range from 5:0.05 to 1:2, particularly preferably 3:0.5 to 1:2, in particular 2.5:0.5 to 1:1.5 and most preferably 1.5:1 to 1:1.5.

Those surface-active compounds which bear at least one quaternary ammonium group and at least one —COO⁽⁻⁾ or —SO₃⁽⁻⁾ group on each molecule are designated zwitterionic surfactants (E2). Particularly suitable zwitterionic surfactants are “betaines” such as N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazolines having in each case 8 to 18 C atoms in the alkyl or acyl group and cocoacylaminoethylhydroxyethylcarboxymethyl glycinate. One preferred zwitterionic surfactant is the fatty acid amide derivative known by the INCI name Cocamidopropyl Betaine.

Ampholytic surfactants (E3) are taken to mean those surface-active compounds which, in addition to a C₈-C₂₄ alkyl or acyl group, contain at least one free amino group and at least one —COOH or —SO₃H group per molecule and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropyl-glycines, N-alkyltaurines, N-alkylsarcosines, 2-alkylaminopropionic acids and alkylaminoacetic acids having in each case approx. 8 to 24 C atoms in the alkyl group. Typical examples of amphoteric or zwitterionic surfactants are alkylbetaines, alkylamidobetaines, aminopropionates, aminoglycinates, imidazolinium betaines and sulfobetaines.

Particularly preferred ampholytic surfactants are N-cocoalkyl aminopropionate, cocoacylaminoethyl aminopropionate and C₁₂₋₁₈ acyl sarcosine.

Nonionic surfactants (E4) contain as hydrophilic group for example a polyol group, a polyalkylene glycol ether group or a combination of a polyol group and polyglycol ether group. Such compounds are for example:

addition products of 2 to 50 mol of ethylene oxide and/or 0 to 5 mol of propylene oxide onto linear and branched fatty alcohols having 6 to 30 C atoms, fatty alcohol polyglycol ethers or fatty alcohol polypropylene glycol ethers or mixed fatty alcohol polyethers,
addition products of 2 to 50 mol of ethylene oxide and/or 0 to 5 mol of propylene oxide onto linear and branched fatty acids having 6 to 30 C atoms, fatty acid polyglycol ethers or fatty acid polypropylene glycol ethers or mixed fatty acid polyethers, addition products of 2 to 50 mol of ethylene oxide and/or 0 to 5 mol of propylene oxide onto linear and branched alkylphenols having 8 to 15 C atoms in the alkyl group, alkylphenol polyglycol ethers or alkyl polypropylene glycol ethers, or mixed alkylphenol polyethers, addition products, end group-terminated with a methyl or C₂-C₆ alkyl residue, of 2 to 50 mol of ethylene oxide and/or 0 to 5 mol of propylene oxide onto linear and branched fatty alcohols having 8 to 30 C atoms, onto fatty acids having 8 to 30 C atoms and onto alkylphenols having 8 to 15 C atoms in the alkyl group, such as for example the grades obtainable under the commercial names Dehydrol® LS, Dehydrol® LT (Cognis),
C₁₂-C₃₀ fatty acid mono- and diesters of addition products of 1 to 30 mol of ethylene oxide onto glycerol, addition products of 5 to 60 mol of ethylene oxide onto castor oil and hardened castor oil, polyol fatty acid esters, such as for example the commercial product Hydagen® HSP (Cognis) or Sovermol® grades (Cognis), alkoxylated triglycerides, alkoxylated fatty acid alkyl esters of the formula (E4-I)

R¹CO—(OCH₂CHR²)_{w}OR³ (E4-I)

in which R¹CO denotes a linear or branched, saturated and/or unsaturated acyl residue having 6 to 22 carbon atoms, R² denotes hydrogen or methyl, R³ denotes linear or branched alkyl residues having 1 to 4 carbon atoms and w denotes numbers from 1 to 20,
amine oxides, hydroxy mixed ethers, as described for example in DE-OS 19738866, sorbitan fatty acid esters and addition products of ethylene oxide onto sorbitan fatty acid esters such as for example polysorbates, sugar fatty acid esters and addition products of ethylene oxide onto sugar fatty acid esters, addition products of ethylene oxide onto fatty acid alkanolamides and fatty amines, sugar surfactants of the alkyl and alkenyl oligoglycoside type according to formula (E4-II),

R⁴O-[G]ₚ (E4-II)

in which R⁴ denotes an alkyl or alkenyl residue having 4 to 22 carbon atoms, G denotes a sugar residue having 5 or 6 carbon atoms and p denotes numbers from 1 to 10. They may be obtained in accordance with the relevant methods of preparative organic chemistry. Reference is made, representatively of the comprehensive literature, to the review article by Biermann et al. in Starch/Stärke 45, 281 (1993), B. Salka in Cosm. Toil. 108, 89 (1993) and J. Kahre et al. in SÖFW-Journal, issue 8, 598 (1995).
Alkyl and alkenyl oligoglycosides may be derived from aldoses or ketoses having 5 or 6 carbon atoms, preferably from glucose. Preferred alkyl and/or alkenyl oligoglycosides are thus alkyl and/or alkenyl oligoglucosides. The index value p in the general formula (E4-II) indicates the degree of oligomerization (DP), i.e. the distribution of mono- and oligoglycosides, and denotes a number between 1 and 10. While p must always be integral in the individual molecule and in this case may primarily assume the values p=1 to 6, the value p for a specific alkyl oligoglycoside is a calculated value determined by analysis and is usually a fractional number. Alkyl and/or alkenyl oligoglycosides having an average degree of oligomerization p of 1.1 to 3.0 are preferably used. From an applicational standpoint, preferred alkyl and/or alkenyl oligoglycosides are those whose degree of oligomerization is less than 1.7 and in particular is between 1.2 and 1.4. The alkyl or alkenyl residue R⁴ may be derived from primary alcohols having 4 to 11, preferably 8 to 10 carbon atoms. Typical examples are butanol, caproic alcohol, caprylic alcohol, capric alcohol and undecyl alcohol and the technical mixtures thereof, as are, for example, obtained from the hydrogenation of technical fatty acid methyl esters or in the course of hydrogenation of aldehydes from Roelen's oxo synthesis. Preferred alkyl oligoglucosides are those of a C₈-C₁₀ chain length (DP=1 to 3) which occur as forerunnings in the distillative separation of technical C₈-C₁₈ coconut fatty alcohol and may be contaminated with a proportion of less than 6 wt. % of C₁₋₂ alcohol and alkyl oligoglucosides based on technical C_{9/11} oxo alcohols (DP=1 to 3). The alkyl or alkenyl residue R¹⁵ may furthermore also be derived from primary alcohols having 12 to 22, preferably 12 to 14 carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol and the technical mixtures thereof, which may be obtained as described above. Preferred alkyl oligoglucosides are those based on hardened C_{12/14} coconut alcohol with a DP of 1 to 3.
Sugar surfactants of the fatty acid N-alkyl polyhydroxyalkylamide type, a nonionic surfactant of the formula (E4-III),

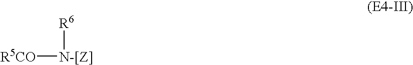

in which R⁵CO denotes an aliphatic acyl residue having 6 to 22 carbon atoms, R⁶ denotes hydrogen, an alkyl or hydroxyalkyl residue having 1 to 4 carbon atoms and [Z] denotes a linear or branched polyhydroxyalkyl residue having 3 to 12 carbon atoms and 3 to 10 hydroxyl groups. The fatty acid N-alkyl polyhydroxyalkylamides comprise known substances which may conventionally be obtained by reductive amination of a reducing sugar with ammonia, an alkylamine or an alkanolamine and subsequent acylation with a fatty acid, a fatty acid alkyl ester or a fatty acid chloride. Reference is made to US patents U.S. Pat. No. 1,985,424, U.S. Pat. No. 2,016,962 and U.S. Pat. No. 2,703,798 and to international patent application WO 92/06984 with regard to methods for the production thereof. A review of this topic by H. Kelkenberg may be found in Tens. Surf. Det. 25, 8 (1988). The fatty acid N-alkyl polyhydroxyalkylamides are preferably derived from reducing sugars having 5 or 6 carbon atoms, in particular from glucose. The preferred fatty acid N-alkyl polyhydroxyalkylamides are therefore fatty acid N-alkyl glucamides, as represented by the formula (E4-IV):

R⁷CO—(NR⁸)—CH₂—[CH(OH)]₄—CH₂OH (E4-IV)

Preferably, the fatty acid-N-alkyl polyhydroxyalkylamides take the form of glucamides of the formula (E4-IV), in which R⁸ denotes hydrogen or an alkyl group and R⁷CO denotes the acyl residue of caproic acid, caprylic acid, capric acid, lauric acid, myristic acid, palmitic acid, palmoleic acid, stearic acid, isostearic acid, oleic acid, elaidic acid, petroselinic acid, linoleic acid, linolenic acid, arachidic acid, gadoleic acid, behenic acid or erucic acid or the technical mixtures thereof. Particularly preferred fatty acid N-alkyl glucamides of the formula (E4-IV) are those which are obtained by reductive amination of glucose with methylamine and subsequent acylation with lauric acid or C_{12/14} coconut fatty acid or a corresponding derivative. The polyhydroxyalkylamides may also be derived from maltose and palatinose.

Sugar surfactants may preferably be contained in the agents used according to the invention in quantities of 0.1-20 wt. %, relative to the total agent. Quantities of 0.5-15 wt. % are preferred, and quantities of 0.5-7.5 wt. % are very particularly preferred.

Further typical examples of nonionic surfactants are fatty acid amide polyglycol ethers, fatty amine polyglycol ethers, mixed ethers or mixed formals, protein hydrolyzates (in particular wheat-based plant products) and polysorbates.

Preferred nonionic surfactants have proved to be alkylene oxide addition products onto saturated linear fatty alcohols and fatty acids with in each case 2 to 30 mol of ethylene oxide per mol of fatty alcohol or fatty acid respectively and sugar surfactants. Preparations having excellent properties are likewise obtained if they contain fatty acid esters of ethoxylated glycerol as the nonionic surfactants.

These compounds are characterized by the following parameters. The alkyl residue R contains 6 to 22 carbon atoms and may be both linear and branched. Primary linear aliphatic residues and those methyl-branched in position 2 are preferred. Such alkyl residues are for example 1-octyl, 1-decyl, 1-lauryl, 1-myristyl, 1-cetyl and 1-stearyl. 1-Octyl, 1-decyl, 1-lauryl, 1-myristyl are particularly preferred. When “oxo alcohols” are used as starting materials, compounds having an uneven number of carbon atoms in the alkyl chain predominate.

The compounds with alkyl groups used as surfactant may in each case comprise uniform substances. It is, however, generally preferred to start from native plant or animal raw materials when producing these substances, such that mixtures of substances having a differing alkyl chain length depending on the particular raw material are obtained.

The surfactants which are addition products of ethylene and/or propylene oxide onto fatty alcohols or derivatives of these addition products may be used both as products with a “normal” homolog distribution and as products with a narrow homolog distribution. A “normal” homolog distribution is here taken to mean mixtures of homologs which are obtained on reacting fatty alcohol and alkylene oxide using alkali metals, alkali metal hydroxides or alkali metal alkoxides as catalysts. Narrow homolog distributions, in contrast, are obtained if hydrotalcite, alkaline earth metal salts of ether carboxylic acids, alkaline earth metal oxides, hydroxides or alkoxides are for example used as catalysts. It may be preferred to use products with a narrow homolog distribution.

As additives for further improving the creaminess of the foam and of the skin feel during and after application, nonionic surfactants have also proven useful, and additional use thereof for production of the compositions according to the invention may be recommended: particular preference is therefore given to compositions according to the invention with an additional content of 0.1-20 wt. % of nonionic surfactants with an HLB value of 2-18. Such products may be produced by addition of ethylene oxide onto for example fatty alcohols having 6-30 C atoms, onto fatty acids having 6-30 C atoms or onto glycerol or sorbitan fatty acid partial esters based on C₁₂-C₁₈ fatty acids or onto fatty acid alkanolamides. The HLB value means the proportion of hydrophilic groups, for example glycol ether or polyol groups relative to the total molecule and it is calculated in accordance with the relationship

HLB=1/5×(100 wt. % L),

wt. % L being the proportion by weight of lipophilic groups, thus for example an alkyl or acyl group with 6-30 C atoms in the surfactant molecule.

Cationic surfactants (E5) form the final group of surfactants. Cationic surfactants are distinguished as part of the active ingredient complex according to the invention in that, like amphoteric and zwitterionic surfactants, they contribute to a markedly improved cosmetic appearance of skin and hair. The cationic charge ensures a good bond to the more probably negatively charged surfaces in particular of damaged hair or stressed skin. Active ingredients with a relatively hydrophobic structure may in turn be attached to a greater extent to the long fatty residues of these molecule structures. In this way, increased deposition of care substances on the surface of skin and hair is brought about overall. The hair is for example more readily combable, more readily stylable and glossier as well as being more pleasant to handle both in the dry and in the wet state.

Cationic surfactants (E5) are derived in general from ammonium ions and have a structure (NR¹R²R³R⁴)⁺ with a correspondingly negatively charged counterion. Such cationic ammonium compounds are very well known to a person skilled in the art. Further cationic surfactants are for example ester quats or imidazolium compounds. According to the invention, particular preference is given to the use of cationic surfactants (E5) of the type including quaternary ammonium compounds, ester quats, imidazolines and amidoamines. Preferred quaternary ammonium compounds are ammonium halides, in particular chlorides and bromides, such as alkyltrimethylammonium chlorides, dialkyldimethylammonium chlorides and trialkylmethylammonium chlorides, for example cetyltrimethylammonium chloride, stearyltrimethylammonium chloride, distearyldimethylammonium chloride, lauryldimethylammonium chloride, lauryldimethylbenzylammonium chloride and tricetylmethylammonium chloride, and the imidazolinium compounds known under the INCI names Quaternium-27 and Quaternium-83. The long alkyl chains of the above-stated surfactants preferably comprise 8 to 30 carbon atoms. Typical examples of cationic surfactants are quaternary ammonium compounds and ester quats, in particular quaternized fatty acid trialkanolamine ester salts.

Particularly preference may be given according to the invention to the use of cationic compounds with behenyl residues, in particular the substances known under the name behentrimonium chloride or bromide (docosanyltrimethylammonium chloride or bromide). Other preferred QACs comprise at least two behenyl residues. These substances are commercially available for example under the name Genamin® KDMP (Clariant).

Ester quats are known substances which contain both at least one ester function and at least one quaternary ammonium group as a structural element. Preferred ester quats are quaternized ester salts of fatty acids with triethanolamine, quaternized ester salts of fatty acids with diethanolalkylamines and quaternized ester salts of fatty acids with 1,2-dihydroxypropyldialkylamines. Such products are distributed, for example, under the trademarks Stepantex®, Dehyquart® and Armocare®. The products Armocare® VGH-70, an N,N-bis(2-palmitoyloxyethyl)dimethylammonium chloride, and Dehyquart® F-75, Dehyquart® C-4046, Dehyquart® L80 and Dehyquart® AU-35 are examples of such ester quats.

The agents according to the invention may contain, as further cationic surfactants, at least one quaternary imidazoline compound, i.e. a compound which comprises a positively charged imidazoline ring. The formula (E5-V) illustrated below shows the structure of these compounds.

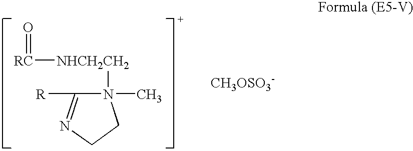

The residues R mutually independently in each case denote a saturated or unsaturated, linear or branched hydrocarbon residue with a chain length of 8 to 30 carbon atoms. The preferred compounds of the formula (E5-V) in each case contain the identical hydrocarbon residue for R. The chain length of the residues R is preferably 12 carbon atoms. Particularly preferred compounds are those with a chain length of at least 16 carbon atoms, those with at least 20 carbon atoms being particularly preferred. A very particularly preferred compound of the formula I has a chain length of 21 carbon atoms. A commercial product of this chain length is known for example under the name Quaternium-91. In the formula (E5-V) the counterion is methosulfate. According to the invention, however, the counterions may also be halides such as chloride, fluoride, bromide or also phosphates.

The imidazolines of the formula (E5-V) are contained in the compositions according to the invention in quantities of 0.01 to 20 wt. %, preferably in quantities of 0.05 to 10 wt. % and very particularly preferably in quantities of 0.1 to 7.5 wt. %. The very best results are obtained with quantities of 0.1 to 5 wt. % in each case relative to the total composition of the respective agent.

The alkylamidoamines are conventionally produced by amidating natural or synthetic fatty acids and fatty acid cuts with dialkylaminoamines. One compound from this group of substances which is particularly suitable according to the invention is stearamidopropyldimethylamine which is commercially available under the name Tegoamid® S 18. The alkylamidoamines may both be present as such and be converted into a quaternary compound in the composition by protonation in an appropriately acidic solution, but they may also of course be used as a permanently quaternary compound in the compositions according to the invention. Examples of permanently quaternized amidoamines are for example the raw materials with the tradenames Rewoquat® UTM 50, Lanoquat® DES-50 or Empigen CSC.

The agents used according to the invention preferably contain the cationic surfactants (E5) in quantities of 0.05 to 10 wt. %, relative to the total agent. Quantities of 0.1 to 5 wt. % are particularly preferred.

Cationic, zwitterionic and/or amphoteric surfactants and mixtures thereof may be preferred according to the invention. Anionic surfactants are used in particular if the compositions according to the invention are to be used as shampoos.

The surfactants (E) are used in quantities of 0.05-45 wt. %, preferably of 0.1-30 wt. % and very particularly preferably of 0.5-25 wt. %, relative to the total agent used according to the invention.

Emulsifiers bring about the formation of water- or oil-resistant adsorption layers at the phase interface, which protect the dispersed droplets from coalescence and so stabilize the emulsion. Emulsifiers, like surfactants, are thus made up of a hydrophobic and a hydrophilic molecular moiety. Hydrophilic emulsifiers preferably form O/W emulsions while hydrophobic emulsifiers preferably form W/O emulsions. An emulsion is taken to mean a droplet distribution (dispersion) of one liquid in another liquid with the input of energy to create stabilizing phase interfaces by means of surfactants. The selection of these emulsifying surfactants or emulsifiers is here determined on the basis of the substances to be dispersed and the particular external phase and the fineness of the emulsion. Further definitions and properties of emulsifiers may be found in “H.-D. Dörfler, Grenzflächen- und Kolloidchemie [interfacial and colloid chemistry], VCH Verlagsgesellschaft mbH, Weinheim, 1994”. Examples of emulsifiers which may be used according to the invention are: addition products of 4 to 30 mol of ethylene oxide and/or 0 to 5 mol of propylene oxide onto linear fatty alcohols having 8 to 22 C atoms, onto fatty acids having 12 to 22 C atoms and onto alkylphenols having 8 to 15 C atoms in the alkyl group, C₁₂-C₂₂ fatty acid mono- and diesters of addition products of 1 to 30 mol of ethylene oxide onto polyols having 3 to 6 carbon atoms, in particular onto glycerol, ethylene oxide and polyglycerol addition products onto methyl glucoside fatty acid esters, fatty acid alkanolamides and fatty acid glucamides, C₈-C₂₂ alkyl mono- and oligoglycosides and the ethoxylated analogs thereof, with degrees of oligomerization of 1.1 to 5, in particular of 1.2 to 2.0, and glucose as the sugar component being preferred, mixtures of alkyl (oligo)glucosides and fatty alcohols, for example the commercially available product Montanov® 68, addition products of 5 to 60 mol of ethylene oxide onto castor oil and hardened castor oil, partial esters of polyols having 3-6 carbon atoms with saturated fatty acids having 8 to 22 C atoms, sterols. Sterols are taken to be a group of steroids which bear a hydroxyl group on C atom 3 of the steroid skeleton and may be isolated both from animal tissue (zoosterols) and from vegetable fats (phytosterols). Examples of zoosterols are cholesterol and lanosterol. Examples of suitable phytosterols are ergosterol, stigmasterol and sitosterol. Sterols are also isolated from fungi and yeasts, these being known as mycosterols. phospholipids. These are primarily taken to mean glucose phospholipids which are for example obtained as lecithins, or phosphatidylcholines for example from egg yolk or plant seeds (for example soy beans). Fatty acid esters of sugars and sugar alcohols, such as sorbitol, polyglycerols and polyglycerol derivatives such as for example polyglycerol poly-12-hydroxystearate (commercial product Dehymuls® PGPH), linear and branched fatty acids having 8 to 30 C atoms and the Na, K, ammonium, Ca, Mg and Zn salts thereof.

The addition of a per se known emulsifier of the water-in-oil type in a quantity of approx. 1-5 wt. % has proven particularly advantageous. This comprises a mixed ester, which is a condensation product of a pentaerythritol difatty acid ester and a citric acid difatty alcohol ester, as is described in greater detail in DE-PS 11 65 574. Through the addition of such mixed esters, a particularly creamy, fine-bubble foam and a pleasant skin feel are achieved when the body cleansing agent is applied.

The agents according to the invention preferably contain the emulsifiers in quantities of 0.1-25 wt. %, in particular of 0.5-15 wt. %, relative to the total agent.

The compositions according to the invention may preferably contain at least one nonionogenic emulsifier with an HLB value of 8 to 18 in accordance with the definitions provided in Römpp-Lexikon Chemie [Römpp lexicon of chemistry] (eds. J. Falbe, M. Regitz), 10th edition, Georg Thieme Verlag Stuttgart, New York, (1997), page 1764. Nonionogenic emulsifiers with an HLB value of 10-15 may be particularly preferred according to the invention. Furthermore, it is preferred according to the invention for the mild anionic surfactants to be used together with other surfactants and emulsifiers known as mild or particularly mild. The criteria for this are the above-described test models. According to these test methods the following surfactants and emulsifiers have proven to be mild to particularly mild and are particularly preferred according to the invention:

esters of tartaric acid and citric acid with alcohols, which are addition products of approx. 2-15 molecules of ethylene oxide and/or propylene oxide onto fatty alcohols having 8 to 22 C atoms,
zwitterionic surfactants (E2),
ampholytic surfactants (E3),
sugar surfactants of the alkyl or alkenyl oligoglycoside type of the formula (E4-II),
sugar surfactants of the fatty acid N-alkyl polyhydroxyalkylamide type according to the formula (E4-III),
addition products of 5 to 60 mol of ethylene oxide onto castor oil and hardened castor oil,
polyol fatty acid esters, such as for example the commercial product Hydagen® HSP (Cognis) or Sovermol grades (Cognis),
amine oxides,
hydroxy mixed ethers, as are described for example in DE-OS 19738866, sorbitan fatty acid esters and addition products of ethylene oxide onto sorbitan fatty acid esters such as for example polysorbates,
sugar fatty acid esters and addition products of ethylene oxide onto sugar fatty acid esters, addition products of ethylene oxide onto fatty acid alkanolamides and fatty amines,
sugar surfactants of the alkyl and alkenyl oligoglycoside type of the formula (E4-II), ester quats,
alkylamidoamines and quaternized alkylamidoamines.
C₈-C₂₂ alkyl mono- and oligoglycosides and the ethoxylated analogues thereof, with degrees of oligomerization of 1.1 to 5, in particular of 1.2 to 2.0, and glucose as the sugar component being preferred,
mixtures of alkyl (oligo)glucosides and fatty alcohols, for example the commercially available product Montanov® 68,
addition products of 5 to 60 mol of ethylene oxide onto castor oil and hardened castor oil,
partial esters of polyols having 3-6 carbon atoms with saturated fatty acids having 8 to 22 C atoms,
sterols. Sterols are taken to be a group of steroids which bear a hydroxyl group on C atom 3 of the steroid skeleton and may be isolated both from animal tissue (zoosterols) and from vegetable fats (phytosterols). Examples of zoosterols are cholesterol and lanosterol. Examples of suitable phytosterols are ergosterol, stigmasterol and sitosterol. Sterols are also isolated from fungi and yeasts, these being known as mycosterols.
phospholipids. These are primarily taken to mean glucose phospholipids which are for example obtained as lecithins, or phosphatidylcholines for example from egg yolk or plant seeds (for example soy beans). fatty acid esters of sugars and sugar alcohols, such as sorbitol,
polyglycerols and polyglycerol derivatives such as for example polyglycerol poly-12-hydroxystearate (commercial product Dehymuls® PGPH).

In the case preferred according to the invention, where it is intended to improve the compatibility of the cosmetic compositions, the particularly mild surfactants and emulsifiers are preferably used in the compositions. In such cases, the alkyl sulfates and/or alkyl ether sulfates are used in quantities of below 8 wt. %, preferably less than 5 wt. % and particularly preferably less than 2.5 wt. %. Very particularly preferably, these compositions are free of alkyl sulfates and/or alkyl ether sulfates. In this context, “free of” means that in no way are these constituents additionally used. It is possible, however, that they enter the composition by way of other constituents, such as for example through the use of silicone emulsions. Preferably, therefore, “free of” also means less than 0.5 wt. %, particularly preferably less than 0.1 wt. %. Furthermore, “free of” does not mean in this context that the compositions according to the invention must not contain any fatty alcohol ether sulfates with a narrow homolog distribution. Rather, this is an exception, since these anionic surfactants may be classed as mild anionic surfactants. According to the invention, however, greatest preference is given to the exclusive use of mild surfactants. In such cases, should “non-mild surfactants” get into the compositions according to the invention as an admixture to other constituents, the above-stated quantities apply to these admixtures.

It is known that oil-in-water-emulsions, hereinafter stated as O/W emulsions, which are produced and stabilized with non-ionogenic emulsifiers, undergo phase inversion on heating, i.e. at relatively elevated temperatures the outer, aqueous phase may become the inner phase. As a rule, this process is reversible, i.e. the original emulsion type re-forms on cooling. It is also known that the situation of phase inversion temperature (PIT) is dependent on many factors, for example on the type and phase volume of the oil component, on the hydrophilicity and structure of the emulsifier or the composition of the emulsifier system. It is additionally known that emulsions which are produced at or a little below the phase inversion temperature are distinguished by particular stability and fineness, while those which are produced above the phase inversion temperature are less finely divided. Emulsions which suffer phase inversion at a particular temperature are known as PIT emulsions. These PIT emulsions may be preferred according to the invention because they contain less emulsifier, due to the exactly sufficient amount of emulsifier, than conventional non-PIT emulsions. They are therefore not only particularly cost-effective, but also particularly mild and gentle on the skin and hair. If ionic surfactants are also used as emulsifiers in the PIT emulsions, then these are particularly preferably only added to the PIT emulsion after production of the PIT emulsion during the cooling process.

The synergists of the compositions according to the invention include anionic and nonionic polymers. Polymers are used in cosmetic compositions for a plurality of reasons.

A few examples of particularly preferred polymers are described below. The polymers usable according to the invention may be distinguished on the basis of the charges of the polymers and/or on the basis of their particularly marked applicational properties. The expression “particularly marked properties” reflects the fact that polymers generally combine a plurality of properties in one molecule. Frequently, however, one of the properties is particularly distinct and is decisive when it comes to selecting precisely this polymer.

The anionic polymers (G2) are anionic polymers which comprise carboxylate and/or sulfonate groups. Examples of anionic monomers of which such polymers may consist are acrylic acid, methacrylic acid, crotonic acid, maleic anhydride and 2-acrylamido-2-methylpropanesulfonic acid. In this case, the acidic groups may be present wholly or in part as a sodium, potassium, ammonium, mono- or triethanolammonium salt. 2-Acrylamido-2-methylpropanesulfonic acid and acrylic acid are preferred monomers.

Anionic polymers which have proven very particularly effective are those which contain as sole or co-monomer 2-acrylamido-2-methylpropanesulfonic acid, wherein the sulfonic acid group may be present wholly or in part as a sodium, potassium, ammonium, mono- or triethanolammonium salt.

The homopolymer of 2-acrylamido-2-methylpropanesulfonic acid is particularly preferred, and is commercially available for example under the name Rheothik® 11-80.

Within this embodiment it may be preferable to use copolymers of at least one anionic monomer and at least one nonionogenic monomer. With regard to anionic monomers, reference is made to the above-listed substances. Preferred nonionogenic monomers are acrylamide, methacrylamide, acrylic acid esters, methacrylic acid esters, vinylpyrrolidone, vinyl ethers and vinyl esters.

Preferred anionic copolymers are acrylic acid-acrylamide copolymers and in particular polyacrylamide copolymers with monomers containing sulfonic acid groups. A particularly preferred anionic copolymer consists of 70 to 55 mol % acrylamide and 30 to 45 mol % 2-acrylamido-2-methylpropanesulfonic acid, the sulfonic acid group being present wholly or in part as a sodium, potassium, ammonium, mono- or triethanolammonium salt. This copolymer may also be present in crosslinked form, wherein polyolefinically unsaturated compounds such as tetraallyloxyethane, allyl sucrose, allyl pentaerythritol and methylenebisacrylamide are preferably used as the crosslinking agents. Such a polymer is contained in the commercial product Sepigel® 305 from SEPPIC. Use of this compound, which contains in addition to the polymer component a hydrocarbon mixture (C₁₃-C₁₄ isoparaffin) and a non-ionogenic emulsifier (Laureth-7), has proven particularly advantageous for the purposes of the teaching according to the invention.

The sodium acryloyldimethyl taurate copolymers distributed under the name Simulgel® 600 as a compound with isohexadecane and polysorbate-80 have also proven particularly effective according to the invention.

Anionic homopolymers which are likewise preferred are uncrosslinked and crosslinked polyacrylic acids. In this case, allyl ethers of pentaerythritol, of sucrose and of propylene may be preferred crosslinking agents. Such compounds are commercially available for example under the tradename Carbopol®.

Copolymers of maleic anhydride and methyl vinyl ether, in particular those comprising crosslinks, are also color-preserving polymers. A maleic acid-methyl vinyl ether copolymer crosslinked with 1,9-decadiene is commercially available under the name Stabileze® QM.

The anionic polymers are contained in the agents according to the invention preferably in quantities of from 0.05 to 10 wt. %, relative to the total agent. Quantities of 0.1 to 5 wt. % are particularly preferred.

Polyurethanes are a further very particularly preferred group of polymers. The polyurethanes consist of at least two different types of monomer,

a compound (V1) with at least 2 active hydrogen atoms per molecule and

a di- or polyisocyanate (V2).

The compounds (V1) may for example be diols, triols, diamines, triamines, polyetherols and polyesterols. Compounds with more than 2 active hydrogen atoms are conventionally only used in small quantities in combination with a large excess of compounds with 2 active hydrogen atoms.

Examples of compounds (V1) are ethylene glycol, 1,2- and 1,3-propylene glycol, butylene glycols, di-, tri-, tetra- and polyethylene and -propylene glycols, copolymers of lower alkylene oxides such as ethylene oxide, propylene oxide and butylene oxide, ethylenediamine, propylenediamine, 1,4-diaminobutane, hexamethylenediamine and omega,omega-diamines based on long-chain alkanes or polyalkylene oxides.

Polyurethanes in which the compounds (V1) are diols, triols and polyetherols, may be preferred according to the invention. In particular, polyethylene glycols and polypropylene glycols with molar masses of between 200 and 3000, in particular of between 1600 and 2500, have proven particularly suitable in individual cases.

Polyesterols are conventionally obtained by modifying the compound (V1) with dicarboxylic acids such as phthalic acid, isophthalic acid and adipic acid.

Hexamethylene diisocyanate, 2,4- and 2,6-toluene diisocyanate, 4,4′-methylene di(phenyl isocyanate) and in particular isophorone diisocyanate are predominantly used as compounds (V2).

Furthermore, the polyurethanes according to the invention may additionally contain building blocks such as for example diamines as chain extenders and hydroxycarboxylic acids. Dialkylolcarboxylic acids such as for example dimethylolpropionic acid are particularly suitable hydroxycarboxylic acids. With regard to the further building blocks, there is no fundamental restriction as to whether they are nonionic, anionic or cationic building blocks.

For further information about the structure and production of polyurethanes, express reference is made to articles in the specialist review publications such as Römpps Chemie-Lexikon und Ullmanns Enzyklopädie der technischen Chemie.

Polyurethanes which have in many cases proven particularly suitable according to the invention may be distinguished as follows:

solely aliphatic groups in each molecule

no free isocyanate groups in each molecule

polyether and polyester polyurethanes

anionic groups in each molecule.

It has likewise proven advantageous in some cases for the polyurethane not to be dissolved in the system but rather stably dispersed.

Furthermore, it has proven advantageous for production of the agents according to the invention for the polyurethanes not to be mixed directly with the further components but rather to be introduced in the form of aqueous dispersions. Such dispersions conventionally comprise a solids content of approx. 20-50%, in particular approx. 35-45% and are also commercially obtainable.

A very particularly preferred polyurethane according to the invention is commercially available under the tradename Luviset® PUR (BASF).

In a further embodiment, the agents according to the invention may contain nonionogenic polymers (G4).

Suitable nonionogenic polymers are for example:

vinylpyrrolidone/vinyl ester copolymers, as are distributed for example under the tradename Luviskol® (BASF). Luviskol® VA 64 and Luviskol® VA 73, in each case vinylpyrrolidone/vinyl acetate copolymers, are likewise preferred nonionic polymers.
cellulose ethers, such as hydroxypropylcellulose, hydroxyethylcellulose and methylhydroxypropylcellulose, as are distributed for example under the tradenames Culminal® and Benecel® (AQUALON) and Natrosol® grades (Hercules).
starch and the derivatives thereof, in particular starch ethers, for example Structure® XL (National Starch), a multifunctional, salt-tolerant starch;
shellac,
polyvinylpyrrolidones, as are distributed for example under the tradename Luviskol® (BASF).
glycosidically substituted silicones.

The nonionic polymers are contained in the agents according to the invention preferably in quantities of from 0.05 to 10 wt. %, relative to the total agent. Quantities of 0.1 to 5 wt. % are particularly preferred.

The term polymer should be taken according to the invention likewise to mean specific preparations of polymers such as spherical polymer powders. Various methods are known for producing such microspheres from various monomers, for example by special polymerization methods or by dissolving the polymers in a solvent and spraying into a medium in which the solvent can evaporate or diffuse out of the particles. Such a method is known, for example, from EP 466 986 B1. Suitable polymers are for example polycarbonates, polyurethanes, polyacrylates, polyolefins, polyesters or polyamides. Particularly suitable such spherical polymer powders are those having a primary particle diameter of less than 1 μm. Such products based on a polymethacrylate copolymer are, for example, commercially available under the trademark Polytrap® Q5-6603 (Dow Corning). Other polymer powders, for example based on polyamides (Nylon 6, Nylon 12), are obtainable with a particle size of 2-10 μm (90%) and a specific surface area of approx. 10 m²/g under the tradename Orgasol® 2002 DU Nat Cos (Atochem S.A., Paris). Further spherical polymer powders which are suitable for the purpose according to the invention are, for example, polymethacrylates (Micropearl M from SEPPIC or Plastic Powder A from NIKKOL), styrene/divinylbenzene copolymers (Plastic Powder FP from NIKKOL), polyethylene and polypropylene powders (ACCUREL EP 400 from AKZO) or also silicone polymers (Silicone Powder X2-1605 from Dow Corning) or also spherical cellulose powders.

The above-described polymer powders are contained in the agents according to the invention preferably in quantities of from 0.05 to 10 wt. %, relative to the total agent. Quantities of 0.1 to 5 wt. % are particularly preferred.

Polymers may also be characterized independently of their chemical structure and charge according to their function in cosmetics. The description of the polymers according to their function in the agents according to the invention does not have to correspond with a value or meaning of these polymers. Instead, all the polymers should be regarded in principle as equivalent for use in the agents according to the invention, although some of these polymers may be preferred. If the polymers described hereinafter in accordance with their function in the compositions according to the invention are cationic and/or amphoteric, they are an essential component of the present composition. Differently charged or neutral polymers are optional constituents of the present invention.

Furthermore, some polymers are found in several descriptions of different effects due to the multifunctional nature of polymers. Polymers which may bring about a plurality of desired effects are consequently particularly preferred for use in the agents according to the invention.

Selection of the suitable polymer also depends on the use to which the composition according to the invention is to be put. Thus, for example, a film-forming cationic or amphoteric polymer is particularly preferably selected if the composition is to be used as a styling composition or setting preparation.

Since polymers are often multifunctional, their functions cannot always be clearly and unambiguously delimited from one another. This is true in particular of film-forming and setting polymers. Some film-forming polymers will nevertheless be described by way of example. It should however be explicitly pointed out at this point that both film-forming and setting polymers are essential for the purposes of the present invention. Since the two properties are also not wholly mutually independent, the term “setting polymers” should always be understood also to mean “film-forming polymers” and vice versa.

The preferred properties of film-forming polymers include film formation. Film-forming polymers should be understood to mean those polymers which, on drying, leave behind a continuous film on the skin, hair or nails. Such film formers may be used in the most varied of cosmetic products, such as for example face masks, make-up, hair setting preparations, hairsprays, hair gels, hair waxes, hair masks, shampoos or nail polishes. Preferred polymers are in particular those which exhibit sufficient solubility in alcohol or water/alcohol mixtures to be present in completely dissolved form in the agent according to the invention when used. Due to their marked film-forming properties, these polymers are particularly preferred in the agents according to the invention. The use of at least one of these polymers is therefore likewise very particularly preferred according to the invention. The film-forming polymers may be of synthetic or natural origin.

Film-forming polymers are further understood according to the invention to mean those polymers which are capable, when applied in a 0.01 to 20% aqueous, alcoholic or aqueous/alcoholic solution, of depositing a transparent polymer film on the hair. The film-forming polymers may be charged in any one of an anionic, amphoteric, nonionic, permanently cationic or temporarily cationic manner.

Suitable synthetic, film-forming, hair-setting polymers are homo- or copolymers synthesized from at least one of the following monomers: vinylpyrrolidone, vinylcaprolactam, vinyl esters such as for example vinyl acetate, vinyl alcohol, acrylamide, methacrylamide, alkyl- and dialkylacrylamide, alkyl- and dialkylmethacrylamide, alkyl acrylate, alkyl methacrylate, propylene glycol or ethylene glycol, the alkyl groups of these monomers preferably being C₁ to C₇ alkyl groups, particularly preferably C₁ to C₃ alkyl groups.

Suitable examples are homopolymers of vinylcaprolactam, vinylpyrrolidone or N-vinylformamide. Examples of further suitable synthetic film-forming, hair-setting polymers are copolymers of vinylpyrrolidone and vinyl acetate, terpolymers of vinylpyrrolidone, vinyl acetate and vinyl propionate, polyacrylamides, which are distributed for example by CHEM-Y, Emmerich under the tradename Akypomine® P 191, or by Seppic under the tradename Sepigel 305®; polyvinyl alcohols, which are sold for example by DuPont under the tradename Elvanol® or by Air Products under the tradename Vinol® 523/540 as well as polyethylene glycol/polypropylene glycol copolymers, which are sold, for example, by Union Carbide under the tradename Ucon®. Polyvinylpyrrolidone and polyvinylpyrrolidone/vinyl acetate copolymers are particularly preferred.

Examples of suitable natural film-forming polymers are cellulose derivatives, for example hydroxypropylcellulose with a molecular weight of 30,000 to 50,000 g/mol, which is distributed for example by Lehmann & Voss, Hamburg under the tradename Nisso SI®.

Examples of common film formers are Abies Balsamea (Balsam Canada) Resin, Acetylenediurea/Formaldehyde/Tosylamide Crosspolymer, Acrylamide/Ammonium Acrylate Copolymer, Acrylamides Copolymer, Acrylamides/DMAPA Acrylates/Methoxy PEG Methacrylate Copolymer, Acrylamide/Sodium Acrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/Behenyl Methacrylate/Dimethicone Methacrylate Copolymer, Acrylates/t-Butylacrylamide Copolymer, Acrylates Copolymer, Acrylates/Diacetoneacrylamide Copolymer, Acrylates/Dimethicone Copolymer, Acrylates/Dimethicone Methacrylate Copolymer, Acrylates/Dimethiconol Acrylate Copolymer, Acrylates/Dimethylaminoethyl Methacrylate Copolymer, Acrylates/Ethylhexyl Acrylate Copolymer, Acrylates/Ethylhexyl Acrylate/HEMA/Styrene Copolymer, Acrylates/Ethylhexyl Acrylate/Styrene Copolymer, Acrylates/Hydroxyesters Acrylates Copolymer, Acrylates/Lauryl Acrylate/Stearyl Acrylate/Ethylamine Oxide Methacrylate Copolymer, Acrylates/Octylacrylamide Copolymer, Acrylates/Propyl Trimethicone Methacrylate Copolymer, Acrylates/Stearyl Acrylate/Dimethicone Methacrylate Copolymer, Acrylates/Stearyl Acrylate/Ethylamine Oxide Methacrylate Copolymer, Acrylates/TDI/Trimethylolpropane Copolymer, Acrylates/VA Copolymer, Acrylates/VA Crosspolymer, Acrylates/VP Copolymer, Acrylates/VP/Dimethylaminoethyl Methacrylate/Diacetone Acrylamide/Hydroxypropyl Acrylate Copolymer, Acrylic Acid/Acrylonitrogens Copolymer, Adipic Acid/CHDM/MA/Neopentyl Glycol/Trimellitic Anhydride Copolymer, Adipic Acid/Diethylene Glycol/Glycerin Crosspolymer, Adipic Acid/Diethylenetriamine Copolymer, Adipic Acid/Dilinoleic Acid/Hexylene Glycol Copolymer, Adipic Acid/Dimethylaminohydroxypropyl Diethylenetriamine Copolymer, Adipic Acid/Epoxypropyl Diethylenetriamine Copolymer, Adipic Acid/Fumaric Acid/Phthalic Acid/Tricyclodecane Dimethanol Copolymer, Adipic Acid/Isophthalic Acid/Neopentyl Glycol/Trimethylolpropane Copolymer, Adipic Acid/Neopentyl Glycol/Trimellitic Anhydride Copolymer, Adipic Acid/PPG-10 Copolymer, Albumen, Allyl Stearate/VA Copolymer, Aloe Barbadensis Leaf Polysaccharides, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium Acrylates/Acrylonitrogens Copolymer, Ammonium Acrylates Copolymer, Ammonium Alginate, Ammonium Polyacrylate, Ammonium Styrene/Acrylates Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates Copolymer, AMP-Acrylates/Diacetoneacrylamide Copolymer, AMP-Acrylates/Dimethylaminoethylmethacrylate Copolymer, Astragalus Gummifer Gum, Avena Sativa (Oat) Kernel Protein, Behenyl Methacrylate/Perfluorooctylethyl Methacrylate Copolymer, Benzoguanamine/Formaldehyde/Melamine Crosspolymer, Benzoic Acid/Phthalic Anhydride/Pentaerythritol/Neopentyl Glycol/Palmitic Acid Copolymer, Bis-Hydrogenated Tallow Amine Dilinoleic Acid/Ethylenediamine Copolymer, Bis-PEG-15 Dimethicone/IPDI Copolymer, Bis-PPG-15 Dimethicone/IPDI Copolymer, Bis-Stearyl Dimethicone, Brassica Campestris/Aleurites Fordii Oil Copolymer, Butadiene/Acrylonitrile Copolymer, 1,4-Butandiol/Succinic Acid/Adipic Acid/HDI Copolymer, Butoxy Chitosan, Butyl Acrylate Crosspolymer, Butyl Acrylate/Ethylhexyl Methacrylate Copolymer, Butyl Acrylate/Hydroxyethyl Methacrylate Copolymer, Butyl Acrylate/Hydroxypropyl Dimethicone Acrylate Copolymer, Butyl Acrylate/Styrene Copolymer, Butylated Polyoxymethylene Urea, Butylated PVP, Butyl Benzoic Acid/Phthalic Anhydride/Trimethylolethane Copolymer, Butylene/Ethylene/Propylene Copolymer, Butyl Ester of Ethylene/MA Copolymer, Butyl Ester of PVM/MA Copolymer, Butylethylpropanediol Dimer Dilinoleate, Butyl Methacrylate/DMAPA Acrylates/Vinylacetamide Crosspolymer, C23-43 Acid Pentaerythritol Tetraester, Calcium Carboxymethyl Cellulose, Calcium Carrageenan, Calcium Potassium Carbomer, Calcium/Sodium PVM/MA Copolymer, C₅₋₆ Alkane/Cycloalkane/Terpene Copolymer, C30-45 Alkyl Dimethicone/Polycyclohexene Oxide Crosspolymer, C1-5 Alkyl Galactomannan, Candelilla Wax Hydrocarbons, Carboxybutyl Chitosan, Carboxymethyl Chitosan, Carboxymethyl Chitosan Succinamide, Carboxymethyl Dextran, Carboxymethyl Hydroxyethylcellulose, Castor Oil/IPDI Copolymer, Cellulose Acetate, Cellulose Acetate Butyrate, Cellulose Acetate Propionate, Cellulose Acetate Propionate Carboxylate, Cellulose Gum, Cetearyl Dimethicone/Vinyl Dimethicone Crosspolymer, Chitosan, Chitosan Adipate, Chitosan Ascorbate, Chitosan Formate, Chitosan Glycolate, Chitosan Lactate, Chitosan PCA, Chitosan Salicylate, Chitosan Succinamide, C₅₋₆ Olefin/C8-10 Naphtha Olefin Copolymer, Collodion, Copaifera Officinalis (Balsam Copaiba) Resin, Copal, Corn Starch/Acrylamide/Sodium Acrylate Copolymer, Corn Starch Modified, C₆₋₁₄ Perfluoroalkylethyl Acrylate/HEMA Copolymer, DEA-Styrene/Acrylates/DVB Copolymer, Dibutylhexyl IPDI, Didecyltetradecyl IPDI, Diethylene Glycolamine/Epichlorohydrin/piperazine Copolymer, Diethylhexyl IPDI, Diglycol/CHDM/Isophthalates/SIP Copolymer, Diglycol/Isophthalates/SIP Copolymer, Dihydroxyethyl Tallowamine/IPDI Copolymer, Dilinoleic Acid/Glycol Copolymer, Dilinoleic Acid/Sebacic Acid/piperazine/Ethylenediamine Copolymer, Dilinoleyl Alcohol/IPDI Copolymer, Dimethicone PEG-8 Polyacrylate, Dimethicone/Vinyltrimethylsiloxysilicate Crosspolymer, Dimethiconol/IPDI Copolymer, Dimethylamine/Ethylenediamine/Epichlorohydrin Copolymer, Dioctyidecyl IPDI, Dioctyldodecyl IPDI, Di-PPG-3 Myristyl Ether Adipate, Divinyldimethicone/Dimethicone Copolymer, Divinyldimethicone/Dimethicone Crosspolymer, DMAPA Acrylates/Acrylic Acid/Acrylonitrogens Copolymer, Dodecanedioic Acid/Cetearyl Alcohol/Glycol Copolymer, Ethylcellulose, Ethylene/Acrylic Acid Copolymer, Ethylene/Acrylic Acid/VA Copolymer, Ethylene/Calcium Acrylate Copolymer, Ethylene/MA Copolymer, Ethylene/Magnesium Acrylate Copolymer, Ethylene/Methacrylate Copolymer, Ethylene/Octene Copolymer, Ethylene/Propylene Copolymer, Ethylene/Sodium Acrylate Copolymer, Ethylene/VA Copolymer, Ethylene/Zinc Acrylate Copolymer, Ethyl Ester of PVM/MA Copolymer, Euphorbia Cerifera (Candelilla) Wax, Euphorbia Cerifera (Candelilla) Wax Extract, Fibroin/PEG-40/Sodium Acrylate Copolymer, Flexible Collodion, Formaldehyde/Melamine/Tosylamide Copolymer, Galactoarabinan, Glycereth-7 Hydroxystearate/IPDI Copolymer, Glycerin/MA/Rosin Acid Copolymer, Glycerin/Phthalic Acid Copolymer, Glycerin/Phthalic Acid Copolymer Castorate, Glycerin/Succinic Acid Copolymer Castorate, Glyceryl Diricinoleate/IPDI Copolymer, Glyceryl Polyacrylate, Glyceryl Polymethacrylate, Glyceryl Undecyl Dimethicone, Glycidyl C8-11 Acidate/Glycerin/Phthalic Anhydride Copolymer, Glycol Rosinate, Gutta Percha, Hexylene Glycol/Neopentyl Glycol/Adipic Acid/SMDI/DMPA Copolymer, Hydrogenated Brassica Campestris/Aleurites Fordii Oil Copolymer, Hydrogenated Caprylyl Olive Esters, Hydrogenated Cetyl Olive Esters, Hydrogenated Decyl Olive Esters, Hydrogenated Hexyl Olive Esters, Hydrogenated Lauryl Olive Esters, Hydrogenated Myristyl Olive Esters, Hydrogenated Rosin, Hydrogenated Styrene/Butadiene Copolymer, Hydrolyzed Candelilla Wax, Hydrolyzed Carnauba Wax, Hydrolyzed Chitosan, Hydrolyzed Gadidae Protein, Hydrolyzed Jojoba Esters, Hydrolyzed Sunflower Seed Wax, Hydrolyzed Wheat Protein, Hydrolyzed Wheat Protein/Cystine Bis-PG-Propyl Silanetriol Copolymer, Hydrolyzed Wheat Protein/Dimethicone PEG-7 Acetate, Hydrolyzed Wheat Protein/Dimethicone PEG-7 Phosphate Copolymer, Hydrolyzed Wheat Protein/PVP Crosspolymer, Hydroxybutyl Methylcellulose, Hydroxyethylcellulose, Hydroxyethyl Chitosan, Hydroxyethyl Ethylcellulose, Hydroxyethyl/Methoxyethyl Acrylate/Butyl Acrylate Copolymer, Hydroxyethyl/Methoxyethyl Acrylate Copolymer, Hydroxypropylcellulose, Hydroxypropyl Chitosan, Hydroxypropyl Guar, Hydroxypropyl Methylcellulose, Hydroxypropyl Methylcellulose Acetate/Succinate, Hydroxypropyl Oxidized Starch, Hydroxypropyltrimonium Hyaluronate, Hydroxypropyl Xanthan Gum, Isobutylene/Ethylmaleimide/Hydroxyethylmaleimide Copolymer, Isobutylene/MA Copolymer, Isobutylene/Sodium Maleate Copolymer, Isobutylmethacrylate/Bis-Hydroxypropyl Dimethicone Acrylate Copolymer, Isomerized Linoleic Acid, Isophorone Diamine/Cyclohexylamine/Isophthalic Acid/Azelaic Acid Copolymer, Isophoronediamine/isophthalic Acid/Pentaerythritol Copolymer, Isophorone Diamine/Isophthalic Acid/Trimethylolpropane Copolymer, Isopropyl Ester of PVM/MA Copolymer, 4,4′-Isopropylidenediphenol/Epichlorohydrin Copolymer, Lauryl Acrylate/VA Copolymer, Lauryl Methacrylate/Glycol Dimethacrylate Crosspolymer, Maltodextrin, Mannan, Melia Azadirachta Conditioned Media/Culture, Methacrylic Acid/Sodium Acrylamidomethyl Propane Sulfonate Copolymer, Methacryloyl Ethyl Betaine/Acrylates Copolymer, Methacryloyl Propyltrimethoxysilane, Methoxypolyoxymethylene Melamine, Methyl Ethylcellulose, Methyl Methacrylate/Acrylonitrile Copolymer, Methyl Methacrylate Crosspolymer, Methyl Methacrylate/Glycol Dimethacrylate Crosspolymer, Myrica Cerifera (Bayberry) Fruit Wax, Myroxylon Balsamum (Balsam Tolu) Resin, Myroxylon Pereirae (Balsam Peru) Resin, Nitrocellulose, Nylon-12/6/66 Copolymer, Octadecene/MA Copolymer, Octylacrylamide/Acrylates/Butylaminoethyl Methacrylate Copolymer, Oxymethylene/Melamine Copolymer, Palmitic Acid/Pentaerythritol/Stearic Acid/Terephthalic Acid Copolymer, PEG-150/Decyl Alcohol/SMDI Copolymer, PEG-7 Dimethicone, PEG/PPG-25/25 Dimethicone/Acrylates Copolymer, PEG-150/Stearyl Alcohol/SMDI Copolymer, Pentaerythritol/Terephthalic Acid Copolymer, Pentaerythrityl Cyclohexane Dicarboxylate, Perfluorononylethyl Stearyl Dimethicone, Phenylpropyldimethylsiloxysilicate, Phthalic Acid Denatured With Epoxy Resin Alkyd Resin, Phthalic Anhydride/Adipic Acid/Castor Oil/Neopentyl Glycol/PEG-3/Trimethylolpropane Copolymer, Phthalic Anhydride/Benzoic Acid/Glycerin Copolymer, Phthalic Anhydride/Benzoic Acid/Trimethylolpropane Copolymer, Phthalic Anhydride/Butyl Benzoic Acid/Propylene Glycol Copolymer, Phthalic Anhydride/Glycerin/Glycidyl Decanoate Copolymer, Phthalic Anhydride/Trimellitic Anhydride/Glycols Copolymer, Piperylene/Butene/Pentene Copolymer, Piperylene/Butene/Pentene/Pentadiene Copolymer, Pistacia Lentiscus (Mastic) Gum, Polianthes Tuberosa Extract, Polyacrylamide, Polyacrylamidomethylpropane Sulfonic Acid, Polyacrylate-1, Polyacrylate-2, Polyacrylate-5, Polyacrylate-6, Polyacrylic Acid, Polyamide-1, Polybeta-Alanine, Polybeta-Alanine/Glutaric Acid Crosspolymer, Polybutyl Acrylate, Polybutylene Terephthalate, Polychlorotrifluoroethylene, Polydiethyleneglycol Adipate/IPDI Copolymer, Polydimethylaminoethyl Methacrylate, Polyester-1, Polyester-2, Polyester-3, Polyethylacrylate, Polyethylene, Polyethylene Naphthalate, Polyethylene Terephthalate, Polyethylglutamate, Polyethylmethacrylate, Polyglucuronic Acid, Polyglyceryl-2 Diisostearate/IPDI Copolymer, Polyisobutene, Polylysine, Polymethacrylamide, Polymethacrylamidopropyltrimonium Methosulfate, Polymethacrylic Acid, Polymethyl Acrylate, Polymethylglutamate, Polymethyl Methacrylate, Polyoxyisobutylene/Methylene Urea Copolymer, Polyoxymethylene Melamine, Polypentaerythrityl Terephthalate, Polypentene, Polyperfluoroperhydrophenanthrene, Poly-p-Phenylene Terephthalamide, Polyphosphorylcholine Glycol Acrylate, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-43, Polyquaternium-44, Polyquaternium-45, Polyquaternium-46, Polyquaternium-47, Polyquaternium-48, Polyquaternium-49, Polyquaternium-50, Polyquaternium-51, Polyquaternium-56, Polyquaternium-57, Polyquaternium-61, Polysilicone-6, Polysilicone-8, Polysilicone-11, Polysilicone-14, Polystyrene, Polyurethane-1, Polyurethane-2, Polyurethane-4, Polyurethane-5, Polyurethane-6, Polyurethane-7, Polyurethane-8, Polyurethane-10, Polyurethane-11, Polyurethane-12, Polyurethane-13, Polyvinylacetal Diethylaminoacetate, Polyvinyl Acetate, Polyvinyl Alcohol, Polyvinyl Butyral, Polyvinylcaprolactam, Polyvinyl Chloride, Polyvinyl Imidazolinium Acetate, Polyvinyl Isobutyl Ether, Polyvinyl Laurate, Polyvinyl Methyl Ether, Polyvinyl Stearyl Ether, Potassium Acrylates/Acrylamide Copolymer, Potassium Acrylates/C10-30 Alkyl Acrylate Crosspolymer, Potassium Acrylates/Ethylhexyl Acrylate Copolymer, Potassium Butyl Ester of PVM/MA Copolymer, Potassium Carbomer, Potassium Carrageenan, Potassium Ethyl Ester of PVM/MA Copolymer, PPG-26/HDI Copolymer, PPG-17/IPDI/DMPA Copolymer, PPG-12/SMDI Copolymer, PPG-7/Succinic Acid Copolymer, PPG-26/TDI Copolymer, PPG-10 Tocophereth-30, PPG-20 Tocophereth-50, Propylene Glycol Diricinoleate/IPDI Copolymer, Pseudotsuga Menziesii (Balsam Oregon) Resin, Pullulan, PVM/MA Copolymer, PVM/MA Decadiene Crosspolymer, PVP, PVP Montmorillonite, PVP/VA/Itaconic Acid Copolymer, PVP/VA/Vinyl Propionate Copolymer, Quaternium-22, Rhizobian Gum, Rosin, Rubber Latex, Serum Albumin, Shellac, Sodium Acrylates/Acrolein Copolymer, Sodium Acrylates/Acrylonitrogens Copolymer, Sodium Acrylates/C10-30 Alkyl Acrylates Crosspolymer, Sodium Acrylates Copolymer, Sodium Acrylate/Vinyl Alcohol Copolymer, Sodium Butyl Ester of PVM/MA Copolymer, Sodium Carbomer, Sodium Carboxymethyl Chitin, Sodium Carboxymethyl Starch, Sodium Carrageenan, Sodium C₄-12 Olefin/Maleic Acid Copolymer, Sodium DVB/Acrylates Copolymer, Sodium Ethyl Ester of PVM/MA Copolymer, Sodium Isooctylene/MA Copolymer, Sodium MA/Diisobutylene Copolymer, Sodium MA/Vinyl Alcohol Copolymer, Sodium PG-Propyldimethicone Thiosulfate Copolymer, Sodium Polyacrylate, Sodium Polymethacrylate, Sodium Polystyrene Sulfonate, Sodium PVM/MA/Decadiene Crosspolymer, Sodium Styrene/Acrylates Copolymer, Sodium Tauride Acrylates/Acrylic Acid/Acrylonitrogens Copolymer, Starch/Acrylates/Acrylamide Copolymer, Starch Diethylaminoethyl Ether, Stearamidopropyl Dimethicone, Steareth-10 Allyl Ether/Acrylates Copolymer, Stearoyl Epoxy Resin, Stearyl HDI/PEG-50 Copolymer, Stearyl Methacrylate/Perfluorooctylethyl Methacrylate Copolymer, Stearylvinyl Ether/MA Copolymer, Styrax Benzoin Gum, Styrene/Acrylates/Acrylonitrile Copolymer, Styrene/Acrylates/Ammonium Methacrylate Copolymer, Styrene/Acrylates Copolymer, Styrene/Allyl Benzoate Copolymer, Styrene/DVB Crosspolymer, Styrene/Isoprene Copolymer, Styrene/MA Copolymer, Styrene/Methacrylamide/Acrylates Copolymer, Styrene/Methylstyrene/Indene Copolymer, Styrene/VA Copolymer, Styrene/VP Copolymer, Sucrose Benzoate/Sucrose Acetate Isobutyrate/Butyl Benzyl Phthalate Copolymer, Sucrose Benzoate/Sucrose Acetate Isobutyrate/Butyl Benzyl Phthalate/Methyl Methacrylate Copolymer, Sucrose Benzoate/Sucrose Acetate Isobutyrate Copolymer, TEA-Acrylates/Acrylonitrogens Copolymer, TEA-Diricinoleate, TEA-Diricinoleate/IPDI Copolymer, Terephthalic Acid/Isophthalic Acid/Sodium Isophthalic Acid Sulfonate/Glycol Copolymer, Tetradecyloctadecyl Behenate, Tetradecyloctadecyl Myristate, Tetradecyloctadecyl Stearate, Titanium Isostearates, Tosylamide/Epoxy Resin, Tosylamide/Formaldehyde Resin, Tricontanyl PVP, Triethylene Glycol Rosinate, Trimethylol Propane Cyclohexene Dicarboxylate, Trimethylolpropane Triacrylate, Trimethylpentanediol/Isophthalic Acid/Trimellitic Anhydride Copolymer, Trimethylsiloxysilicate/Dimethiconol Crosspolymer, Trimethylsiloxysilylcarbamoyl Pullulan, Triticum Vulgare (Wheat) Protein, Tromethamine Acrylates/Acrylonitrogens Copolymer, VA/Butyl Maleate/Isobornyl Acrylate Copolymer, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/Crotonic Acid/PEG-20M Copolymer, VA/DBM Copolymer, VA/Isobutyl Maleate/Vinyl Neodecanoate Copolymer, VA/Vinyl Butyl Benzoate/Crotonates Copolymer, VA/Vinyl Chloride Copolymer, Vinyl Acetate, Vinylamine/Vinyl Alcohol Copolymer, Vinyl Caprolactam/VP/Dimethylaminoethyl Methacrylate Copolymer, Vinyl Chloride/Vinyl Laurate Copolymer, VP/Dimethiconyl-acrylate/Polycarbamyl/Polyglycol Ester, VP/Dimethylaminoethylmethacrylate Copolymer, VP/Dimethylaminoethylmethacrylate/Polycarbamyl Polyglycol Ester, VP/Eicosene Copolymer, VP/Hexadecene Copolymer, VP/Polycarbamyl Polyglycol Ester, VP/VA Copolymer, Welan Gum, Yeast Beta-Glucan, Yeast Polysaccharides, Zein.

Polymers which fix hair in place, i.e. “setting” polymers assist in holding and/or building up the volume or fullness of the overall hairstyle. Film-forming polymers and gums are therefore generally typical substances for hair treatment agents such as hair setting preparations, hair mousses, hair waxes, hair sprays or leave-on conditioners. They are preferably used as such in the compositions according to the invention, if these are used in such agents. Substances which additionally lend the hair hydrophobic properties are preferred here, because they reduce the tendency of the hair to absorb moisture, i.e. water. This reduces the tendency of hair strands to hang down limply and thus ensures that a hairstyle retains its structure for a long time.

The “curl retention” test is often used as a test method in such cases. The use of at least one of these polymers in the agents according to the invention is therefore preferred according to the invention. Very particular preference is given to those polymers from this group which additionally also comprise setting properties. However, it is also preferred according to the invention for one setting and one film-forming polymer to be used in the agents according to the invention. It is most particularly preferred for the two polymers simultaneously to have both setting and film-forming properties, albeit optionally to different degrees.

Setting polymers assist in holding or building up the volume or fullness of the overall hairstyle. These “setting” polymers are simultaneously also film-forming polymers and therefore generally typical substances for hair treatment agents such as hair setting preparations, hair mousses, hair waxes, hairsprays. Film formation may in this respect take place only at points and connect only a few fibers together.

Because of the significance specifically of the setting polymers, these will be listed below explicitly by their INCI names. This list of polymers which are very particularly preferably to be used according to the invention obviously thus also precisely includes cationic polymers.

Examples of common film-forming, setting polymers are: Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates Copolymer, Acrylates/C₁₋₂ Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethyl-maleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate.

Preference is very particularly given to Acrylates/t-Butylacrylamide Copolymer, Octylacrylamide/Acrylates/Butylaminoethyl Methacrylate Copolymer, Polyurethane-1, Polyvinylcaprolactam and VP/VA Copolymer.

The film-forming and/or setting polymer is contained in the agent according to the invention preferably in a quantity of 0.01 to 40 wt. %, particularly preferably of 0.1 to 30 wt. %, very particularly preferably in a quantity of 0.1 to 10 wt. %. It goes without saying that a plurality of film-forming and/or setting polymers may also be contained in the agent according to the invention. In this respect, these film-forming and/or setting polymers may be both permanently and temporarily cationic, anionic, nonionic or amphoteric. Furthermore, the present invention also includes the recognition that, when at least two film-forming and/or setting polymers are used, they may of course have different charges. It may be preferable according to the invention for an ionic film-forming and/or setting polymer to be used together with an amphoteric and/or nonionic film-forming and/or setting polymer. The use of at least two oppositely charged film-forming and/or setting polymers is also preferred. In the latter case, a particular embodiment may in turn additionally contain at least one further amphoteric and/or nonionic film-forming and/or setting polymer.

Finally, the antistatic action of polymers is a further function essential to cosmetic agents. With the assistance of the electrical properties of these polymers, the electrical potential of the surfaces of the substrates treated with cosmetic agents (skin, nails and keratin fibers) is influenced. For example, in this way in hair care the effect known as the “fly-away effect” which is based on electrostatic repulsion of the hair fibers is reduced. However, skin feel on the skin surface is also influenced in this way. Some of these polymers here display their optimum action in a specific pH range. In the agents according to the invention the polymers preferred from this group of polymers are those which may simultaneously also be assigned to at least one of the groups of fixing and/or film-forming polymers. It goes without saying that the teaching according to the invention also includes the recognition that in the agents according to the invention in each case at least one antistatic, at least one fixing and at least one film-forming polymer may also be used. It is preferable, however, to select the polymers such that at least one of the polymers comprises at least two of the desired properties. It is most particularly preferred according to the invention for the polymer also to exhibit a further property in addition to the three very particularly important properties setting, fixing and film formation.

Examples of such antistatic polymers are:

Acrylamidopropyltrimonium Chloride/Acrylamide Copolymer, Acrylamidopropyltrimonium Chloride/Acrylates Copolymer, AMP-Isostearoyl Gelatin/Keratin Amino Acids/Lysine Hydroxypropyltrimonium Chloride, Benzyltrimonium Hydrolyzed Collagen, Caesalpinia Spinosa Hydroxypropyltrimonium Chloride, Cocamidopropyldimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Dimethicone Hydroxypropyl Trimonium Chloride, Dimethicone Propylethylenediamine Behenate, Dimethicone Propyl PG-Betaine, Ditallow Dimonium Cellulose Sulfate, Gelatin/Keratin Amino Acids/Lysine Hydroxypropyltrimonium Chloride, Gelatin/Lysine/Polyacrylamide Hydroxypropyltrimonium Chloride, Beta-Glucan Hydroxypropyltrimonium Chloride, Guar Hydroxypropyltrimonium Chloride, Hydrogenated Starch Hydrolysate Hydroxypropyltrimonium Chloride, Hydroxypropyl Guar Hydroxypropyltrimonium Chloride, Hydroxypropyltrimonium Gelatin, Hydroxypropyltrimonium Honey, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Jojoba Protein, Hydroxypropyltrimonium Hydrolyzed Keratin, Hydroxypropyltrimonium Hydrolyzed Rice Bran Protein, Hydroxypropyltrimonium Hydrolyzed Silk, Hydroxypropyltrimonium Hydrolyzed Soy Protein, Hydroxypropyltrimonium Hydrolyzed Vegetable Protein, Hydroxypropyltrimonium Hydrolyzed Wheat Protein, Hydroxypropyltrimonium Hydrolyzed Wheat Protein/Siloxysilicate, Hydroxypropyltrimonium Hydrolyzed Wheat Starch, Hydroxypropyltrimonium Hydrolyzed Whey, Laurdimonium Hydroxypropyl Hydrolyzed Jojoba Protein, Laurdimonium Hydroxypropyl Hydrolyzed Wheat Protein, Laurdimonium Hydroxypropyl Hydrolyzed Wheat Protein/Siloxysilicate, Laurdimonium Hydroxypropyl Hydrolyzed Wheat Starch, Laurdimonium Hydroxypropyl Wheat Amino Acids, Laur/Myrist/Palmitamidobutyl Guanidine Acetate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Oleamidopropyl Dimethylamine Hydrolyzed Collagen, Oleamidopropyldimonium Hydroxypropyl Hydrolyzed Collagen, PEG-2 Coco-Benzonium Chloride, PEG-10 Coco-Benzonium Chloride, PEG-2 Cocomonium Chloride, PEG-15 Cocomonium Chloride, PEG-5 Cocomonium Methosulfate, PEG-15 Cocomonium Methosulfate, PEG-15 Cocopolyamine, PEG-9 Diethylmonium Chloride, PEG-25 Diethylmonium Chloride, PEG-2 Dimeadowfoamamidoethylmonium Methosulfate, PEG-3 Dioleoylamidoethylmonium Methosulfate, PEG-3 Distearoylamidoethylmonium Methosulfate, PEG-4 Distearylethonium Ethosulfate, PEG-2 Hydrogenated Tallow Amine, PEG-5 Hydrogenated Tallow Amine, PEG-8 Hydrogenated Tallow Amine, PEG-10 Hydrogenated Tallow Amine, PEG-15 Hydrogenated Tallow Amine, PEG-20 Hydrogenated Tallow Amine, PEG-30 Hydrogenated Tallow Amine, PEG-40 Hydrogenated Tallow Amine, PEG-50 Hydrogenated Tallow Amine, PEG-15 Hydrogenated Tallowmonium Chloride, PEG-5 Isodecyloxypropylamine, PEG-2 Lauramine, PEG-5 Oleamine, PEG-15 Oleamine, PEG-30 Oleamine, PEG-2 Oleammonium Chloride, PEG-15 Oleammonium Chloride, PEG-12 Palmitamine, PEG-8 Palmitoyl Methyl Diethonium Methosulfate, PEG/PPG-1/25 Diethylmonium Chloride, PEG-2 Rapeseedamine, PEG-2 Soyamine, PEG-5 Soyamine, PEG-8 Soyamine, PEG-10 Soyamine, PEG-15 Soyamine, PEG-2 Stearamine, PEG-5 Stearamine, PEG-10 Stearamine, PEG-15 Stearamine, PEG-50 Stearamine, PEG-2 Stearmonium Chloride, PEG-15 Stearmonium Chloride, PEG-5 Stearyl Ammonium Chloride, PEG-5 Stearyl Ammonium Lactate, PEG-10 Stearyl Benzonium Chloride, PEG-6 Stearylguanidine, PEG-5 Tallow Amide, PEG-2 Tallow Amine, PEG-7 Tallow Amine, PEG-11 Tallow Amine, PEG-15 Tallow Amine, PEG-20 Tallow Amine, PEG-25 Tallow Amine, PEG-3 Tallow Aminopropylamine, PEG-10 Tallow Aminopropylamine, PEG-15 Tallow Aminopropylamine, PEG-20 Tallow Ammonium Ethosulfate, PEG-5 Tallow Benzonium Chloride, PEG-15 Tallow Polyamine, PEG-3 Tallow Propylenedimonium Dimethosulfate, PG-Hydroxyethylcellulose Cocodimonium Chloride, PG-Hydroxyethylcellulose Lauryldimonium Chloride, PG-Hydroxyethylcellulose Stearyldimonium Chloride, Polymethacrylamidopropyltrimonium Chloride, Polymethacrylamidopropyltrimonium Methosulfate, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-43, Polyquaternium-44, Polyquaternium-45, Polyquaternium-46, Polyquaternium-48, Polyquaternium-49, Polyquaternium-50, Polyquaternium-54, Polyquaternium-60, Polysilicone-1, Polyvinyl Imidazolinium Acetate, PPG-2 Cocamine, PPG-9 Diethylmonium Chloride, PPG-25 Diethylmonium Chloride, PPG-40 Diethylmonium Chloride, PPG-2 Hydrogenated Tallowamine, PPG-24-PEG-21 Tallowaminopropylamine, PPG-2 Tallowamine, PPG-3 Tallow Aminopropylamine, Propyltrimonium Hydrolyzed Collagen, Propyltrimonium Hydrolyzed Soy Protein, Propyltrimonium Hydrolyzed Wheat Protein, Quaternium-8, Quaternium-14, Quaternium-15, Quaternium-16, Quaternium-18, Quaternium-18 Methosulfate, Quaternium-22, Quaternium-24, Quaternium-26, Quaternium-27, Quaternium-30, Quaternium-33, Quaternium-43, Quaternium-45, Quaternium-51, Quaternium-52, Quaternium-53, Quaternium-56, Quaternium-60, Quaternium-61, Quaternium-63, Quaternium-70, Quaternium-71, Quaternium-72, Quaternium-73, Quaternium-75, Quaternium-76 Hydrolyzed Collagen, Quaternium-77, Quaternium-78, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, Quaternium-79 Hydrolyzed Wheat Protein, Quaternium-80, Quaternium-81, Quaternium-82, Quaternium-83, Quaternium-86, Quaternium-88, Quaternium-89, Quaternium-90, Silicone Quaternium-2 Panthenol Succinate, Steardimonium Hydroxypropyl Hydrolyzed Casein, Steardimonium Hydroxypropyl Hydrolyzed Collagen, Steardimonium Hydroxypropyl Hydrolyzed Jojoba Protein, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Triethonium Hydrolyzed Collagen Ethosulfate, Trigonella Foenum-Graecum Hydroxypropyltrimonium Chloride, Wheat Germamidopropyldimonium Hydroxypropyl Hydrolyzed Wheat Protein, Wheat Germamidopropyl Epoxypropyldimonium Chloride, Wheatgermamidopropyl Ethyldimonium Ethosulfate.

It goes without saying that emulsion-stabilizing polymers also belong to the polymers preferred according to the invention. These include polymers which significantly assist in building and stabilizing emulsions (O/W and W/O and multiple emulsions). Surfactants and emulsifiers are of course the essential constituents, but stabilizing polymers contribute to a reduction in the coalescence of the emulsified droplets by having a positive influence on the continuous or disperse phase. This positive influence may be based on electrical repulsion, an increase in viscosity or film formation on the droplet surface. These properties of the relevant polymers may also particularly advantageously be used in the compositions according to the invention to dissolve the pulverulent compositions according to the invention in water before and/or during application of the powder.

Examples of such polymers are Acrylamide/Sodium Acryloyldimethyltaurate Copolymer, Acrylates/Aminoacrylates/C10-30 Alkyl PEG-20 Itaconate Copolymer, Acrylates/C10-30 Alkyl Acrylate Crosspolymer, Acrylates/Stearyl Methacrylate Copolymer, Acrylates/Vinyl Isodecanoate Crosspolymer, Alcaligenes Polysaccharides, Allyl Methacrylates Crosspolymer, Ammonium Acryloyldimethyltaurate/Beheneth-25 Methacrylate Crosspolymer, Ammonium Acryloyldimethyltaurate/Vinyl Formamide Copolymer, Ammonium Alginate, Ammonium Phosphatidyl Rapeseedate, Ammonium Polyacrylate, Ammonium Polyacryloyldimethyl Taurate, Ammonium Shellacate, Arachidyl Alcohol, Astragalus Gummifer Gum, Beeswax, Bentonite, Calcium Carboxymethyl Cellulose, Calcium Carrageenan, Calcium Potassium Carbomer, Calcium Starch Octenylsuccinate, C₁₋₅ Alkyl Galactomannan, C18-38 Alkyl Hydroxystearoyl Stearate, Carbomer, Carboxymethyl Hydroxyethylcellulose, Carboxymethyl Hydroxypropyl Guar, Cellulose Acetate Propionate Carboxylate, Cellulose Gum, Ceratonia Siliqua Gum, Cetyl Hydroxyethylcellulose, Chitosan Lauroyl Glycinate, Cholesterol, Cholesterol/HDI/Pullulan Copolymer, Corn Starch/Acrylamide/Sodium Acrylate Copolymer, C12-14 Sec-Pareth-3, C12-14 Sec-Pareth-5, C12-14 Sec-Pareth-7, C12-14 Sec-Pareth-8, C12-14 Sec-Pareth-9, C12-14 Sec-Pareth-12, C12-14 Sec-Pareth-15, C12-14 Sec-Pareth-20, C12-14 Sec-Pareth-30, C12-14 Sec-Pareth-40, C12-14 Sec-Pareth-50, Cyamopsis Tetragonoloba (Guar) Gum, Dimethicone Crosspolymer, Dimethicone Crosspolymer-2, Dimethicone Ethoxy Glucoside, Euphorbia Cerifera (Candelilla) Wax, Gellan Gum, Hydrolyzed Beeswax, Hydrolyzed Candelilla Wax, Hydrolyzed Carnauba Wax, Hydrolyzed Collagen PG-Propyl Dimethiconol, Hydrolyzed Sunflower Seed Wax, Hydroxybutyl Methylcellulose, Hydroxyethyl Acrylate/Sodium Acryloyldimethyl Taurate Copolymer, Hydroxyethylcellulose, Hydroxyethyl Ethylcellulose, Hydroxyethyl Isostearyloxy Isopropanolamine, Hydroxypropylcellulose, Hydroxypropyl Cyclodextrin, Hydroxypropyl Guar, Hydroxypropyl Methylcellulose, Hydroxypropyl Xanthan Gum, Isopropyl Ester of PVM/MA Copolymer, Lanolin, Lanolin Alcohol, Magnesium Alginate, Maltodextrin, Methoxy PEG-17/Dodecyl Glycol Copolymer, Methoxy PEG-22/Dodecyl Glycol Copolymer, Methylcellulose, Methyl Hydroxyethylcellulose, Microcrystalline Cellulose, Microcrystalline Wax, Montmorillonite, Moroccan Lava Clay, Myrica Cerifera (Bayberry) Fruit Wax, Octadecene/MA Copolymer, Oleic/Linoleic/Linolenic Polyglycerides, Ozokerite, Pectin, PEG-350, PEG-400, PEG-500, PEG-12 Carnauba, PEG-12 Dimethicone Crosspolymer, PEG-22/Dodecyl Glycol Copolymer, PEG-45/Dodecyl Glycol Copolymer, PEG-6 Hydrogenated Palmamide, PEG-100/IPDI Copolymer, PEG-2M, PEG-5M, PEG-7M, PEG-9M, PEG-14M, PEG-20M, PEG-23M, PEG-25M, PEG-45M, PEG-65M, PEG-90M, PEG-115M, PEG-160M, PEG/PPG-20/23 Dimethicone, PEG/PPG-23/6 Dimethicone, PEG/PPG-8/3 Laurate, PEG/PPG-10/3 Oleyl Ether Dimethicone, Polyacrylic Acid, Polyethylene, Polyethylene/Isopropyl Maleate/MA Copolyol, Polyglyceryl-2 Diisostearate/IPDI Copolymer, Polypropylene Terephthalate, Polysilicone-16, Polyvinyl Acetate, Potassium Alginate, Potassium Carbomer, Potassium Carrageenan, Potassium Dextrin Octenylsuccinate, Potassium Polyacrylate, Potassium Undecylenoyl Alginate, Potassium Undecylenoyl Carrageenan, Potassium Undecylenoyl Hydrolyzed Corn Protein, Potassium Undecylenoyl Hydrolyzed Soy Protein, Potassium Undecylenoyl Hydrolyzed Wheat Protein, PPG-3 C12-14 Sec-Pareth-7, PPG-4 C12-14 Sec-Pareth-5, PPG-5 C12-14 Sec-Pareth-7, PPG-5 C12-14 Sec-Pareth-9, PPG-2 Tocophereth-5, PPG-5 Tocophereth-2, PPG-10 Tocophereth-30, PPG-20 Tocophereth-50, PVM/MA Copolymer, PVP, PVP/Decene Copolymer, PVP Montmorillonite, Pyrus Malus (Apple) Fiber, Saccharated Lime, Sclerotium Gum, Sodium Acrylate/Acryloyldimethyl Taurate Copolymer, Sodium Acrylates/Vinyl Isodecanoate Crosspolymer, Sodium Acrylate/Vinyl Alcohol Copolymer, Sodium Carbomer, Sodium Carboxymethyl Dextran, Sodium Carboxymethyl Starch, Sodium Carrageenan, Sodium Cellulose Sulfate, Sodium C4-12 Olefin/Maleic Acid Copolymer, Sodium Cyclodextrin Sulfate, Sodium Dextrin Octenylsuccinate, Sodium Polyacrylate, Sodium Polyacrylate Starch, Sodium Polyacryloyidimethyl Taurate, Sodium Polymethacrylate, Sodium Polynaphthalenesulfonate, Sodium Polystyrene Sulfonate, Sodium Starch Octenylsuccinate, Sodium/TEA-Undecylenoyl Alginate, Sodium/TEA-Undecylenoyl Carrageenan, Sodium Tocopheryl Phosphate, Starch Hydroxypropyltrimonium Chloride, Stearylvinyl Ether/MA Copolymer, Sterculia Urens Gum, Styrene/MA Copolymer, Sucrose Polypalmate, Synthetic Beeswax, Synthetic Wax, Tamarindus Indica Seed Gum, TEA-Alginate, TEA-Dextrin Octenylsuccinate, Undecylenoyl Inulin, Undecylenoyl Xanthan Gum, Welan Gum, Xanthan Gum, Zinc Undecylenoyl Hydrolyzed Wheat Protein.

Polymers may increase the viscosity of aqueous and non-aqueous phases in cosmetic preparations. In aqueous phases, their viscosity-increasing function is based on their solubility in water or their hydrophilic nature. They are applied both in surfactant systems and in systems in the form of emulsions. This property of the polymers is also advantageous in the powders according to the invention before and/or during application.

Some examples of typical polymeric thickeners for aqueous systems are listed below:

Acrylamides Copolymer, Acrylamide/Sodium Acrylate Copolymer, Acrylamide/Sodium Acryloyidimethyltaurate Copolymer, Acrylates/Acetoacetoxyethyl Methacrylate Copolymer, Acrylates/Beheneth-25 Methacrylate Copolymer, Acrylates/C10-30 Alkyl Acrylate Crosspolymer, Acrylates/Ceteth-20 Itaconate Copolymer, Acrylates/Ceteth-20 Methacrylate Copolymer, Acrylates/Laureth-25 Methacrylate Copolymer, Acrylates/Palmeth-25 Acrylate Copolymer, Acrylates/Palmeth-25 Itaconate Copolymer, Acrylates/Steareth-50 Acrylate Copolymer, Acrylates/Steareth-20 Itaconate Copolymer, Acrylates/Steareth-20 Methacrylate Copolymer, Acrylates/Stearyl Methacrylate Copolymer, Acrylates/Vinyl Isodecanoate Crosspolymer, Acrylic Acid/Acrylonitrogens Copolymer, Agar, Agarose, Alcaligenes Polysaccharides, Algin, Alginic Acid, Ammonium Acrylates/Acrylonitrogens Copolymer, Ammonium Acrylates Copolymer, Ammonium Acryloyldimethyltaurate/Vinyl Formamide Copolymer, Ammonium Acryloyldimethyltaurate/VP Copolymer, Ammonium Alginate, Ammonium Polyacryloyldimethyl Taurate, Amylopectin, Ascorbyl Methylsilanol Pectinate, Astragalus Gummifer Gum, Attapulgite, Avena Sativa (Oat) Kernel Flour, Bentonite, Butoxy Chitosan, Caesalpinia Spinosa Gum, Calcium Alginate, Calcium Carboxymethyl Cellulose, Calcium Carrageenan, Calcium Potassium Carbomer, Calcium Starch Octenylsuccinate, C20-40 Alkyl Stearate, Carbomer, Carboxybutyl Chitosan, Carboxymethyl Chitin, Carboxymethyl Chitosan, Carboxymethyl Dextran, Carboxymethyl Hydroxyethylcellulose, Carboxymethyl Hydroxypropyl Guar, Cellulose Acetate Propionate Carboxylate, Cellulose Gum, Ceratonia Siliqua Gum, Cetyl Hydroxyethylcellulose, Cholesterol/HDI/Pullulan Copolymer, Cholesteryl Hexyl Dicarbamate Pullulan, Cyamopsis Tetragonoloba (Guar) Gum, Diglycol/CHDM/Isophthalates/SIP Copolymer, Dihydrogenated Tallow Benzylmonium Hectorite, Dimethicone Crosspolymer-2, Dimethicone Propyl PG-Betaine, DMAPA Acrylates/Acrylic Acid/Acrylonitrogens Copolymer, Ethylene/Sodium Acrylate Copolymer, Gelatin, Gellan Gum, Glyceryl Alginate, Glycine Soja (Soybean) Flour, Guar Hydroxypropyltrimonium Chloride, Hectorite, Hydrated Silica, Hydrogenated Potato Starch, Hydroxybutyl Methylcellulose, Hydroxyethyl Acrylate/Sodium Acryloyldimethyl Taurate Copolymer, Hydroxyethylcellulose, Hydroxyethyl Chitosan, Hydroxyethyl Ethylcellulose, Hydroxypropylcellulose, Hydroxypropyl Chitosan, Hydroxypropyl Ethylenediamine Carbomer, Hydroxypropyl Guar, Hydroxypropyl Methylcellulose, Hydroxypropyl Methylcellulose Stearoxy Ether, Hydroxypropyl Starch, Hydroxypropyl Starch Phosphate, Hydroxypropyl Xanthan Gum, Hydroxystearamide MEA, Isobutylene/Sodium Maleate Copolymer, Lithium Magnesium Silicate, Lithium Magnesium Sodium Silicate, Macrocystis Pyrifera (Kelp), Magnesium Alginate, Magnesium Aluminum Silicate, Magnesium Silicate, Magnesium Trisilicate, Methoxy PEG-22/Dodecyl Glycol Copolymer, Methylcellulose, Methyl Ethylcellulose, Methyl Hydroxyethylcellulose, Microcrystalline Cellulose, Montmorillonite, Moroccan Lava Clay, Natto Gum, Nonoxynyl Hydroxyethylcellulose, Octadecene/MA Copolymer, Pectin, PEG-800, PEG-Crosspolymer, PEG-150/Decyl Alcohol/SMDI Copolymer, PEG-175 Diisostearate, PEG-190 Distearate, PEG-15 Glyceryl Tristearate, PEG-140 Glyceryl Tristearate, PEG-240/HDI Copolymer Bis-Decyltetradeceth-20 Ether, PEG-100/IPDI Copolymer, PEG-180/Laureth-50/TMMG Copolymer, PEG-10/Lauryl Dimethicone Crosspolymer, PEG-15/Lauryl Dimethicone Crosspolymer, PEG-2M, PEG-5M, PEG-7M, PEG-9M, PEG-14M, PEG-20M, PEG-23M, PEG-25M, PEG-45M, PEG-65M, PEG-90M, PEG-115M, PEG-160M, PEG-120 Methyl Glucose Trioleate, PEG-180/Octoxynol-40/TMMG Copolymer, PEG-150 Pentaerythrityl Tetrastearate, PEG-4 Rapeseedamide, PEG-150/Stearyl Alcohol/SMDI Copolymer, Polyacrylate-3, Polyacrylic Acid, Polycyclopentadiene, Polyether-1, Polyethylene/Isopropyl Maleate/MA Copolyol, Polymethacrylic Acid, Polyquaternium-52, Polyvinyl Alcohol, Potassium Alginate, Potassium Aluminum Polyacrylate, Potassium Carbomer, Potassium Carrageenan, Potassium Polyacrylate, Potato Starch Modified, PPG-14 Laureth-60 Hexyl Dicarbamate, PPG-14 Laureth-60 Isophoryl Dicarbamate, PPG-14 Palmeth-60 Hexyl Dicarbamate, Propylene Glycol Alginate, PVP/Decene Copolymer, PVP Montmorillonite, Rhizobian Gum, Ricinoleic Acid/Adipic Acid/AEEA Copolymer, Sclerotium Gum, Sodium Acrylate/Acryloyldimethyl Taurate Copolymer, Sodium Acrylates/Acrolein Copolymer, Sodium Acrylates/Acrylonitrogens Copolymer, Sodium Acrylates Copolymer, Sodium Acrylates/Vinyl Isodecanoate Crosspolymer, Sodium Acrylate/Vinyl Alcohol Copolymer, Sodium Carbomer, Sodium Carboxymethyl Chitin, Sodium Carboxymethyl Dextran, Sodium Carboxymethyl Beta-Glucan, Sodium Carboxymethyl Starch, Sodium Carrageenan, Sodium Cellulose Sulfate, Sodium Cyclodextrin Sulfate, Sodium Hydroxypropyl Starch Phosphate, Sodium Isooctylene/MA Copolymer, Sodium Magnesium Fluorosilicate, Sodium Polyacrylate, Sodium Polyacrylate Starch, Sodium Polyacryloyidimethyl Taurate, Sodium Polymethacrylate, Sodium Polystyrene Sulfonate, Sodium Silicoaluminate, Sodium Starch Octenylsuccinate, Sodium Stearoxy PG-Hydroxyethylcellulose Sulfonate, Sodium Styrene/Acrylates Copolymer, Sodium Tauride Acrylates/Acrylic Acid/Acrylonitrogens Copolymer, Solanum Tuberosum (Potato) Starch, Starch/Acrylates/Acrylamide Copolymer, Starch Hydroxypropyltrimonium Chloride, Steareth-60 Cetyl Ether, Steareth-100/PEG-136/HDI Copolymer, Sterculia Urens Gum, Synthetic Fluorphlogopite, Tamarindus Indica Seed Gum, Tapioca Starch, TEA-Alginate, TEA-Carbomer, Triticum Vulgare (Wheat) Starch, Tromethamine Acrylates/Acrylonitrogens Copolymer, Tromethamine Magnesium Aluminum Silicate, Welan Gum, Xanthan Gum, Yeast Beta-Glucan, Yeast Polysaccharides, Zea Mays (Corn) Starch.

A further possible way of increasing the viscosity of cosmetics is to thicken the non-aqueous phase, the lipid phase of the cosmetic agent. Polymers are used for this purpose which are water-insoluble but are compatible with lipids. They are also used for gelation of cosmetics with elevated lipid contents. This likewise makes a substantial contribution to the excellent application of the powder according to the invention. With these polymers, the viscosity of the composition forming on dissolution is excellently controlled.

Some of these polymers are listed below:

Acrylates/C10-30 Alkyl Acrylate Crosspolymer, Adipic Acid/PPG-10 Copolymer, Allyl Methacrylates Crosspolymer, Alumina Magnesium Metasilicate, Aluminum Starch Octenylsuccinate, Beeswax, Behenyl Methacrylate/Perfluorooctylethyl Methacrylate Copolymer, Bispolyethylene Dimethicone, Butadiene/Acrylonitrile Copolymer, Butylene/Ethylene Copolymer, Butylene/Ethylene/Styrene Copolymer, Butylene Glycol Montanate, Butyrospermum Parkii (Shea Butter), C29-70 Acid, C23-43 Acid Pentaerythritol Tetraester, C20-24 Alkyl Dimethicone, C24-28 Alkyl Dimethicone, C1-5 Alkyl Galactomannan, C18-38 Alkyl Hydroxystearoyl Stearate, C20-24 Alkyl Methicone, C24-28 Alkyl Methicone, C30-45 Alkyl Methicone, Candelilla Wax Hydrocarbons, C10-30 Cholesterol/Lanosterol Esters, Cellobiose Octanonanoate, Ceresin, Cerotic Acid, Cetearyl Dimethicone/Vinyl Dimethicone Crosspolymer, Chlorinated Paraffin, Cholesterol, Cholesteryl Acetate, Cholesteryl Hydroxystearate, Cholesteryl Isostearate, Cholesteryl Macadamiate, Cholesteryl Stearate, C10-40 Hydroxyalkyl Acid Cholesterol Esters, C10-40 Isoalkyl Acid Cholesterol Esters, C10-40 Isoalkyl Acid Octyldodecanol Esters, C10-40 Isoalkyl Acid Phytosterol Esters, C10-40 Isoalkyl Acid Triglyceride, C30-38 Olefin/Isopropyl Maleate/MA Copolymer, Copal, Corn Starch Modified, C₆₋₁₄ Perfluoroalkylethyl Acrylate/HEMA Copolymer, C₆₋₁₄ Polyolefin, Decene/Butene Copolymer, Dihydrogenated Tallow Benzylmonium Hectorite, Dilinoleic Acid/Ethylenediamine Copolymer, Dilinoleic Acid/Sebacic Acid/piperazine/Ethylenediamine Copolymer, Dimethicone Crosspolymer, Dimethicone/Phenyl Vinyl Dimethicone Crosspolymer, Dimethicone/Vinyl Dimethicone Crosspolymer, Dimethicone/Vinyltrimethylsiloxysilicate Crosspolymer, Diphenyl Dimethicone/Vinyl Diphenyl Dimethicone/Silsesquioxane Crosspolymer, Divinyidimethicone/Dimethicone Crosspolymer, Dodecanedioic Acid/Cetearyl Alcohol/Glycol Copolymer, Ethylcellulose, Ethylene/Acrylic Acid Copolymer, Ethylene/Acrylic Acid/VA Copolymer, Ethylenediamine/Dimer Tallate Copolymer Bis-Hydrogenated Tallow Amide, Ethylenediamine/Stearyl Dimer Dilinoleate Copolymer, Ethylenediamine/Stearyl Dimer Tallate Copolymer, Ethylene/Octene Copolymer, Ethylene/Propylene Copolymer, Ethylene/Propylene/Styrene Copolymer, Euphorbia Cerifera (Candelilla) Wax, Hydrogenated Butylene/Ethylene/Styrene Copolymer, Hydrogenated Ethylene/Propylene/Styrene Copolymer, Hydrogenated Japan Wax, Hydrogenated Polyisobutene, Hydrogenated Styrene/Butadiene Copolymer, Hydrogenated Styrene/Methyl Styrene/Indene Copolymer, Hydroxypropylcellulose, Isobutylene/Isoprene Copolymer, Lithium Oxidized Polyethylene, Methoxy PEG-17/Dodecyl Glycol Copolymer, Methoxy PEG-22/Dodecyl Glycol Copolymer, Methyl Methacrylate Crosspolymer, Methylstyrene/Vinyltoluene Copolymer, Microcrystalline Wax, Montan Acid Wax, Montan Wax, Myrica Cerifera (Bayberry) Fruit Wax, Nylon-611/Dimethicone Copolymer, Octadecene/MA Copolymer, Oleic/Linoleic/Linolenic Polyglycerides, Ouricury Wax, Oxidized Beeswax, Oxidized Microcrystalline Wax, Oxidized Polyethylene, Oxidized Polypropylene, Ozokerite, Paraffin, PEG-18 Castor Oil Dioleate, PEG-10 Dimethicone Crosspolymer, PEG-12 Dimethicone Crosspolymer, PEG-5 Hydrogenated Castor Oil Isostearate, PEG-10 Hydrogenated Castor Oil Isostearate, PEG-20 Hydrogenated Castor Oil Isostearate, PEG-30 Hydrogenated Castor Oil Isostearate, PEG-40 Hydrogenated Castor Oil Isostearate, PEG-50 Hydrogenated Castor Oil Isostearate, PEG-58 Hydrogenated Castor Oil Isostearate, PEG-50 Hydrogenated Castor Oil Succinate, PEG-5 Hydrogenated Castor Oil Triisostearate, PEG-10 Hydrogenated Castor Oil Triisostearate, PEG-15 Hydrogenated Castor Oil Triisostearate, PEG-20 Hydrogenated Castor Oil Triisostearate, PEG-30 Hydrogenated Castor Oil Triisostearate, PEG-40 Hydrogenated Castor Oil Triisostearate, PEG-60 Hydrogenated Castor Oil Triisostearate, PEG-5 Lanolinamide, PEG-5 Oleamide Dioleate, Phthalic Anhydride/Butyl Benzoic Acid/Propylene Glycol Copolymer, Phthalic Anhydride/Glycerin/Glycidyl Decanoate Copolymer, Phthalic Anhydride/Trimellitic Anhydride/Glycols Copolymer, Piperylene/Butene/Pentene Copolymer, Polybutene, Polybutylene Terephthalate, Polycyclopentadiene, Polydipentene, Polyethylene, Polyethylene Terephthalate, Polyglyceryl-3 Polyricinoleate, Polyglyceryl-4 Polyricinoleate, Polyglyceryl-5 Polyricinoleate, Polyglyceryl-10 Polyricinoleate, Polyisobutene, Polyisoprene, Polypentene, Polyperfluoroethoxymethoxy Difluoromethyl Distearamide, Polypropylene, Polysilicone-4, Polysilicone-5, Polysilicone-17, Polystyrene, Polyvinyl Butyral, Polyvinyl Laurate, Potassium Oxidized Microcrystalline Wax, Potassium PEG-50 Hydrogenated Castor Oil Succinate, PVM/MA Decadiene Crosspolymer, PVP/Decene Copolymer, Rhus Succedanea Fruit Wax, Rosin, Silica Dimethicone Silylate, Silica Dimethyl Silylate, Simmondsia Chinensis (Jojoba) Seed Wax, Sodium PVM/MA/Decadiene Crosspolymer, Spent Grain Wax, Steareth-10 Allyl Ether/Acrylates Copolymer, Steareth-60 Cetyl Ether, Stearoxymethicone/Dimethicone Copolymer, Stearyl Methacrylate/Perfluorooctylethyl Methacrylate Copolymer, Styrene/Methacrylamide/Acrylates Copolymer, Synthetic Beeswax, Synthetic Candelilla Wax, Synthetic Carnauba, Synthetic Japan Wax, Synthetic Wax, TDI Oxidized Microcrystalline Wax, Tricontanyl PVP, Trifluoropropyl Dimethicone Crosspolymer, Trifluoropropyl Dimethicone/Trifluoropropyl Divinyldimethicone Crosspolymer, Trifluoropropyl Dimethicone/Vinyl Trifluoropropyl Dimethicone/Silsesquioxane Crosspolymer, Trimethylpentanediol/Isophthalic Acid/Trimellitic Anhydride Copolymer, Trimethylsiloxysilicate/Dimethiconol Crosspolymer, Vinyl Dimethicone/Lauryl Dimethicone Crosspolymer, Vinyl Dimethicone/Methicone Silsesquioxane Crosspolymer, VP/Eicosene Copolymer, VP/Hexadecene Copolymer.

It goes without saying that filled or unfilled microparticles may be used in the composition according to the invention, both to achieve specific effects, such as the release of an active ingredient from the capsules, or to achieve particular visual, esthetic effects of the overall formulation. In this case it may be particularly advantageous for polymers to be incorporated as suspending agents. Suspending agents facilitate dispersion of solids in liquids. In this case, the polymers coat the surface of the solid particles by adsorption and thereby change the surface properties of the solids. Examples of these polymers are listed below:

Acrylates Copolymer, Acrylates/Methoxy PEG-15 Methacrylate Copolymer, Acrylates/Vinyl Isodecanoate Crosspolymer, Acrylates/VP Copolymer, Acrylic Acid/Acrylamidomethyl Propane Sulfonic Acid Copolymer, Ammonium Styrene/Acrylates Copolymer, Ammonium VA/Acrylates Copolymer, Bentonite, Biotite, Calcium Lignosulfonate, Corn Starch/Acrylamide/Sodium Acrylate Copolymer, C6-14 Perfluoroalkylethyl Acrylate/HEMA Copolymer, Diallyloxyneohexyl Zirconium Tridecanoate, Dihydrogenated Tallow Benzylmonium Hectorite, Dimethicone Crosspolymer, Dimethiconol/Stearyl Methicone/Phenyl Trimethicone Copolymer, Dimethylol Urea/Phenol/Sodium Phenolsulfonate Copolymer, Disodium Methylene Dinaphthalenesulfonate, Disteardimonium Hectorite, Ethylene/MA Copolymer, Ethylene/VA Copolymer, Ethylhexyl Hydroxystearoyl Hydroxystearate, Hectorite, Hydroxyethyl Acrylate/Sodium Acryloyldimethyl Taurate Copolymer, Hydroxyethyl PEI-1000, Hydroxyethyl PEI-1500, Hydroxypropyl Starch, Hydroxypropyltrimonium Maltodextrin Crosspolymer, Isobutylene/MA Copolymer, Isopropyl Ester of PVM/MA Copolymer, Maltodextrin, Methacryloyl Ethyl Betaine/Acrylates Copolymer, Methoxy PEG-17/Dodecyl Glycol Copolymer, Methoxy PEG-22/Dodecyl Glycol Copolymer, Myristoyl/PCA Chitin, Nitrocellulose, PEG-18 Castor Oil Dioleate, PEG-150/Decyl Alcohol/SMDI Copolymer, PEG-12 Dimethicone Crosspolymer, PEG-150/Stearyl Alcohol/SMDI Copolymer, PEI-7, PEI-10, PEI-15, PEI-30, PEI-35, PEI-45, PEI-250, PEI-275, PEI-700, PEI-1000, PEI-1400, PEI-1500, PEI-1750, PEI-2500, PEI-14M, Perfluorononyl Octyldodecyl Glycol Meadowfoamate, Perlite, Phosphonobutanetricarboxylic Acid, Polyacrylamidomethylpropane Sulfonic Acid, Polycaprolactone, Polyethylacrylate, Polyhydroxystearic Acid, Polyperfluoroethoxymethoxy PEG-2 Phosphate, Polyvinyl Imidazolinium Acetate, Polyvinyl Methyl Ether, PPG-3 Myristyl Ether Neoheptanoate, PVM/MA Copolymer, PVP, PVP/VA/Itaconic Acid Copolymer, Quaternium-18 Bentonite, Quaternium-18/Benzalkonium Bentonite, Quaternium-18 Hectorite, Quaternium-90 Bentonite, Rhizobian Gum, Silica, Silica Dimethicone Silylate, Silica Dimethyl Silylate, Silica Silylate, Sodium Acrylate/Acryloyldimethyl Taurate Copolymer, Sodium Acrylates/Vinyl Isodecanoate Crosspolymer, Sodium Acrylic Acid/MA Copolymer, Sodium C4-12 Olefin/Maleic Acid Copolymer, Sodium Dextran Sulfate, Sodium Dimaltodextrin Phosphate, Sodium Glycereth-1 Polyphosphate, Sodium Isooctylene/MA Copolymer, Sodium Magnesium Fluorosilicate, Starch Hydroxypropyltrimonium Chloride, Stearalkonium Bentonite, Stearalkonium Hectorite, Stearylvinyl Ether/MA Copolymer, Styrene/Acrylates/Acrylonitrile Copolymer, Styrene/Acrylates/Ammonium Methacrylate Copolymer, Styrene/MA Copolymer, Sucrose Benzoate/Sucrose Acetate Isobutyrate/Butyl Benzyl Phthalate Copolymer, Tosylamide/Epoxy Resin, Tosylamide/Formaldehyde Resin, VP/Dimethylaminoethylmethacrylate Copolymer, VP/Eicosene Copolymer, VP/Hexadecene Copolymer, VP/VA Copolymer.

It is also possible according to the invention for the preparations used to contain a plurality of, in particular two different, identically charged polymers and/or in each case one ionic and one amphoteric and/or nonionic polymer.

Further preferred polymers are all the polymers which are mentioned in the “International Cosmetic Ingredient Dictionary and Handbook”, (seventh edition 1997, The Cosmetic, Toiletry, and Fragrance Association 1101 17th Street, N.W., Suite 300, Washington, D.C. 20036-4702) in one of the chapters about polymers as polymers, such as for example “film formers” or “hair fixatives” and are commercially available. Reference is explicitly made to this document and the portions cited therefrom.

It may also be advantageous in a preferred embodiment to formulate at least one softening and/or at least one film-forming, setting polymer and/or at least one thickening polymer. Polymers should be taken to mean both natural and synthetic polymers, which may be anionically, cationically or amphoterically charged or nonionic. Thus, the polymer (G) according to the invention may be both a setting and/or film-forming polymer and a polymer with conditioning or softening and/or thickening properties.

The polymers (G) are contained in the agents used according to the invention preferably in quantities of from 0.01 to 30 wt. %, relative to the total agent. Quantities of 0.01 to 25, in particular of 0.01 to 15 wt. %, are particularly preferred.

The compositions according to the invention particularly preferably contain fatty substances (D) as a further active ingredient. In a particularly preferred embodiment, the active ingredient complex according to the invention contains a sandalwood extract, a fatty substance and a further active ingredient selected from cationic surfactants, amphoteric and/or zwitterionic surfactants, cationic polymers or amphoteric and/or zwitterionic polymers. As a result of this active ingredient combination, increased quantities of active ingredients are deposited on the hair or skin, which leads to synergistically increased effects.

Fatty substances (D) should be taken to mean fatty acids, fatty alcohols, natural and synthetic waxes, which may assume both solid form and liquid form in an aqueous dispersion, and natural and synthetic cosmetic oil components.

Fatty acids (D1) which may be used are linear and/or branched, saturated and/or unsaturated fatty acids having 6-30 carbon atoms. Fatty acids having 10-22 carbon atoms are preferred. Such substances which may, for example, be mentioned are isostearic acids, such as the commercial products Emersol® 871 and Emersol® 875, and isopalmitic acids such as the commercial product Edenor® IP 95, and any further fatty acids distributed under the tradename Edenor® (Cognis). Further typical examples of such fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and the technical mixtures thereof which are obtained, for example, on pressure splitting of natural fats and oils, on oxidation of aldehydes from Roelen's oxo synthesis or on dimerization of unsaturated fatty acids. The fatty acid cuts obtainable from coconut oil or palm oil are conventionally particularly preferred; in general, it is particularly preferred to use stearic acid.

The quantity used here amounts to 0.1-15 wt. %, relative to the total agent. Preferably, the quantity amounts to 0.5-10 wt. %, with quantities of 1-5 wt. % being very particularly advantageous.

Fatty alcohols (D2) which may be used are saturated, mono- or polyunsaturated, branched or unbranched fatty alcohols having C₆-C₃₀, preferably C₁₀-C₂₂ and very particularly preferably C₁₂-C₂₂ carbon atoms. For the purposes of the invention, it is for example possible to use decanol, octanol, octenol, dodecenol, decenol, octadienol, dodecadienol, decadienol, oleyl alcohol, erucic alcohol, ricinol alcohol, stearyl alcohol, isostearyl alcohol, cetyl alcohol, lauryl alcohol, myristyl alcohol, arachidyl alcohol, caprylic alcohol, capric alcohol, linoleyl alcohol, linolenyl alcohol and behenyl alcohol, and the Guerbet alcohols thereof, it being intended for this list to be of an exemplary rather than limiting nature. The fatty alcohols are, however, preferably derived from natural fatty acids, it conventionally being possible to start by isolation from the fatty acid esters by reduction. It is also possible to use according to the invention those fatty alcohol cuts which are produced by reducing naturally arising triglycerides such as beef fat, palm oil, peanut oil, rapeseed oil, cottonseed oil, soy oil, sunflower oil and linseed oil or fatty acids arising from the transesterification products thereof with corresponding alcohols, and thus constitute a mixture of different fatty alcohols. Such substances are commercially available for example under the names Stenol®, for example Stenol® 1618 or Lanette®, for example Lanette® 0 or Lorol®, for example Lorol® C8, Lorol® C14, Lorol® C18, Lorol® C₈₋₁₈, HD-Ocenol®, Crodacol®, for example Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. Wool wax alcohols, as are for example commercially obtainable under the names Corona®, White Swan®, Coronet® or Fluilan®, may of course also be used according to the invention. The fatty alcohols are used in quantities of 0.1-30 wt. %, relative to the total preparation, preferably in quantities of 0.1-20 wt. %.

Natural or synthetic waxes (D3) which may be used according to the invention are solid paraffins or isoparaffins, carnauba waxes, beeswaxes, candelilla waxes, ozokerites, ceresin, spermaceti, sunflower wax, fruit waxes such as for example apple wax or citrus wax, PE or PP microwaxes. Such waxes are obtainable for example through Kahl & Co., Trittau.

The quantity used amounts to 0.1-50 wt. % relative to the total preparation, preferably 0.1-20 wt. % and particularly preferably 0.1-15 wt. % relative to the total agent.

Natural and synthetic cosmetic oil bodies (D4) include, for example: vegetable oils. Examples of such oils are sunflower oil, olive oil, soy oil, rapeseed oil, almond oil, jojoba oil, orange oil, wheat germ oil, peach stone oil and the liquid fractions of coconut oil. However, other triglyceride oils such as the liquid fractions of beef fat together with synthetic triglyceride oils are also suitable liquid paraffin oils, isoparaffin oils and synthetic hydrocarbons and di-n-alkyl ethers having a total of between 12 to 36 C atoms, in particular 12 to 24 C atoms, such as for example di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether and di-tert.-butyl ether, di-iso-pentyl ether, di-3-ethyldecyl ether, tert.-butyl-n-octyl ether, iso-pentyl-n-octyl ether and 2-methylpentyl-n-octyl ether. The compounds 1,3-di-(2-ethylhexyl)cyclohexane (Cetiol® S) and di-n-octyl ether (Cetiol® OE) available as commercial products may be preferred ester oils. Ester oils should be taken to mean the esters of C₆-C₃₀ fatty acids with C₂-C₃₀ fatty alcohols. The monoesters of fatty acids with alcohols having 2 to 24 C atoms are preferred. Examples of fatty acid moieties used in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and the technical mixtures thereof which are obtained, for example, on pressure splitting of natural fats and oils, on oxidation of aldehydes from Roelen's oxo synthesis or on dimerization of unsaturated fatty acids. Examples of fatty alcohol moieties in the ester oils are isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and the technical mixtures thereof, which are obtained, for example, on high pressure hydrogenation of technical methyl esters based on fats and oils or aldehydes from Roelen's oxo synthesis and as the monomer fraction on dimerization of unsaturated fatty alcohols. Particularly preferred substances according to the invention are isopropyl myristate (Rilanit® IPM), isononanoic acid C16-18 alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V), dicarboxylic acid esters such as di-n-butyl adipate, di-(2-ethylhexyl) adipate, di-(2-ethylhexyl) succinate and diisotridecyl acelate and diol esters such as ethylene glycol dioleate, ethylene glycol diisotridecanoate, propylene glycol di(2-ethyl hexanoate), propylene glycol diisostearate, propylene glycol dipelargonate, butanediol diisostearate, neopentyl glycol dicaprylate, symmetrical, asymmetrical or cyclic esters of carbonic acid with fatty alcohols, for example described in DE-OS 197 56 454, glycerol carbonate or dicaprylyl carbonate (Cetiol® CC), trifatty acid esters of saturated and/or unsaturated linear and/or branched fatty acids with glycerol, fatty acid partial glycerides, i.e. monoglycerides, diglycerides and the technical mixtures thereof. When using technical products, small quantities of triglycerides may still be contained therein, depending on the production method. Partial glycerides preferably follow the formula (D4-I),

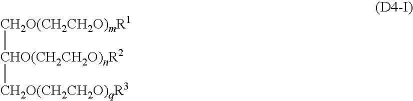

in which R¹, R² and R³ mutually independently denote hydrogen or a linear or branched, saturated and/or unsaturated acyl residue with 6 to 22, preferably 12 to 18, carbon atoms, with the proviso that at least one of these groups denotes an acyl residue and at least one of these groups denotes hydrogen. The sum (m+n+q) denotes 0 or numbers from 1 to 100, preferably 0 or 5 to 25. Preferably, R¹ denotes an acyl residue and R² and R³ denote hydrogen and the sum (m+n+q) is 0. Typical examples are mono- and/or diglycerides based on caproic acid, caprylic acid, 2-ethylhexanoic acid, capric acid, lauric acid, isotridecanoic acid, myristic acid, palmitic acid, palmoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and the technical mixtures thereof. Preferably, oleic acid monoglycerides are used.

The input quantity of the natural and synthetic cosmetic oil bodies in the agents used according to the invention conventionally amounts to 0.1-30 wt. % relative to the total agent, preferably 0.1-20 wt. %, and in particular 0.1-15 wt. %.

One last group of substances which may be used as fatty substances is silicones.

A further class of substances, which is contained as an active ingredient in the compositions according to the invention as an alternative to those described above, comprises silicone oils (S). Silicone oils bring about the most varied effects. For example, they simultaneously influence dry and wet combability, the handle of dry and wet hair and gloss. However, the softness and elasticity of the film formed by film-forming polymers on the hair for the purpose of setting and styling, is positively influenced by silicones. The term silicone oils is understood by the person skilled in the art to mean a plurality of organosilicon compounds of different structures. The first among these are the dimethiconols (S1). Dimethiconols form the first group of silicones which are particularly preferred according to the invention. The dimethiconols according to the invention may be both linear and branched and cyclic or cyclic and branched. Linear dimethiconols may be illustrated by the following structural formula (S1-I):

(SiOHR¹₂)—O—(SiR²₂—O—)ₓ—(SiOHR¹₂) (S1-I)

Branched dimethiconols may be illustrated by the structural formula (S1-II):

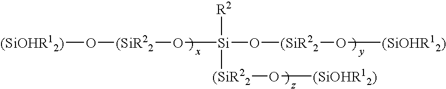

The residues R¹ and R² mutually independently denote in each case hydrogen, a methyl residue, a C₂ to C₃₀ linear, saturated or unsaturated hydrocarbon residue, a phenyl residue and/or an aryl residue. Non-limiting examples of the residues represented by R¹ and R² include alkyl residues, such as methyl, ethyl, propyl, isopropyl, butyl, isobutyl, pentyl, isopentyl, neopentyl, amyl, isoamyl, hexyl, isohexyl and the like; alkenyl residues, such as vinyl, halovinyl, alkylvinyl, allyl, haloallyl, alkylallyl; cycloalkyl residues, such as cyclobutyl, cyclopentyl, cyclohexyl and the like; phenyl residues, benzyl residues, halogenated hydrocarbon residues, such as 3-chloropropyl, 4-bromobutyl, 3,3,3-trifluoropropyl, chlorocyclohexyl, bromophenyl, chlorophenyl and the like and sulfur-containing residues, such as mercaptoethyl, mercaptopropyl, mercaptohexyl, mercaptophenyl and the like; R¹ and R² are preferably an alkyl residue containing 1 to approx. 6 carbon atoms, and R¹ and R² are most preferably methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂ CH₂CH₂—, —CH₂CH(CH₃)C(O)OCH₂—, —(CH₂)₃ CC(O)OCH₂CH₂—, —C₆H₄C₆H₄—, —C₆H₄CH₂C₆H₄—; and —(CH₂)₃C(O)SCH₂CH₂—. R¹ and R² are preferably methyl, phenyl and C₂ to C₂₂ alkyl residues. The C₂ to C₂₂ alkyl residues are very particularly preferably lauryl, stearyl and behenyl residues. The numbers x, y and z are integers and run in each case mutually independently from 0 to 50000. The molar weights of the dimethicones are between 1000 D and 10000000 D. Viscosities are between 100 and 10000000 cPs measured at 25° C. with the assistance of a glass capillary viscometer using the Dow Corning Corporate Test Method CTM 0004 of 20 Jul. 1970. Preferred viscosities are between 1000 and 5000000 cPs, very particularly preferred viscosities are between 10000 and 3000000 cPs. The most preferred range is between 50000 and 2000000 cPs.

Of course, the teaching according to the invention also provides that the dimethiconols may already be present as an emulsion. In this case, the corresponding dimethiconol emulsion may be produced both after the production of the corresponding dimethiconols from the latter and using the conventional methods of emulsification known to a person skilled in the art. To this end, any of cationic, anionic, nonionic or zwitterionic surfactants and emulsifiers may be used as auxiliary materials for producing the corresponding emulsions. It goes without saying that the dimethiconol emulsions may also be produced directly by an emulsion polymerization method. Such methods are also well known to a person skilled in the art. In this respect, reference is made for example to the “Encyclopedia of Polymer Science and Engineering”, Volume 15, Second Edition, pages 204 to 308, John Wiley & Sons., Inc. 1989. Reference is explicitly made to this standard work.

If the dimethiconols according to the invention are used in the form of an emulsion, the droplet size of the emulsified particles then amounts according to the invention to 0.01 μm to 10000 μm, preferably 0.01 to 100 μm, very particularly preferably 0.01 to 20 μm and most preferably 0.01 to 10 μm. Particle size is here determined using the light scattering method.

If branched dimethiconols are used, it should be understood that the branching is greater in this case than the chance branching which arises due to impurities in the respective monomers. For the purposes of the present compound, branched dimethiconols should therefore be taken to mean that the degree of branching is greater than 0.01%. Preferably, the degree of branching is greater than 0.1% and very particularly preferably greater than 0.5%. The degree of branching is determined in this case from the ratio of unbranched monomers, i.e. the quantity of monofunctional siloxane, to the branched monomers, i.e. the quantity of tri- and tetrafunctional siloxanes. According to the invention, dimethiconols with both a low and a high degree of branching may be very particularly preferred.

The following commercial products can be mentioned as examples of such products: Botanisil NU-150M (Botanigenics), Dow Corning 1-1254 fluid, Dow Corning 2-9023 fluid, Dow Corning 2-9026 fluid, Ultrapure Dimethiconol (Ultra Chemical), Unisil SF-R (Universal Preserve), X-21-5619 (Shin-Etsu Chemical Co.), Abil OSW 5 (Degussa Care Specialties), ACC DL-9430 Emulsion (Taylor Chemical Company), AEC Dimethiconol & Sodium Dodecylbenzenesulfonate (A & E Connock (Perfumery & Cosmetics) Ltd.), B C Dimethiconol Emulsion 95 (Basildon Chemical Company, Ltd.), Cosmetic Fluid 1401, Cosmetic Fluid 1403, Cosmetic Fluid 1501, Cosmetic Fluid 1401DC (all above-stated from Chemsil Silicones, Inc.), Dow Corning 1401 fluid, Dow Corning 1403 fluid, Dow Corning 1501 fluid, Dow Corning 1784 HVF Emulsion, Dow Corning 9546 Silicone Elastomer Blend (all above-stated from Dow Corning Corporation), Dub Gel SI 1400 (Stearinerie Dubois Fils), HVM 4852 Emulsion (Crompton Corporation), Jeesilc 6056 (Jeen International Corporation), Lubrasil, Lubrasil DS (both from Guardian Laboratories), Nonychosine E, Nonychosine V (both from Exsymol), SanSurf Petrolatum-25, Satin Finish (both from Collaborative Laboratories, Inc.), Silatex-D30 (Cosmetic Ingredient Resources), Silsoft 148, Silsoft E-50, Silsoft E-623 (all above-stated from Crompton Corporation), SM555, SM2725, SM2765, SM2785 (all above-stated from GE Silicones), Taylor T-Sil CD-1, Taylor TME-4050E (all from Taylor Chemical Company), TH V 148 (Crompton Corporation), Tixogel CYD-1429 (Süd-Chemie Performance Additives), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (all above-stated from Wacker-Chemie GmbH).

The dimethiconols (S1) are in the compositions according to the invention in quantities of 0.01 to 10 wt. %, preferably 0.01 to 8 wt. %, particularly preferably 0.1 to 7.5 wt. % and in particular 0.1 to 5 wt. % of dimethiconol relative to the composition.

It is also possible according to the invention for the dimethiconols to form their own phase in the compositions according to the invention. In this case it may be appropriate for the composition to be briefly homogenized by shaking immediately before application. In this case, the quantity of dimethiconol may amount to up to 40 wt. %, preferably in quantities of up to 25 wt. % relative to the total composition.

Dimethicones (S2) form the second group of silicones which are particularly preferred according to the invention. The dimethicones according to the invention may be both linear and branched and cyclic or cyclic and branched. Linear dimethicones may be illustrated by the following structural formula (S2-I):

(SiR¹₃)—O—(SiR²₂—O—)ₓ—(SiR¹₃) (S2-I)

Branched dimethicones may be illustrated by the structural formula (S2-II):

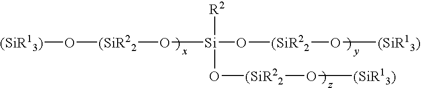

The residues R¹ and R² mutually independently denote in each case hydrogen, a methyl residue, a C₂ to C₃₀ linear, saturated or unsaturated hydrocarbon residue, a phenyl residue and/or an aryl residue. Non-limiting examples of the residues represented by R¹ and R² include alkyl residues, such as methyl, ethyl, propyl, isopropyl, butyl, isobutyl, pentyl, isopentyl, neopentyl, amyl, isoamyl, hexyl, isohexyl and the like; alkenyl residues, such as vinyl, halovinyl, alkylvinyl, allyl, haloallyl, alkylallyl; cycloalkyl residues, such as cyclobutyl, cyclopentyl, cyclohexyl and the like; phenyl residues, benzyl residues, halogenated hydrocarbon residues, such as 3-chloropropyl, 4-bromobutyl, 3,3,3-trifluoropropyl, chlorocyclohexyl, bromophenyl, chlorophenyl and the like and sulfur-containing residues, such as mercaptoethyl, mercaptopropyl, mercaptohexyl, mercaptophenyl and the like; R¹ and R² are preferably an alkyl residue containing 1 to approx. 6 carbon atoms, and R¹ and R² are most preferably methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—; and —(CH₂)₃C(O)SCH₂CH₂—. R¹ and R² are preferably methyl, phenyl and C₂ to C₂₂ alkyl residues. The C₂ to C₂₂ alkyl residues are very particularly preferably lauryl, stearyl and behenyl residues. The numbers x, y and z are integers and run in each case mutually independently from 0 to 50000. The molar weights of the dimethicones are between 1000 D and 10000000 D. Viscosities are between 100 and 10000000 cPs measured at 25° C. with the assistance of a glass capillary viscometer using the Dow Corning Corporate Test Method CTM 0004 of 20 Jul. 1970. Preferred viscosities are between 1000 and 5000000 cPs, very particularly preferred viscosities are between 10000 and 3000000 cPs. The most preferred range is between 50000 and 2000000 cPs.

Of course, the teaching according to the invention also provides that the dimethicones may already be present as an emulsion. In this case, the corresponding dimethicone emulsion may be produced both after the production of the corresponding dimethicones from the latter and using the conventional methods of emulsification known to a person skilled in the art. To this end, any of cationic, anionic, nonionic or zwitterionic surfactants and emulsifiers may be used as auxiliary materials for producing the corresponding emulsions. It goes without saying that the dimethicone emulsions may also be produced directly by an emulsion polymerization method. Such methods are also well known to a person skilled in the art. In this respect, reference is made for example to the “Encyclopedia of Polymer Science and Engineering”, Volume 15, Second Edition, pages 204 to 308, John Wiley & Sons., Inc. 1989. Reference is explicitly made to this standard work.

If the dimethicones according to the invention are used in the form of an emulsion, the droplet size of the emulsified particles then amounts according to the invention to 0.01 μm to 10000 μm, preferably 0.01 to 100 μm, very particularly preferably 0.01 to 20 μm and most preferably 0.01 to 10 μm. Particle size is here determined using the light scattering method.

If branched dimethicones are used, it should be understood that the branching is greater in this case than the chance branching which arises due to impurities in the respective monomers. For the purposes of the present compound, branched dimethicones should therefore be taken to mean that the degree of branching is greater than 0.01%. Preferably, the degree of branching is greater than 0.1% and very particularly preferably greater than 0.5%. The degree of branching is determined in this case from the ratio of unbranched monomers, i.e. the quantity of monofunctional siloxane, to the branched monomers, i.e. the quantity of tri- and tetrafunctional siloxanes. According to the invention, dimethicones with both a low and a high degree of branching may be very particularly preferred.

The dimethicones (S2) are in the compositions according to the invention in quantities of 0.01 to 10 wt. %, preferably 0.01 to 8 wt. %, particularly preferably 0.1 to 7.5 wt. % and in particular 0.1 to 5 wt. % of dimethicones relative to the composition.

It is also possible according to the invention for the dimethicones to form their own phase in the compositions according to the invention. In this case it may be appropriate for the composition to be briefly homogenized by shaking immediately before application. In this case, the quantity of dimethicone may amount to up to 40 wt. %, preferably in quantities of up to 25 wt. % relative to the total composition.

It goes without saying that the teaching according to the invention also includes the fact that a mixture of a plurality of fatty substances (D) from different classes of fatty substances, at least two different fatty substance classes, may be used in the compositions according to the invention. The preferred mixtures of at least two oil and fat components in this case have to contain at least one further silicone component. Preferably, the silicone component is selected in this case from the dimethiconols and the amodimethicones.

The total quantity of oil and fat components in the agents according to the invention conventionally amounts to 0.5-75 wt. %, relative to the total agent. Quantities of 0.5-35 wt. % are preferred according to the invention.

A further synergistic active ingredient according to the invention in the compositions according to the invention comprises protein hydrolyzates and/or the derivatives thereof (P).

Proteins and/or protein hydrolyzates are capable of significantly restructuring the inner structure of fibers, in particular keratin fibers. Structure strengthening, i.e. restructuring for the purposes of the invention, is understood to mean reducing the damage to keratin fibers arising as a result of the widest possible range of influences. A significant role is played here, for example, by the restoration of natural strength. Restructured fibers are distinguished for example by improved gloss or by improved handle or by easier combability. In addition, they display optimized strength and elasticity. Successful restructuring may be identified physically as an increase in melting point when compared with damaged fibers. The higher the melting point of the hair, the stronger the structure of the fibers. A precise description of the method of determining the melting range of hair is to be found in DE 196 173 95 A1.

Proteins and protein hydrolyzates have long been known and are often used in cosmetics. In this regard, reference should be made to the relevant specialist literature, for example A. Domsch, “Die kosmetischen Präparate” [cosmetic preparations], volume II, page 205 et. seq., Verlag für die chemische Industrie, H. Ziolkowsky.

It has long been known to use proteins or indeed modified proteins in cosmetic preparations to achieve care effects. To this end, either water-soluble proteins or proteins modified by chemical and/or by enzymatic reactions, i.e. water-solubilized proteins, are used. It is precisely during reactions to achieve sufficient water solubility that fibrous proteins have to undergo such extensive degradation that cosmetic efficacy is no longer sufficient.

In this way, in particular, an increase in mildness and in skin compatibility is achieved, as well, if desired, as a fine, creamy foam when applying the compositions according to the invention. This structurally very fine and creamy foam, which feels extremely pleasant, is achieved in all compositions in which in particular surface-active substances are contained as further constituents. The efficacy of the composition according to the invention is further increased by the simultaneous use of polymers and/or penetration and swelling aids. In such cases, even after application of the respective composition markedly more protein hydrolyzate or the derivative thereof remains on the surface of the hair, which results in improved action. The hair is thereby markedly strengthened in structure and smoothed. This effect may also be unambiguously identified using objective action tests, such as for example measuring the combing strength of the wet and dry hair, measuring ultimate tensile strength or measuring the torsion angle on the skin. These results are also confirmed by the results of consumer tests.

Protein hydrolyzates are product mixtures which are obtained by acidically, basically or enzymatically catalyzed degradation of proteins. According to the invention, the term protein hydrolyzates also covers total hydrolyzates and individual amino acids and the derivatives thereof and mixtures of different amino acids. Furthermore, polymers built up from amino acids and amino acid derivatives are also covered according to the invention by the term protein hydrolyzates. The latter include for example polyalanine, polyasparagine, polyserine etc. Further examples of compounds which may be used according to the invention are L-alanyl-L-proline, polyglycine, glycyl-L-glutamine or D/L-methionine-S-methylsulfonium chloride. It goes without saying that, according to the invention, β-amino acids and the derivatives thereof such as β-alanine, anthranilic acid or hippuric acid may also be used. The molecular weight of the protein hydrolyzates which may be used according to the invention is between 75, the molecular weight of glycine, and 200000, the molecular weight preferably amounting to 75 to 50000 and very particularly preferably to 75 to 20000 daltons. It goes without saying that the present teaching according to the invention also includes the fact that, in the case of amino acids, the latter may be present in the form of derivatives, such as for example N-acyl derivatives, N-alkyl or O-esters. In the case of N-acyl derivatives, the acyl group is a formyl residue, an acetyl residue, a propionyl residue, a butyryl residue or the residue of a straight-chain, branched or unbranched, saturated or unsaturated fatty acid with a chain length of 8 to 30 C atoms. In the case of an N-alkyl derivative, the alkyl group may be linear, branched, saturated or unsaturated and has a C-chain length of 1 to 30 C atoms. In the case of O-esters, the alcohols underlying esterification are methanol, ethanol, isopropanol, propanol, butanol, isobutanol, pentanol, neopentanol, isopentanol, hexanols, heptanols, caprylic or caproic alcohol, octanols, nonanols, decanols, dodecanols, lauranols, in particular saturated or unsaturated, linear or branched, alcohols with a C chain length of 1 to 30 C atoms. It goes without saying that the amino acids may be derivatized at the same time both on the N atom and the O atom. It goes without saying that the amino acids may also be used in salt form, in particular as mixed salts together with edible acids. This may be preferred according to the invention.

The following examples of amino acids and the derivatives thereof as protein hydrolyzates according to the invention are mentioned: alanine, arginine, carnitine, creatine, cystathionine, cysteine, cystine, cystine acid, glycine, histidine, homocysteine, homoserine, isoleucine, lanthionine, leucine, lysine, methionine, norleucine, norvaline, ornithine, phenylalanine, proline, hydroxyproline, sarcosine, serine, threonine, tryptophan, thyronine, tyrosine, valine, aspartic acid, asparagine, glutamic acid and glutamine. Preferred amino acids are alanine, arginine, glycine, histidine, lanthionine, leucine, lysine, proline, hydroxyproline, serine and asparagine. Use is very particularly preferably made of alanine, glycine, histidine, lysine, serine and arginine. Glycine, histidine, lysine and serine are most preferably used.

Protein hydrolyzates of both plant and animal origin or marine or synthetic origin may be used according to the invention.

Animal protein hydrolyzates are for example elastin, collagen, keratin, silk and milk protein hydrolyzates which may also assume salt form. Such products are distributed for example under the tradenames Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

Recently, in particular plant proteins and the hydrolyzates and derivatives thereof have been used ever more frequently in cosmetics. Products based on wheat, oats, rice, maize, potatoes or soy are known, for example. Plants which contain active constituents of interest also include the Moringa plant family, of which there are approximately 14 species. One of these is Moringa oleifera (Moringa pterygosperma). Further species are for example Moringa drouhardii, Moringa concanensis or Moringa peregrina. It is known for example from U.S. Pat. No. 6,667,047 B2 to use the oil from these species. It is not previously known, however, to use an extract of the Moringa oleifera seed. Extraction of this seed with a water/glycerol mixtures gives rise to an extract, which consists of proteins with a molecular weight of for instance 500 to 50000 Dalton. Such a protein is commercially available for example from Laboratoires Sérobiologiques under the tradename Puricare® LS 9658.

Moringa plants have been known since ancient times. Plants of this species are better known under their common name “tree of miracles”. They are indigenous to tropical regions. The various parts of this plant genus have been used since ancient times in particular for medical purposes. Protein is isolated from the seeds of the Moringa plant by careful extraction with water and glycerol. This protein has a molecular weight of 500 to 50000 Dalton. A protein extract with a molecular weight of 3000 to 30000 Dalton is preferred, with one of 5000 to 15000 Dalton being very particularly preferred. The most preferred extract is isolated from the Moringa oleifera plant. The extract according to the invention of course additionally contains water and glycerol, due to the extraction process. The content of extracted protein in the extract amounts to 0.01 to 20 wt. %. A protein content of 0.01 to 10 wt. % is preferred. Particular preference is given to an extract with a protein content of 0.01 to 5 wt. %. Furthermore, at least 30 wt. % of glycerol is contained in the extract. Finally, water is contained in the extract according to the invention.

In cosmetic compositions the above-described protein extract from the seeds of the Moringa plant is contained in a quantity of at least 0.01 to 20 wt. %. The quantities used of the extract are preferably 0.01 up to 10 wt. %, very particularly preferably 0.01 to 5 wt. % relative to the total cosmetic composition.

Further plant protein hydrolyzates preferred according to the invention are for example soy, almond, pea, potato and wheat protein hydrolyzates. Such products are obtainable for example under the trademarks Gluadin® (Cognis), DiaMin® (Diamalt), Lexein® (Inolex), Hydrosoy® (Croda), Hydrolupin® (Croda), Hydrosesame® (Croda), Hydrotritium® (Croda) and Crotein® (Croda).

Further protein hydrolyzates preferred according to the invention are of maritime origin. These include for example collagen hydrolyzates from fish or algae and protein hydrolyzates from mussels or pearl hydrolyzates.

Mussel pearls substantially consist of inorganic and organic calcium salts, trace elements and proteins. Pearls may straightforwardly be obtained from cultured mussels. Mussels can be cultured in both fresh water and seawater. This may have an impact on the constituents of the pearls. A pearl extract which is preferred according to the invention is one which originates from mussels cultured in seawater or salt water. The pearls consist to a great extent of aragonite (calcium carbonate), conchiolin and an albuminoid. The latter constituents are proteins. Pearls furthermore additionally contain magnesium and sodium salts, inorganic silicon compounds and phosphates.

Pearl extract is produced by pulverizing the pearls. The pulverized pearls are then extracted using conventional methods. Extracting agents for producing the pearl extracts may comprise water, alcohols and mixtures thereof. Water should here be taken to mean both demineralized water and seawater. Preferred alcohols are lower alcohols such as ethanol and isopropanol, but in particular polyhydric alcohols such as glycerol, diglycerol, triglycerol, polyglycerol, ethylene glycol, propylene glycol and butylene glycol, both as sole extracting agent and in a mixture with demineralized water or sea water. Pearl extracts based on water/glycerol mixtures have proved particularly suitable. Depending on the extraction conditions, the pearl proteins (conchiolin and albuminoid) may be present very largely in their native state or already partially or very largely as protein hydrolyzates. A preferred pearl extract is one in which conchiolin and albuminoid already assume partially hydrolyzed form. The essential amino acids of these proteins are glutamic acid, serine, alanine, glycine, aspartic acid and phenylalanine. In a further particularly preferred development, it may be advantageous for the pearl extract additionally to be enriched with at least one or more of these amino acids. In the most preferred embodiment, the pearl extract is enriched with glutamic acid, serine and leucine.

Furthermore, depending on the extraction conditions, in particular depending on the extracting agent selected, the extract contains a greater or lesser proportion of minerals and trace elements. One preferred extract contains organic and/or inorganic calcium salts and magnesium and sodium salts, inorganic silicon compounds and/or phosphates. One very particularly preferred pearl extract contains at least 75%, preferably 85%, more preferably 90% and very particularly preferably 95% of all the constituents of the naturally occurring pearls.

Examples of pearl extracts according to the invention are the commercial products Pearl Protein Extract BG® or Crodarom® Pearl.

In cosmetic compositions one of the above-described pearl extracts is contained in a quantity of at least 0.01 to 20 wt. %. The quantities used of the extract are preferably 0.01 up to 10 wt. %, very particularly preferably 0.01 to 5 wt. % relative to the total cosmetic composition.

A further very particular protein hydrolyzate is isolated from silk.

Silk is a fibrous protein of great cosmetic interest. Silk is taken to mean the fibers of the cocoon of the silkworm (Bombyx mori L). Raw silk fiber consists of a double filament fibroin. Sericin acts as the cement holding this double filament together. Silk is made up of 70-80 wt. % fibroin, 19-28 wt. % sericin, 0.5-1 wt. % fat and 0.5-1 wt. % colors and mineral constituents.

Hydroxyamino acids, accounting for approx. 46 wt. %, are the substantial constituents of sericin. Sericin consists of a group of 5 to 6 proteins. The substantial amino acids of sericin are serine (Ser, 37 wt. %), aspartate (Asp, 26 wt. %), glycine (Gly, 17 wt. %), alanine (Ala), leucine (Leu) and tyrosine (Tyr).

Fibroin, which is insoluble in water, should be classed among scleroproteins with a long-chain molecular structure. The main constituents of fibroin are glycine (44 wt. %), alanine (26 wt. %) and tyrosine (13 wt. %). Another substantial structural feature of fibroin is the hexapeptide sequence Ser-Gly-Ala-Gly-Ala-Gly.

It is technically straightforward to separate the two silk proteins from one another. It is thus not surprising that both sericin and fibroin are in each case individually known as raw materials for use in cosmetic products. Protein hydrolyzates and derivatives based on the individual silk proteins are furthermore known raw materials in cosmetics. For example, sericin as such is distributed by Pentapharm Ltd. as a commercial product known as Sericin Code 303-02. Fibroin is still more frequently offered for sale as a protein hydrolyzate with various molecular weights. These hydrolyzates are in particular understood to be “silk hydrolyzates”. Hydrolyzed fibroin with average molecular weights of between 350 and 1000 is, for example, accordingly distributed under the tradename Promois® Silk. DE 31 39 438 A1 describes colloidal fibroin solutions as an additive in cosmetics.

The positive properties of the silk protein derivatives of sericin and fibroin are in each case per se known in the literature. The sales brochure for Pentapharm accordingly describes the cosmetic effects of sericin on the skin as irritation-relieving, hydrating and film-forming. The properties of a shampoo containing sericin as a hair care component are presented in “Ärztlichen Kosmetologie [medical cosmetology] 17, 91-110 (1987)” by W. Engel et al. A fibroin derivative is described, for example in DE 31 39 438 A1, as having a conditioning and softening action on hair.

The following may preferably be used according to the invention as active ingredients:

native sericin,
hydrolyzed and/or further derivatized sericin, such as for example commercial products with the INCI names Sericin, Hydrolyzed Sericin, or Hydrolyzed Silk,
a mixture of the amino acids serine, aspartate and glycine and/or the methyl, propyl, iso-propyl, butyl, iso-butyl esters thereof, the salts thereof such as for example hydrochlorides, sulfates, acetates, citrates, tartrates, this mixture containing 20 to 60 wt. % serine and/or derivatives thereof, 10-40 wt. % aspartate and/or derivatives thereof and 5 to 30 wt. % glycine and/or derivatives thereof, with the proviso, that the quantities of these amino acids and/or the derivatives thereof preferably make 100 wt. %, and mixtures thereof.

According to the invention the following may additionally be used as active ingredients:

native fibroin converted into a soluble form,
hydrolyzed and/or further derivatized fibroin, in particular partially-hydrolyzed fibroin, which contains the amino acid sequence Ser-Gly-Ala-Gly-Ala-Gly as the main constituent, the amino acid sequence Ser-Gly-Ala-Gly-Ala-Gly,
a mixture of the amino acids glycine, alanine and tyrosine and/or the methyl, propyl, iso-propyl, butyl, iso-butyl esters thereof, the salts thereof such as for example hydrochlorides, sulfates, acetates, citrates, tartrates, this mixture containing 20 to 60 wt. % glycine and/or derivatives thereof, 10-40 wt. % alanine and derivatives thereof and 0 to 25 wt. % tyrosine and derivatives thereof, with the proviso that the quantities of these amino acids and/or the derivatives thereof preferably add up to 100 wt. %,
and mixtures thereof.

If both silk protein hydrolyzates and/or the derivatives thereof are used simultaneously in the compositions according to the invention of the agent according to the invention, it may be preferred according to the invention for at least one of the two silk constituents, fibroin or sericin, to be used in the native or if need be solubilized form. According to the invention, it is also possible to use a mixture of a plurality of silk protein hydrolyzates or the derivatives thereof.

If a mixture of at least two silk hydrolyzates and/or the derivatives thereof is used, it may be preferred according to the invention for the two silk protein hydrolyzates to be used in the ratio of 10:90 to 70:30, in particular 15:85 to 50:50 and very particularly 20:80 to 40:60, relative to the respective contents of active substance in the preparations according to the invention.

The derivatives of hydrolyzates of sericin and fibroin comprise both anionic and cationized protein hydrolyzates. The protein hydrolyzates of sericin and fibroin according to the invention and the derivatives produced therefrom may be obtained from the corresponding proteins by chemical, in particular alkaline or acidic, hydrolysis, by enzymatic hydrolysis and/or a combination of both types of hydrolysis. Protein hydrolysis as a rule gives rise to a protein hydrolyzate with a molecular weight distribution of approx. 100 daltons up to several thousand daltons. Preferred protein hydrolyzates of sericin and fibroin and/or the derivatives thereof are those, the underlying protein moiety of which has a molecular weight of 100 up to 25000 daltons, preferably 250 to 10000 daltons. Cationic protein hydrolyzates of sericin and fibroin should furthermore also be taken to mean quaternized amino acids and mixtures thereof. Quaternization of the protein hydrolyzates or of the amino acids is often performed by means of quaternary ammonium salts such as for example N,N-dimethyl-N-(n-alkyl)-N-(2-hydroxy-3-chloro-n-propyl)-ammonium halides. The cationic protein hydrolyzates may additionally also be still further derivatized. Typical examples of the cationic protein hydrolyzates and derivatives according to the invention which may be mentioned are those that are commercially obtainable and mentioned under the INCI names in the “International Cosmetic Ingredient Dictionary and Handbook”, (seventh edition 1997, The Cosmetic, Toiletry, and Fragrance Association 1101 17th Street, N.W., Suite 300, Washington, D.C. 20036-4702: Cocodimonium Hydroxypropyl Hydrolyzed Silk, Cocodimonium Hydroxypropyl Silk Amino Acids, Hydroxypropyltrimonium Hydrolyzed Silk, Lauryldimonium Hydroxypropyl Hydrolyzed Silk, Steardimonium Hydroxypropyl Hydrolyzed Silk, Quaternium-79 Hydrolyzed Silk. Typical examples of the anionic protein hydrolyzates and derivatives according to the invention which may be mentioned are those that are commercially obtainable and mentioned under the INCI names in the “International Cosmetic Ingredient Dictionary and Handbook”, (seventh edition 1997, The Cosmetic, Toiletry, and Fragrance Association 1101 17th Street, N.W., Suite 300, Washington, D.C. 20036-4702): Potassium Cocoyl Hydrolyzed Silk, Sodium Lauroyl Hydrolyzed Silk or Sodium Stearoyl Hydrolyzed Silk. Finally, typical examples of the derivatives of sericin and fibroin usable according to the invention which may be mentioned are the commercially obtainable products known by the INCI names: Ethyl Ester of Hydrolyzed Silk and Hydrolyzed Silk PG-Propyl Methylsilanediol. It is furthermore possible to make use according to the invention, although not necessarily preferably, of the commercially available products with the INCI names Palmitoyl Oligopeptide, Palmitoyl Pentapeptide-3, Palmitoyl Pentapeptide-2, Acetyl Hexapeptide-1, Acetyl Hexapeptide-3, Copper Tripeptide-1, Hexapeptide-1, Hexapeptide-2, MEA-Hydrolyzed Silk.

The agents used according to the invention contain the silk protein hydrolyzates and/or the derivatives thereof in quantities of 0.001-10 wt. % relative to the total agent. Quantities of 0.005 to 5, in particular 0.01 to 3 wt. %, are very particularly preferred.

Although the use of protein hydrolyzates as such is preferred, amino acid mixtures obtained in other ways may also optionally be used in their stead. It is likewise possible to use derivatives of protein hydrolyzates, for example in the form of the fatty acid condensation products thereof. Such products are distributed, for example, under the names Lamepon® (Cognis), Lexein® (Inolex), Crolastin® (Croda) or Crotein® (Croda).

A further group of protein hydrolyzates according to the invention are therefore cationically derivatized protein hydrolyzates, wherein the underlying protein hydrolyzate may originate from animals, for example from collagen, milk or keratin, from plants, for example from wheat, maize, rice, potatoes, soy or almonds, from marine life forms, for example from fish collagen or algae, or biotechnologically obtained protein hydrolyzates. The protein hydrolyzates underlying the cationic derivatives according to the invention may be obtained from the corresponding proteins by chemical, in particular alkaline or acidic, hydrolysis, by enzymatic hydrolysis and/or by a combination of both types of hydrolysis. Protein hydrolysis as a rule gives rise to a protein hydrolyzate with a molecular weight distribution of approx. 100 daltons up to several thousand daltons. Those cationic protein hydrolyzates are preferred whose underlying protein fraction has a molecular weight of 100 up to 25000 daltons, preferably 250 to 5000 daltons. Furthermore, cationic protein hydrolyzates include quaternized amino acids and mixtures thereof. Quaternization of the protein hydrolyzates or of the amino acids is often performed by means of quaternary ammonium salts such as for example N,N-dimethyl-N-(n-alkyl)-N-(2-hydroxy-3-chloro-n-propyl)-ammonium halides. The cationic protein hydrolyzates may additionally also be still further derivatized. Typical examples of the cationic protein hydrolyzates and derivatives according to the invention which may be mentioned are those that are commercially obtainable and mentioned under the INCI names in the “International Cosmetic Ingredient Dictionary and Handbook”, (seventh edition 1997, The Cosmetic, Toiletry, and Fragrance Association 1101 17th Street, N.W., Suite 300, Washington, D.C. 20036-4702: Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Soy Protein, Cocodimonium Hydroxypropyl Hydrolyzed Wheat Protein, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Soy Protein, Quaternium-79 Hydrolyzed Wheat Protein.

Plant-based cationic protein hydrolyzates and derivatives are very particularly preferred.

It goes without saying that the teaching according to the invention comprises all isomeric forms, such as cis-trans isomers, diastereomers and chiral isomers.

According to the invention, it is also possible to use a mixture of a plurality of protein hydrolyzates (P).

The protein hydrolyzates (P) are contained in the agents in concentrations of 0.001 wt. % to 20 wt. %, preferably of 0.05 wt. % to 15 wt. % and very particularly preferably in quantities of 0.05 wt. % to 5 wt. %.

The action of the compositions according to the invention may be additionally increased by a 2-pyrrolidinone-5-carboxylic acid and the derivatives thereof (J). The present invention accordingly also provides the use of derivatives of 2-pyrrolidinone-5-carboxylic acid. The sodium, potassium, calcium, magnesium or ammonium salts, in which, in addition to hydrogen, the ammonium ion bears one to three C₁ to C₄ alkyl groups, are preferred. The sodium salt is very particularly preferred. The quantities used in the agents according to the invention amount to 0.05 to 10 wt. %, relative to the total agent, particularly preferably 0.1 to 5, and in particular 0.1 to 3 wt. %.

A further preferred group of constituents, which may be used outstandingly with the composition according to the invention, comprises vitamins, provitamins or vitamin precursors.

Vitamins, provitamins and vitamin precursors are particularly preferred, which are assigned to groups A, B, C, E, F and H.

Treatment with these very particularly preferred components leaves the skin, skin being understood of course as including the scalp, with an essentially more cared-for, more vital, stronger appearance, with significantly better gloss and a very good handle both in the wet and in the dry state. This active ingredient additionally influences the regeneration and restructuring of tired skin and stressed hair, leads to regulation of the fat balance, such that the skin treated therewith and the hair become greasy again more slowly and do not tend to become over-greasy. In addition, this active ingredient has an antiinflammatory and skin-calming effect. Finally, split hair is regenerated and repaired again by these active ingredients. These active ingredients are capable of penetrating into the hair and strengthening and repairing the hair from the inside out. This “repair effect” can be objectively identified using DSC measurements. These effects may also be detected subjectively during consumer testing, for example.

The group of substances designated vitamin A includes retinol (vitamin A₁) and 3,4-didehydroretinol (vitamin A₂). β-Carotene is the provitamin of retinol. Examples of substances which may be considered according the invention as the vitamin A component are vitamin A acid and the esters thereof, vitamin A aldehyde and vitamin A alcohol and the esters thereof such as the palmitate and the acetate. The agents according to the invention preferably contain the vitamin A component in quantities of from 0.05-1 wt. %, relative to the total preparation.

The vitamin B group or the vitamin B complex includes, inter alia

Vitamin _{B}1 (thiamin)
Vitamin B₂ (riboflavine)
Vitamin B₃. This designation is frequently used for the compounds nicotinic acid and nicotinamide (niacinamide). Nicotinamide is preferred according to the invention and is preferably contained in the agents according to the invention in quantities of from 0.05 to 1 wt. %, relative to the total agent.
Vitamin B₅ (pantothenic acid, panthenol and pantolactone). In the context of this group, panthenol and/or pantolactone are preferably used. Derivatives of panthenol which may be used according to the invention are in particular the esters and ethers of panthenol and cationically derivatized panthenols. Individual representatives are for example panthenol triacetate, the panthenol monoethyl ether and the monoacetate thereof and the panthenol derivatives disclosed in WO 92/13829. The stated compounds of the vitamin B₅ type are preferably contained in the agents according to the invention in quantities of from 0.05-10 wt. %, relative to the total agent. Quantities of 0.1-5 wt. % are particularly preferred.
Vitamin B₆ (pyridoxine as well as pyridoxamine and pyridoxal).

Vitamin C (ascorbic acid). Vitamin C is preferably used in the agents according to the invention in quantities of from 0.1 to 3 wt. %, relative to the total agent. Use in the form of the palmitic acid ester, the glucosides or phosphates may be preferred. Use in combination with tocopherols may likewise be preferred.

Vitamin E (tocopherols, in particular α-tocopherol). Tocopherol and the derivatives thereof, which include in particular the esters such as the acetate, the nicotinate, the phosphate and the succinate, are preferably contained in the agents according to the invention in quantities of from 0.05-1 wt. %, relative to the total agent.

Vitamin F. The term “vitamin F” is conventionally understood to mean essential fatty acids, in particular linoleic acid, linolenic acid and arachidonic acid.

Vitamin H. Vitamin H denotes the compound (3aS,4S,6aR)-2-oxohexahydrothienol[3,4-d]-imidazole-4-valeric acid, which is now, however, known by the common name biotin. Biotin is contained in the agents according to the invention preferably in quantities of from 0.0001 to 1.0 wt. %, in particular in quantities of from 0.001 to 0.01 wt. %.

The agents according to the invention preferably contain vitamins, provitamins and vitamin precursors from groups A, B, E and H. Panthenol, pantolactone, pyridoxine and the derivatives thereof together with nicotinamide and biotin are particularly preferred.

The compositions according to the invention additionally contain antimicrobial compounds. Suitable antimicrobial compounds are for example cationic surface-active substances, such as for example cetyltrimethylammonium bromide, benzethonium chloride, cetylpyridinium chloride or N,N,N″-tris-(2-hydroxyethyl)-N′-octadecyl-1,3-diaminopropane dihydrofluoride, known as amine fluoride. The antimicrobially active biguanide compounds such as for example polyhexamethylene biguanide (Vantocil® IB, ICI) or 1,1′-hexamethylene-bis-(4-chlorophenyl)-biguanide (“chlorhexidine”) in the form of a water-soluble, compatible salt, for example in the form of the acetate or gluconate, are also well suited. Antimicrobial 5-aminohexahydropyrimidines, for example 1,3-bis-(2-ethylhexyl)-5-methyl-5-aminohexahydropyrimidine (“hexetidine”) are preferably suitable. Further preferably suitable antimicrobial active ingredients are non-cationic, phenolic, antimicrobial substances, in particular halogenated phenols and diphenyl ethers. Particularly suitable antimicrobial compounds of this type are for example 6,6′-methylene-bis-(2-bromo-4-chlorophenol)(“bromochlorophene”) and 2,4,4′-trichloro-2′-hydroxydiphenyl ether (“triclosan”).

Further suitable antimicrobial substances are p-hydroxybenzoic acid esters and sesquiterpene alcohols such as for example bisabolol, farnesol, santalol or nerolidol.

Finally, further synergistic advantages are obtained by using plant extracts (L) in the compositions according to the invention. Therefore, the use of these substances is particularly advantageous.

Such combinations bring about a pleasant fragrance both of the cosmetic composition and of the skin treated therewith and of the treated hair. It is then optionally even possible to dispense with the addition of further perfume oils and fragrances.

Furthermore, this active ingredient according to the invention also has a favorable influence on the moisture balance of the skin and hair. In addition, it displays an antiinflammatory and skin-calming action if for example chamomile or valerian are used. Particularly good effects with regard to the hair are displayed for example by stinging nettle, hops, birch and burdock root.

Conventionally, these extracts are produced by extraction of the entire plant. However, in individual cases it may also be preferable to produce the extracts solely from the blossoms and/or leaves of the plant.

With regard to the plant extracts which may be used according to the invention, reference is made in particular to the extracts which are listed in the table starting on page 44 of the 3rd edition of the “Leiffaden zur Inhaltsstoffdeklaration kosmetischer Mittel” [“Guidelines for the nomenclature of ingredients in cosmetic agents”], published by the German Cosmetic, Toiletry, Perfumery and Detergent Association (IKW), Frankfurt.

According to the invention, preference is above all given to extracts from green tea, oak bark, stinging nettle, witch hazel, hops, henna, chamomile, burdock root, horsetail, hawthorn, lime blossom, almond, aloe vera, pine-needle, horse chestnut, sandalwood, juniper, coconut, mango, apricot, lime, wheat, kiwi fruit, melon, orange, grapefruit, sage, rosemary, birch, mallow, valerian, lady's smock, wild thyme, yarrow, thyme, melissa, restharrow, coltsfoot, marsh mallow, meristem, ginseng, coffee, cocoa, moringa and ginger root.

Particular preference is given to the extracts of green tea, oak bark, stinging nettle, witch hazel, hops, chamomile, burdock root, horsetail, lime blossom, almond, aloe vera, coconut, mango, apricot, lime, wheat, kiwi fruit, melon, orange, grapefruit, sage, rosemary, birch, lady's smock, wild thyme, yarrow, valerian, coffee, cocoa, moringa, restharrow, meristem, ginseng and ginger root.

Extracts which are very particularly suitable for the composition according to the invention are those from green tea, almond, aloe vera, coconut, mango, apricot, lime, wheat, kiwi fruit and melon.

Extracting agents for producing the stated plant extracts may comprise water, alcohols and mixtures thereof. Preferred alcohols are lower alcohols such as ethanol and isopropanol, but in particular polyhydric alcohols such as ethylene glycol and propylene glycol, both as sole extracting agent and in a mixture with water. Plant extracts based on water/propylene glycol in the ratio 1:10 to 10:1 have proven particularly suitable.

The plant extracts may be used according to the invention both in pure and in dilute form. Where used in dilute form, they conventionally contain approx. 2-80 wt. % of active substance and as solvent the extracting agent or extracting agent mixture used to isolate them.

It may furthermore be preferred to use mixtures of a plurality of, in particular of two, different plant extracts in the agents according to the invention.

In addition to plant extracts, a rock crystal extract is has recently also been used in cosmetic compositions. Rock crystal is a modification of silicon dioxide. Silicon dioxide itself is in turn also contained in many different clays and soils as a companion material. For example bentonite contains quartz. Quartz in the form of various silicates is also used for example in homeopathic remedies, for example sodium-aluminum silicate for reducing heartburn or also in Ayurvedic medicine. Sand, which may be contaminated with quartz, is used in cleansing cosmetics as an exfoliant. Quartz also has a mystical significance. Rock crystal is therefore thought of as something special. The varieties of rock crystal, amethyst, smoky quartz, chrysoprase, citrine, morion or rose quartz are in great demand in many cultures as gemstones both for room and clothing decoration. These crystals and minerals are regarded as symbols of beauty, splendor and wealth. Many people have believed and do believe that these crystals have a healing effect, because they are thought to be water that has turned to stone. Other minerals which contain amorphous or very finely divided silicon dioxide are opal and its varieties agate, chalcedony, onyx, cornelian, heliotrope, jasper or flint. Hereinafter, quartz will be taken to mean solely the mineral, crystallized modifications of quartz, which satisfy the structural formula SiO₂ and are free from impurities. Impurities is not intended to mean the traces of other included elements which, for instance, contribute to the color of rose quartz. Under no circumstances is the term “quartz” to be taken to mean silicates, phyllosilicates, talcs, spars etc. In particular, the term “quartz” is understood to mean and covers according to the invention: quartz, tridymite, cristobalite, keatite, coesite, stishovite, rock crystal, smoky quartz, amethyst, chrysoprase, citrine, morion, rose quartz, opal and its varieties agate, chalcedony, onyx, cornelian, heliotrope, jasper or flint. Use is preferably made of quartz, smoky quartz, rock crystal, rose quartz and agate. Smoky quartz, rose quartz and rock crystal are very particularly preferably used. Rock crystal is the most preferred.

Finely ground quartz and an extract of finely ground quartz is used in cosmetic compositions to lend the skin and hair a velvety, soft, pleasant feel. Furthermore, skin and hair gloss is outstandingly increased. However, the skin and hair are not stressed undesirably. Subsequent hair treatments such as cold waving or dyeing are not only not disadvantageously impaired but no impairment of any kind takes place.

The finely ground quartz, or quartz powder, is obtained using conventional methods for comminuting and grinding rock. Quartz powder is used in particular in particle sizes of 0.5 μm to 500 μm. Particular preference is given to particle sizes of 0.5 to 250 μm, very particularly preferably of 10 μm to 200 μm. In a development preferred according to the invention, the finely ground quartz is extracted with the assistance of protic solvents and the resultant quartz extract is used in cosmetic compositions. In this embodiment too, quartz, tridymite, cristobalite, keatite, coesite, stishovite, rock crystal, smoky quartz, amethyst, chrysoprase, citrine, morion, rose quartz, opal and its varieties agate, chalcedony, onyx, cornelian, heliotrope, jasper or flint are used as starting materials for producing a flour and for subsequent extraction to yield “quartz extract”. Use is preferably made of quartz, smoky quartz, rock crystal, rose quartz and agate. Smoky quartz, rose quartz and rock crystal are very particularly preferably used. Rock crystal is the most preferred.

Extracting agents for producing the stated quartz extracts may comprise water, alcohols and mixtures thereof. Water should here be taken to mean both demineralized water and seawater and mineral water. Preferred alcohols are lower alcohols such as ethanol, isopropanol, butanol, iso-butanol, tert.-butanol, pentanols, hexanols or heptanols, and in particular polyhydric alcohols such as glycerols and glycols, in particular glycol, diglycol, glycerol, diglycerol, triglycerol, polyglycerol, ethylene glycol, propylene glycol and butylene glycol, both as sole extracting agent and in a mixture with demineralized water, mineral water or sea water. Extracts based on water and polyhydric alcohols in the ratio 1:50 to 50:1 have proven suitable according to the invention. A ratio of 1:25 to 25:1 is preferred. Particular preference is given to a ratio of 1:10 to 10:1. Very particular preference is given to a ratio of 1:5 to 5:1, wherein a ratio of water to polyhydric alcohol of 3:1 to 1:1 is most preferable. The invention also comprises the teaching that a plurality of alcohols and/or polyhydric alcohols may of course also be used as extracting agents, blended with water. Mineral water is understood to mean water which originates naturally from mineralized sources. They include for example the mineral waters Evian, Spa, L'eau de Vichy etc. The extraction method may comprise all known methods such as for example hot extraction or other methods. Such a quartz extract, once obtained, conventionally contains at least 1 to 100000 ppm of silicon. An extract is preferred which has a minimum quantity of 10 ppm of silicon. Particular preference is given to an extract with a silicon content of at least 50 ppm. Very particular preference is given to an extract with a content of at least 100 ppm. A content of at least 200 ppm of silicon is most preferred. The quantity of silicon in the extract is determined in distilled water by flame spectrometry. The quartz extract may optionally be adjusted with water glass to a constant minimum silicon content. If water glass is used for adjustment of a constant silicon content, it may additionally be necessary to adjust the pH value of the quartz extract. The quartz extract conventionally has a pH value of 4-11, preferably of 6-11, particularly preferably of 7 to 10 and most preferably from 7.5 to 9.5. If adjustment of the pH value of the quartz extract is necessary, adjustment of the pH value is effected using mineral acids such as aqueous solutions of hydrogen halides, sulfuric acid and the salts thereof, sulfurous acid and the salts thereof, phosphorous acid and the salts thereof, phosphoric acid and the salts thereof or with organic acids and the salts thereof such as iminodisuccinic acid, etidronic acid, tartaric acid or citric acid. Adjustment of the pH value of the quartz extract using acids, which also comprise complexing properties, may be preferred. These include, for example, phosphoric acid, iminodisuccinic acid, etidronic acid, tartaric acid or citric acid and the salts thereof. Very particularly preferably, phosphoric acid is used if adjustment of the pH value is needed. One example of a commercially available quartz extract is freely available from Croda under the name Crodarom® Rock Crystal.

It may additionally prove advantageous if penetration aids and/or swelling agents (M) are contained in the compositions according to the invention. These auxiliary substances ensure better penetration of active ingredients into the keratin fiber or help the keratin fiber to swell. Examples of these include urea and urea derivatives, guanidine and the derivatives thereof, arginine and the derivatives thereof, water glass, imidazole and the derivatives thereof, histidine and the derivatives thereof, benzyl alcohol, glycerol, glycol and glycol ethers, propylene glycol and propylene glycol ethers, for example propylene glycol monoethyl ether, carbonates, hydrogencarbonates, diols and triols, and in particular 1,2-diols and 1,3-diols such as for example 1,2-propanediol, 1,2-pentanediol, 1,2-hexanediol, 1,2-dodecanediol, 1,3-propanediol, 1,6-hexanediol, 1,5-pentanediol, 1,4-butanediol.

Finally, experimental results show that the compositions according to the invention are particularly well suited to depositing perfume oils or fragrances on the skin and hair in elevated quantities. At the same time, the perfume oils and fragrances cling to the skin or hair for a markedly longer period. This leads to greater acceptance of such compositions on the part of the consumer. These results are particularly relevant for compositions such as shampoos, shower foams, masks, mask packs, conditioners, leave-on hair masks, styling agents and hair fixing and setting agents.

A further group of very particularly preferred constituents of the compositions according to the invention are perfumes. The outstanding and wholly surprising positive results obtained with compositions containing the active ingredients according to the invention and perfumes have already been described in detail.

The term perfume means perfume oils, fragrances and odoriferous substances. Perfume oils which may be mentioned are mixtures of natural and synthetic odoriferous substances.

Natural odoriferous substances are extracts of blossoms (lily, lavender, rose, jasmine, neroli, ylang-ylang), stems and leaves (geranium, patchouli, petitgrain), fruits (aniseed, coriander, caraway, juniper), fruit peels (bergamot, lemon, orange), roots (mace, angelica, celery, cardamom, costus, iris, calamus), woods (pine, sandalwood, guaiacwood, cedarwood, rosewood), herbs and grasses (tarragon, lemongrass, sage, thyme, chamomile), needles and branches (spruce, fir, pine, mountain pine), resins and balsams (galbanum, elemi, benzoin, myrrh, olibanum, opoponax).

Animal raw materials are also feasible, such as for example civet and castoreum.

Typical synthetic odoriferous substance compounds are products of the ester, ether, aldehyde, ketone, alcohol and hydrocarbon types. Odoriferous substance compounds of the ester type are for example benzyl acetate, phenoxyethyl isobutyrate, p-tert.-butylcyclohexyl acetate, linalyl acetate, dimethylbenzylcarbinyl acetate, phenylethyl acetate, linalyl benzoate, benzyl formate, ethylmethylphenyl glycinate, allylcyclohexyl propionate, styrallyl propionate, cyclohexyl salicylate, floramate, melusate, jasmecyclate and benzyl salicylate. Ethers include, for example, benzyl ethyl ether and ambroxan, the aldehydes for example include linear alkanals with 8-18 carbon atoms, citral, citronellal, citronellyloxyacetaldehyde, cyclamen aldehyde, hydroxycitronellal, lilial and bourgeonal, the ketones include, for example, ionones, α-isomethylionone and methyl cedryl ketone, the alcohols include anethole, citronellol, eugenol, isoeugenol, geraniol, linalool, phenylethyl alcohol and terpineol, the hydrocarbons mainly include terpenes and balsams such as limonene and pinene.

Preferably, however, mixtures of various odoriferous substances are used which together produce an attractive fragrance note. Relatively low volatility essential oils, which are generally used as aroma components, are also suitable as perfume oils, for example sage oil, chamomile oil, clove oil, melissa oil, mint oil, cinnamon leaf oil, lime blossom oil, juniper berry oil, vetiver oil, olibanum oil, galbanum oil, labolanum oil and lavandin oil. Preferably, bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, ambroxan, indole, hedione, sandelice, lemon oil, mandarin oil, orange-blossom oil, orange peel oil, sandalwood oil, neroli oil, allyl amyl glycolate, cyclovertal, lavandin oil, muscatel oil, sage oil, β-damascone, geranium oil Bourbon, cyclohexyl salicylate, Vertofix Coeur, Iso E Super, Fixolide NP, evernyl, iraldein gamma, phenylacetic acid, geranyl acetate, benzyl acetate, rose oxide, romillate, irotyl and floramate are used alone or in mixtures.

Further examples of odoriferous substances which may be in the compositions according to the invention are to be found for example in S. Arctander, Perfume and Flavor Materials, Vols. I and II, Montclair, N.J., 1969, self-publication or K. Bauer, D. Garbe und H. Surburg, Common Fragrance and Flavor Materials, 3th ed., Wiley-VCH, Weinheim 1997.

If it is to be perceptible, an odoriferous substance must be volatile, wherein, in addition to the nature of the functional groups and the structure of the chemical compound, an important role is also played by molar mass. Most odoriferous substances accordingly have molar masses of up to approx. 200 Dalton, while molar masses of 300 Dalton and above tend to be the exception. Due to the differing volatility of odoriferous substances, the odor of a perfume or fragrance composed of two or more odoriferous substances varies over the course of vaporization, it being possible to subdivide odor impressions into “head or top note”, “heart or middle note” and “end note or dry-out”. Since odor perception largely also depends on odor intensity, the head note of a perfume or fragrance does not solely consist of highly volatile compounds, while the end note largely consists of less volatile, i.e. tenacious odoriferous substances.

Tenacious odoriferous substances which may be used for the purposes of the present invention are, for example, essential oils such as angelica root oil, anise oil, arnica blossom oil, basil oil, bay oil, bergamot oil, champak flower oil, silver fir oil, silver fir cone oil, elemi oil, eucalyptus oil, fennel oil, spruce oil, galbanum oil, geranium oil, ginger grass oil, guaiacwood oil, gurjun balsam oil, helichrysum oil, ho oil, ginger oil, iris oil, cajeput oil, calamus oil, chamomile oil, camphor oil, canaga oil, cardamom oil, cassia oil, Scotch fir oil, copaiba balsam oil, coriander oil, spearmint oil, caraway oil, cumin oil, lavender oil, lemongrass oil, lime oil, mandarin oil, melissa oil, ambrette oil, myrrh oil, clove oil, neroli oil, niaouli oil, olibanum oil, orange oil, origanum oil, palmarosa oil, patchouli oil, Peru balsam oil, petitgrain oil, pepper oil, peppermint oil, pimento oil, pine oil, rose oil, rosemary oil, sandalwood oil, celery oil, spike oil, star anise oil, terpentine oil, thuja oil, thyme oil, verbena oil, vetiver oil, juniper berry oil, wormwood oil, wintergreen oil, ylang-ylang oil, hyssop oil, cinnamon oil, cinnamon leaf oil, citronellol, lemon oil and cypress oil.

Higher-boiling or solid odoriferous substances of natural or synthetic origin may, however, also be used advantageously for the purposes of the present invention as tenacious odoriferous substances or odoriferous substance mixtures, i.e. fragrances. These compounds include the compounds stated below and mixtures thereof: ambrettolide, -amylcinnamaldehyde, anethole, anisaldehyde, anisyl alcohol, anisole, methyl anthranilate, acetophenone, benzyl acetone, benzaldehyde, benzoic acid ethyl ester, benzophenone, benzyl alcohol, benzyl acetate, benzyl benzoate, benzyl formate, benzyl valerate, borneol, bornyl acetate, -bromostyrene, n-decylaldehyde, n-dodecylaldehyde, eugenol, eugenol methyl ether, eucalyptol, farnesol, fenchone, fenchyl acetate, geranyl acetate, geranyl formate, heliotropin, methyl heptine carbonate, heptaldehyde, hydroquinone dimethyl ether, hydroxycinnamaldehyde, hydroxycinnamyl alcohol, indole, irone, isoeugenol, isoeugenol methyl ether, isosafrole, jasmone, camphor, carvacrol, carvone, p-cresol methyl ether, coumarin, p-methoxyacetophenone, methyl N-amyl ketone, methylanthranilic acid methyl ester, p-methylacetophenone, methylchavicol, p-methylquinoline, methyl naphthyl ketone, methyl n-nonylacetaldehyde, methyl n-nonyl ketone, muscone, -naphthol ethyl ether, -naphthol methyl ether, nerol, nitrobenzene, n-nonylaldehyde, nonyl alcohol, n-octylaldehyde, p-oxyacetophenone, pentadecanolide, -phenyl ethyl alcohol, phenylacetaldehyde dimethyl acetal, phenylacetic acid, pulegone, safrole, isoamyl salicylate, methyl salicylate, hexyl salicylate, cyclohexyl salicylate, santalol, skatole, terpineol, thymene, thymol, -undelactone, vanillin, veratrumaldehyde, cinnamaldehyde, cinnamyl alcohol, cinnamic acid, ethyl cinnamate, benzyl cinnamate.

More highly volatile odoriferous substances, which may advantageously be used for the purposes of the present invention, include in particular lower-boiling odoriferous substances of natural or synthetic origin, which may be used alone or in mixtures. Examples of more highly volatile odoriferous substances are alkyl isothiocyanates (alkyl mustard oils), butanedione, limonene, linalool, linalyl acetate and propionate, menthol, menthone, methyl-n-heptenone, phellandrene, phenylacetaldehyde, terpinyl acetate, citral, citronellal.

All the above-stated odoriferous substances may be used alone or in mixture according to the present invention with the above-stated advantages.

If the boiling points of the individual fragrances lie substantially below 300° C., a preferred embodiment of the invention is provided, wherein preferably at least 50% of the fragrances contained therein have a boiling point of below 300° C., advantageously at least 60%, more advantageously at least 70%, even more advantageously at least 80%, extremely advantageously at least 90%, in particular even 100%.

Boiling points of below 300° C. are advantageous because the fragrances in question would not be sufficiently volatile at higher boiling points. However, a certain fragrance volatility is advantageous if the fragrance is at least in part to “flow out” of the particle and have its fragrancing effect.

It has already been observed above that some unstable perfume constituents are sometimes not very compatible with the carrier material and break down at least in part after incorporation into the carrier, in particular if the carrier is porous mineral carrier, such as for example clay, or zeolite, above all dehydrated and/or activated zeolite. Unstable fragrances for the purposes of the present invention may be identified in that a perfume composition comprising at least 6 fragrances is incorporated into activated/dehydrated zeolite X and the resultant sample is stored for 24 hours at room temperature. Then the fragrances are extracted with acetone and analyzed by gas chromatography, to determine stability. A fragrance is regarded as unstable for the purposes of the present invention if at least 50 wt. %, preferably at least 65 wt. %, advantageously at least 80 wt. %, in particular at least 95 wt. % of this fragrance has broken down into breakdown products and cannot be produced again on extraction.

If less than 15 wt. %, preferably less than 8 wt. %, advantageously less than 6 wt. %, still more advantageously less than 3 wt. % of unstable perfume is contained in the agent according to the invention, relative to the total quantity of perfume ad-/absorbed in/on the particles, a preferred embodiment of the invention is provided, wherein the unstable perfume in particular comprises the group allyl alcohol esters, esters of secondary alcohols, esters of tertiary alcohols, allylic ketones, condensation products of amines and aldehydes, acetals, ketals and mixtures of the above.

If the perfume ad-/absorbed in/on the particles contains at least 4, advantageously at least 5, more advantageously at least 6, even more advantageously at least 7, yet more advantageously at least 8, preferably at least 9, in particular at least 10 different odoriferous substances, a preferred embodiment of the invention is provided.

If the log P value of the perfume components ad-/absorbed in/on the particles is substantially at least 2, preferably at least 3 or more, such that thus at least 40%, advantageously at least 50%, more advantageously at least 60%, even more advantageously at least 70%, preferably at least 80%, in particular 90% of the perfume components satisfy this log requirement, a preferred embodiment of the invention is provided.

The log P value is a measure of the hydrophobicity of the perfume components. It is the base-ten logarithm of the distribution coefficient between n-octanol and water. The octanol/water distribution coefficient of a perfume constituent is the ratio between its equilibrium concentrations in water and octanol. A perfume constituent with a higher distribution coefficient P is more strongly hydrophobic. The stated conditions for log P are therefore advantageous, because it is thereby ensured that the fragrances may be better retained in the pores of the carrier and are better deposited on objects which are treated with the particles (for example indirectly by treatment with a detergent formulation containing the particles according to the invention). The log P value of many perfume constituents is stated in the literature; for example the Pomona 92 database, obtainable from Daylight Chemical Information Systems, Inc., (Daylog CIS), Irvine, Calif., contains many such values together with references to the original literature. Log P values may also be calculated, for example using the “C LOG P” program published by Daylight CIS, as stated above. Calculated log P values are generally called C log P values. For the purposes of the present invention, the term log P values also covers C log−P values. Preferably, C log−P values should be used for estimating hydrophobicity when no experimental log P values are available for specific perfume constituents.

If desired, the perfume may also be combined with a perfume fixative. It is assumed that perfume fixatives are capable of slowing down emission of the more highly volatile fractions of perfume.

According to a further preferred embodiment, the perfume ab-/adsorbed in/on the carrier comprises a perfume fixative, preferably in the form of diethyl phthalates, musk(derivatives) and mixtures thereof, the quantity of fixative amounting to preferably 1 to 55 wt. %, advantageously 2 to 50 wt. %, more advantageously 10 to 45 wt. %, in particular 20 to 40 wt. % of the total quantity of perfume.

According to a further preferred embodiment, the particles contain an agent which increases the viscosity of liquids, in particular of perfume, preferably PEG (polyethylene glycol), advantageously with a molecular weight of 400 to 2000, the viscosity-increasing agent preferably being contained in quantities of from 0.1 to 20 wt. %, advantageously 0.15 to 10 wt. %, more advantageously 0.2 to 5 wt. %, in particular 0.25 to 3 wt. %, relative to the particles.

It has been found that agents which increase the viscosity of liquids, in particular of perfume, make a further contribution to stabilization of the perfume in the particles, if a nonionic surfactant is present at the same time.

Viscosity-increasing agents are preferably polyethylene glycols (PEG for short), which may be described by the general formula I:

H—(O—CH₂—CH₂)ₙ—OH (I),

in which the degree of polymerization n may vary from approx. 5 to >100000, corresponding to molar masses of 200 to 5000000 gmol⁻¹. Products with molar masses of below 25,000 g/mol are known as polyethylene glycols proper, while high molecular weight products are often described in the literature as polyethylene oxides (PEOX for short). The polyethylene glycols preferably used may have a linear or branched structure, linear polyethylene glycols being particularly preferred, and be end group-terminated. Particularly preferred polyethylene glycols include those with relative molecular masses of between 400 and 2000. Polyethylene glycols may also in particular be used which are per se in the liquid state at room temperature and a pressure of 1 bar; these are above all polyethylene glycols with relative molecular masses of 200, 400 and 600. The perfumes are added to the total composition in general in a quantity of 0.05 to 5 wt. %, preferably of 0.1 to 2.5 wt. %, particularly preferably of 0.2 to 1.5 wt. %, relative to the total composition.

The perfumes may be added to the compositions for perfuming purposes in liquid form, undiluted or diluted with a solvent. Solvents suitable for this purpose are for example ethanol, isopropanol, diethylene glycol monoethyl ether, glycerol, propylene glycol, 1,2-butylene glycol, dipropylene glycol, diethyl phthalate, triethyl citrate, isopropyl myristate etc.

Moreover, the perfumes for the compositions according to the invention may be adsorbed on a carrier, which ensures both fine distribution of the odoriferous substances in the product and controlled release during use. Such carriers may be porous inorganic materials such as sodium sulfate, silica gels, zeolites, gypsums, clays, clay granules, aerated concrete etc. or organic materials such as woods, and cellulose-based substances.

The perfume oils for the compositions according to the invention may also be microencapsulated, spray-dried or present as inclusion complexes or extrusion products and added in this form to the compositions to be perfumed.

Optionally, the properties of the perfume oils modified in this way may be further optimized with regard to more targeted fragrance release by “coating” with suitable materials, for which purpose waxy plastics such as for example polyvinyl alcohol are preferably used.

The customer's perception of the cosmetic compositions, in particular due to aesthetically pleasing packaging, optionally in conjunction with aromatic fragrance notes, may lead him/her to associate the composition according to the invention with products consumed for pleasure, such as for example confectionery or beverages. As a result of this association it is not in principle possible to rule out, in particular in the case of children, oral intake or swallowing of the cosmetic composition. In a preferred embodiment, the compositions according to the invention therefore contain a bitter substance, in order to prevent swallowing or accidental ingestion. For this purpose, bitter substances are preferred according to the invention which are soluble in water at 20° C. at a rate of at least 5 g/l.

From the point of view of undesired interaction with fragrance components optionally contained in the cosmetic compositions, in particular of modification of the fragrance note perceived by the consumer, ionogenic bitter substances have proven superior to non-ionogenic bitter substances, and ionogenic bitter substances, preferably consisting of organic cation(s) and organic anion(s), are therefore preferred for the preparations according to the invention.

Substances which are outstandingly suitable as bitter substances according to the invention are quaternary ammonium compounds, which contain an aromatic group both in the cation and in the anion. Such a compound is benzyldiethyl((2,6-xylylcarbamoyl)methyl)ammonium benzoate, which is commercially available for example under the tradenames Bitrex® and Indigestin®. This compound is also known by the name Denatonium Benzoate.

The bitter substance is contained in the compositions according to the invention in quantities of from 0.0005 to 0.1 wt. %, relative to the shaped article. Quantities of from 0.001 to 0.05 wt. % are particularly preferred.

For the purposes of the invention, short-chain carboxylic acids (N) may advantageously be used as a constituent in the compositions. For the purposes of the invention, short-chain carboxylic acids and the derivatives thereof are understood to mean carboxylic acids which may be saturated or unsaturated and/or linear or branched or cyclic and/or aromatic and/or heterocyclic and have a molecular weight of less than 750. Preference may be given, for the purposes of the invention, to saturated or unsaturated straight-chain or branched carboxylic acids with a chain length of from 1 to 16 C atoms in the chain, very particular preference being given to those with a chain length of from 1 to 12 C atoms.

One use for short-chain carboxylic acids is to adjust the pH value of the cosmetic compositions according to the invention. The active ingredient complex (A) according to the invention results, together with a short-chain carboxylic acid, in improved skin smoothness and in improved skin structure and smoothed hair structure.

In addition to the short-chain carboxylic acids according to the invention listed above by way of example themselves, the physiologically acceptable salts thereof may also be used according to the invention. Examples of such salts are the alkali metal, alkaline earth metal, zinc salts and ammonium salts, these being understood within the bounds of present application also to mean the mono-, di- and trimethyl-, -ethyl- and -hydroxyethylammonium salts. In addition, however, neutralized acids may also be used with alkaline-reacting amino acids, such as for example arginine, lysine, ornithine and histidine. The sodium, potassium, ammonium and arginine salts are preferred salts. It may furthermore be preferred for formulation reasons to select the carboxylic acids as active ingredient from among the water-soluble representatives, in particular the water-soluble salts.

The short-chain carboxylic acids very particularly preferred according to the invention include hydroxycarboxylic acids and in turn in particular the dihydroxy-, trihydroxy- and polyhydroxycarboxylic acids and the dihydroxy-, trihydroxy- and polyhydroxy-di-, tri- and polycarboxylic acids.

Examples of particularly suitable hydroxycarboxylic acids are glycolic acid, glyceric acid, lactic acid, malic acid, tartaric acid or citric acid. It goes without saying that the teaching according to the invention also includes these acids in the form of mixed salts for example with amino acids. This may be preferred according to the invention. Examples of amino acids which may be used as mixed salts with these hydroxycarboxylic acids are carnitine, taurine, histidine, lysine, arginine and ornithine. A typical representative of mixed salts according to the invention is for example carnitine tartrate.

It goes without saying that the teaching according to the invention comprises all isomeric forms, such as cis-trans isomers, diastereomers and chiral isomers.

According to the invention, it is also possible to use a mixture of a plurality of active ingredients from this group.

The short-chain carboxylic acids for the purposes of the invention may comprise one, two, three or more carboxy groups. Carboxylic acids having a plurality of carboxy groups, in particular di- and tricarboxylic acids, are preferred for the purposes of the invention. The carboxy groups may be present entirely or in part as esters, acid anhydride, lactone, amide, imidic acid, lactam, lactim, dicarboximide, carbohydrazide, hydrazone, hydroxam, hydroxime, amidine, amide oxime, nitrile, phosphonate or phosphate esters. It goes without saying that the carboxylic acids according to the invention may be substituted along the carbon chain or the ring skeleton. The substituents of carboxylic acids according to the invention include for example C₁-C₈ alkyl, C₂-C₈ alkenyl, aryl, aralkyl and aralkenyl, hydroxymethyl, C₂-C₈ hydroxyalkyl, C₂-C₈ hydroxyalkenyl, aminomethyl, C₂-C₈ aminoalkyl, cyano, formyl, oxo, thioxo, hydroxy, mercapto, amino, carboxy or imino groups. Preferred substituents are C₁-C₈ alkyl, hydroxymethyl, hydroxy, amino and carboxy groups. Substituents in [sic] position are very particularly preferred. Very particularly preferred substituents are hydroxy, alkoxy and amino groups, the amino function optionally being further substituted by alkyl, aryl, aralkyl and/or alkenyl residues. Phosphonate and phosphate esters are furthermore likewise preferred carboxylic acid derivatives.

Examples of carboxylic acids according to the invention which may be mentioned are formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphoric acid, benzoic acid, o,m,p-phthalic acid, naphthoic acid, toluic acid, hydratropic acid, atropic acid, cinnamic acid, isonicotinic acid, nicotinic acid, bicarbamic acid, 4,4′-dicyano-6,6′-binicotinic acid, 8-carbamoyloctanoic acid, 1,2,4-pentanetricarboxylic acid, 2-pyrrolecarboxylic acid, 1,2,4,6,7-naphthalenepentaacetic acid, malonaldehydic acid, 4-hydroxyphthalamidic acid, 1-pyrazolecarboxyilic acid, gallic acid or propanetricarboxylic acid, a dicarboxylic acid selected from the group which is formed by compounds of the general formula (N-I),

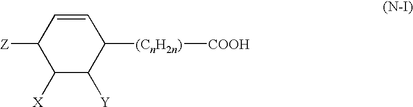

in which Z denotes a linear or branched, alkyl or alkenyl group with 4 to 12 carbon atoms, n denotes a number from 4 to 12 and one of the two groups X and Y denotes a COOH group and the other denotes hydrogen or a methyl or ethyl residue, dicarboxylic acids of the general formula (N-I) which additionally also bear 1 to 3 methyl or ethyl substituents on the cyclohexene ring and dicarboxylic acids formally arising from the dicarboxylic acids according to the formula (N-I) by attachment of a molecule of water onto the double bond in the cyclohexene ring.

Dicarboxylic acids of the formula (N-I) are known in the literature.

A production method is revealed for example by U.S. Pat. No. 3,753,968. German patent 22 50 055 discloses the use of these dicarboxylic acids in liquid soap compositions. German published patent application 28 33 291 discloses deodorizing agents which contain zinc or magnesium salts of these dicarboxylic acids. Finally, German published patent application 35 03 618 discloses agents for washing and rinsing hair in which, through the addition of these dicarboxylic acids, a markedly improved hair cosmetic action of the water-soluble ionic polymers contained in the agent is obtained. Finally, agents for hair treatment which have hair care effects are known from German published patent application 197 54 053.

The dicarboxylic acids of the formula (N-I) may be produced, for example, by reacting polyunsaturated dicarboxylic acids with unsaturated monocarboxylic acids in the manner of a Diels-Alder cyclization. A polyunsaturated fatty acid will conventionally be used as starting material for a dicarboxylic acid component. Linoleic acid, which is obtainable from natural fats and oils, is preferred. Acrylic acid, but also for example methacrylic acid and crotonic acid, are in particular preferred as the monocarboxylic acid component. Diels-Alder reactions conventionally give rise to isomer mixtures in which one component is present in excess. These isomer mixtures may likewise be used according to the invention in the same manner as the pure compounds.

In addition to the preferred dicarboxylic acids according to the formula (N-I), it is also possible according to the invention to use those dicarboxylic acids which differ from the compounds according to the formula (N-I) by 1 to 3 methyl or ethyl substituents on the cyclohexyl ring or are formally formed from these compounds by attachment of a molecule of water onto the double bond of the cyclohexene ring.

The dicarboxylic acid (mixture) obtained by reacting linoleic acid with acrylic acid has proved particularly active according to the invention. This is a mixture of 5- and 6-carboxy-4-hexyl-2-cyclohexene-1-octanoic acid. Such compounds are commercially available under the names Westvaco Diacid® 1550 and Westvaco Diacid® 1595 (manufacturer: Westvaco).

In addition to the short-chain carboxylic acids according to the invention listed above by way of example themselves, the physiologically acceptable salts thereof may also be used according to the invention. Examples of such salts are the alkali metal, alkaline earth metal, zinc salts and ammonium salts, these being understood within the bounds of present application also to mean the mono-, di- and trimethyl-, -ethyl- and -hydroxyethylammonium salts. However, for the purposes of the invention, neutralized acids may very particularly preferably be used with alkaline-reacting amino acids, such as for example arginine, lysine, ornithine and histidine. It may furthermore be preferred for formulation reasons to select the carboxylic acids from among the water-soluble representatives, in particular the water-soluble salts.

It is furthermore preferred according to the invention to use hydroxycarboxylic acids and in turn in particular the dihydroxy-, trihydroxy- and polyhydroxycarboxylic acids and the dihydroxy-, trihydroxy- and polyhydroxy- di-, tri- and polycarboxylic acids jointly in the agents. It has here been found that, in addition to hydroxycarboxylic acids, the hydroxycarboxylic acid esters and mixtures of hydroxycarboxylic acids and the esters thereof as well as polymeric hydroxycarboxylic acids and the esters thereof may be very particularly preferred. Preferred hydroxycarboxylic acid esters are for example full esters of glycolic acid, lactic acid, malic acid, tartaric acid or citric acid. Further hydroxycarboxylic acid esters which are suitable in principle are esters of β-hydroxypropionic acid, of tartronic acid, of D-gluconic acid, of saccharic acid, of mucic acid or of glucuronic acid. Suitable alcohol components of these esters are primary, linear or branched aliphatic alcohols having 8-22 C atoms, thus for example fatty alcohols or synthetic fatty alcohols. The esters of C₁₂-C₁₅ fatty alcohols are here particularly preferred. Esters of this type are commercially available, for example under the trademark Cosmacol® from EniChem, Augusta Industriale. Particularly preferred polyhydroxypolycarboxylic acids are polylactic acid and polytartaric acid and the esters thereof.

According to the invention it is very particularly preferred to use so-called edible acids as short-chain carboxylic acids for the purposes of the invention.

These active ingredients according to the invention are present in the agents for example in concentrations of 0.01 wt. % up to 20 wt. %, preferably of 0.05 wt. % up to 15 wt. % and very particularly preferably in quantities of 0.1 wt. % up to 5 wt. %.

Polyhydroxy compounds are a very particularly varied and interesting group of cosmetic active ingredients. The use according to the invention of polyhydroxy compounds as active ingredient with the other components according to the invention may therefore be particularly preferred. Polyhydroxy compounds for the purposes of the invention are understood to mean all substances which satisfy the definition in Römpp's Lexikon der Chemie [Römpp lexicon of chemistry], 1999 edition, Verlag Georg Thieme. Accordingly, polyhydroxy compounds are understood to mean organic compounds with at least two hydroxy groups.

For the purposes of the present invention these include in particular: polyols with at least two hydroxy groups, such as for example trimethylolpropane, ethoxylates and/or propoxylates with 1 to 50 mol of ethylene oxide and or propylene oxide of the above-stated polyols, carbohydrates, sugar alcohols and sugar and the salts thereof,

in particular monosaccharides, disaccharides, trisaccharides and oligosaccharides, these possibly also being present in the form of aldoses, ketoses and/or lactoses and protected by conventional —OH and —NH protective groups known from the literature, such as for example the triflate group, the trimethylsilyl group or acyl groups and furthermore in the form of methyl ethers and as phosphate esters,
amino deoxy sugar, deoxy sugar, thio sugar, these possibly also being present in the form of aldoses, ketoses and/or lactoses and protected by conventional —OH and —NH protective groups known from the literature, such as for example the triflate group, the trimethylsilyl group or acyl groups and furthermore in the form of methyl ethers and as phosphate esters.

Of these, very particular preference is given to monosaccharides with 3 to 8 C atoms, such as for example trioses, tetroses, pentoses, hexoses, heptoses and octoses, these possibly also being present in the form of aldoses, ketoses and/or lactoses and protected by conventional —OH and —NH protective groups known from the literature, such as for example the triflate group, the trimethylsilyl group or acyl groups and furthermore in the form of methyl ethers and as phosphate esters.

Oligosaccharides with up to 50 monomer units are furthermore preferred, these possibly also being present in the form of aldoses, ketoses and/or lactoses and protected by conventional —OH and —NH protective groups known from the literature, such as for example the triflate group, the trimethylsilyl group or acyl groups and furthermore in the form of methyl ethers and as phosphate esters.

Examples of polyols according to the invention which may be mentioned are sorbitol, inositol, mannitol, tetritols, pentitols, hexitols, threitol, erythritol, adonitol, arabitol, xylitol, dulcitol, erythrose, threose, arabinose, ribose, xylose, lyxose, glucose, galactose, mannose, allose, altrose, gulose, idose, talose, fructose, sorbose, psicose, tegatose, deoxyribose, glucosamine, galactosamine, rhamnose, digitoxose, thioglucose, sucrose, lactose, trehalose, maltose, cellobiose, melibiose, gentiobiose, rutinose, raffinose and cellotriose. Reference may furthermore be made to the relevant specialist literature such as for example Beyer-Walter, Lehrbuch der organischen Chemie [textbook of organic chemistry], S. Hirzel Verlag Stuttgart, 19th edition, section III, pages 393 et seq.

Preferred polyhydroxy compounds are sorbitol, inositol, mannitol, threitol, erythreitol, erythrose, threose, arabinose, ribose, xylose, glucose, galactose, mannose, allose, fructose, sorbose, deoxyribose, glucosamine, galactosamine, sucrose, lactose, trehalose, maltose and cellobiose. Glucose, galactose, mannose, fructose, deoxyribose, glucosamine, sucrose, lactose, maltose and cellobiose are particularly preferably used. It is, however, very particularly preferred to use glucose, galactose, mannose, fructose, sucrose, lactose, maltose or cellobiose.

In one particularly preferred embodiment, at least one polyhydroxy compound with at least 2 OH groups is present as active ingredient. Of these compounds, those having 2 to 12 OH groups and in particular those having 2, 3, 4, 5, 6 or 10 OH groups are preferred.

Polyhydroxy compounds with 2 OH groups are for example glycol (CH₂(OH)CH₂OH) and other 1,2-diols such as H—(CH₂)ₙ—CH(OH)CH₂OH with n=1, 2, 3, 4, 5, 6, 7, 8, 9, 10, 11, 12, 13, 14, 15, 16, 17, 18, 19, 20. 1,3-Diols such as H—(CH₂)ᵣ—CH(OH)CH₂CH₂OH with n=1, 2, 3, 4, 5, 6, 7, 8, 9, 10, 11, 12, 13, 14, 15, 16, 17, 18, 19, 20 may also be used according to the invention. (m,n+1)- or (m,n+2)-diols having non-terminal OH groups may likewise be used.

Important representatives of polyhydroxy compounds with 2 OH groups are also polyethylene and polypropylene glycols.

Among polyhydroxy compounds with 3 OH groups, glycerol is of great significance.

To summarize, preferred compositions according to the invention are those in which the polyhydroxy compound is selected from ethylene glycol, propylene glycol, polyethylene glycol, polypropylene glycol, glycerol, glucose, fructose, pentaerythritol, sorbitol, mannitol, xylitol and the mixtures thereof.

Irrespective of the type of polyhydroxy compound with at least 2 OH groups which is used, preferred agents according to the invention are those which, relative to the weight of the agent, contain 0.01 to 5 wt. %, preferably 0.05 to 4 wt. %, particularly preferably 0.05 to 3.5 wt. % and in particular 0.1 to 2.5 wt. % of polyhydroxy compound(s).

The agents according to the invention may additionally particularly preferentially contain polyethylene glycol ethers of the formula (IV)

H(CH₂)ₖ(OCH₂CH₂)ₙOH (IV)

in which k means a number between 1 and 18, with the values 0, 10, 12, 16 and 18 being particularly preferred, and n means a number between 2 and 20, with the values 2, 4, 5, 6, 7, 8, 9, 10, 12 and 14 being particularly preferred. Preferred among these are the alkyl derivatives of diethylene glycol, triethylene glycol, tetraethylene glycol, pentathylene glycol, hexaethylene glycol, heptaethylene glycol, octaethylene glycol, nonaethylene glycol, decaethylene glycol, dodecaethylene glycol and tetradecaethylene glycol and the alkyl derivatives of dipropylene glycol, tripropylene glycol, tetrapropylene glycol, pentapropylene glycol, hexapropylene glycol, heptapropylene glycol, octapropylene glycol, nonapropylene glycol, decapropylene glycol, dodecapropylene glycol and tetradecapropylene glycol, with methyl, ethyl, propyl, n-butyl, n-pentyl, n-hexyl, n-heptyl, n-octyl, n-nonyl, n-decyl, n-undecyl, n-dodecyl and n-tetradecyl derivatives being preferred.

It has been found that mixtures of “short-chain” polyalkylene glycol ethers with such “long-chain” polyalkylene glycol ethers have advantages. “Short or long-chain” relates in this connection to the degree of polymerization of the polyalkylene glycol.

Mixtures of polyalkylene glycol ethers with a degree of oligomerization of 5 or less with polyalkylene glycol ethers with a degree of oligomerization of 7 or more are particularly preferred. Preferred mixtures are those of alkyl derivatives of diethylene glycol, triethylene glycol, tetraethylene glycol, pentaethylene glycol, dipropylene glycol, tripropylene glycol, tetrapropylene glycol or pentapropylene glycol with alkyl derivatives of hexaethylene glycol, heptaethylene glycol, octaethylene glycol, nonaethylene glycol, decaethylene glycol, dodecaethylene glycol, hexapropylene glycol, heptapropylene glycol, octapropylene glycol, nonapropylene glycol, decapropylene glycol, dodecapropylene glycol or tetradecapropylene glycol, with in both cases the n-octyl, n-decyl, n-dodecyl and n-tetradecyl derivatives being preferred.

Particularly preferred agents according to the invention are characterized in that they contain at least one polyalkylene glycol ether (IVa) of the formula (IV), in which n denotes the numbers 2, 3, 4 or 5 and at least one polyalkylene glycol ether (IVb) of the formula (IV), in which n denotes the numbers 10, 12, 14 or 16, the weight ratio of (IVb) to (IVa) amounting to 10:1 to 1:10, preferably 7.5:1 to 1:5 and in particular 5:1 to 1:1.

Very particularly preferred polyols of the present invention are polyols with 2 to 12 C atoms in the molecular framework. These polyols may be straight-chain, branched, cyclic and/or unsaturated. The hydroxy groups are here very particularly preferably terminally adjacent or separated terminally by the remainder of the chain. Examples of these polyols which may be mentioned are: glycol, polyethylene glycol up to a molecular weight of up to 1000 dalton, neopentyl glycol, partial glycerol ethers with a molecular weight of up to 1000 dalton, 1,2-propanediol, 1,3-propanediol, glycerol, 1,2-butanediol, 1,3-butanediol, 1,4-butanediol, 1,2,3-butanetriol, 1,2,4-butanetriol, pentanediols, for example 1,2-pentanediol, 1,5-pentanediol, hexanediols, 1,2-hexanediol, 1,6-hexanediol, 1,2,6-hexanetriol, 1,4-cyclohexanediol, 1,2-cyclohexanediol, heptanediols, 1,2-heptanediol, 1,7-heptanediol, octanediols, 1,2-octanediol, 1,8-octanediol, 2-ethyl-1,3-hexanediol, octadienols, decadienols, dodecanediols, 1,2-dodecanediol, 1,12-dodecanediol, 1,12-dodecanediol with 10 mol EO, dodecadienols.

It goes without saying that the teaching according to the invention comprises all isomeric forms, such as cis-trans isomers, diastereomers, epimers, anomers and chiral isomers.

According to the invention, it is also possible to use a mixture of a plurality of polyhydroxy compounds.

The polyhydroxy compounds according to the invention are present in the compositions in concentrations of 0.01 wt. % to 20 wt. %, preferably of 0.05 wt. % to 15 wt. % and very particularly preferably in quantities of 0.1 wt. % to 10 wt. %.

Further optional constituents which may commonly be used in cosmetic compositions are preservatives. The substances listed in Appendix 6, parts A and B of the European Cosmetics Directive are used as preservatives. Mild preservation, ideally without the addition of typical preservatives, is particularly preferred. The following substances and mixtures thereof are generally used:

aromatic alcohols, such as for example phenoxyethanol, benzyl alcohol, phenethyl alcohol, phenoxyisopropanol,
aldehydes such as for example formaldehyde solution and paraformaldehyde, glutaraldehyde, parabens, for example methylparaben, ethylparaben, propylparaben, butylparaben, isobutylparaben
1,2-alkanediols with 5 to 22 carbon atoms in the carbon chain, such as for example 1,2-pentanediol, 1,2-hexanediol, 1,2-heptanediol, 1,2-decanediol, 1,2-dodecanediol, 1,2-hexadecanediol,
formaldehyde-releasing compounds, such as for example DMDM hydantoin, diazolidinyl urea
halogenated compounds such as for example isothiazolinones, such as for example methylchloroisothiazolinone/methylisothiazolinones, triclosan, triclocarban, iodopropynyl butyl carbamate, 5-bromo-5-nitro-1,3-dioxane, chlorhexidine digluconate and chlorhexidine acetate, 2-bromo-2-nitropropane-1,3-diol, methyldibromoglutaronitrile,
inorganic compounds such as for example sulfites, boric acid and borates, bisulfites,
cationic substances such as for example Quaternium-15, benzalkonium chloride, benzethonium chloride, polyaminopropyl biguanide,
organic acids and the physiologically acceptable salts thereof such as for example citric acid, lactic acid, acetic acid, benzoic acid, sorbic acid, salicylic acid, dehydroacetic acid
active ingredients with additional actions such as for example zinc pyrithione, piroctone olamine,
antioxidants such as for example BHT (butylated hydroxytoluene), BHA (butylated hydroxyanisole), propyl gallate, t-butylhydroquinone,
complexing agents such as for example EDTA and the derivatives thereof, HEDTA and the derivatives thereof, etidronic acid and the salts thereof,
as well as mixtures of the above-listed substances.

In a further particularly preferred type of the compositions according to the invention, it is also possible to reduce water activity in the compositions according to the invention to such an extent that growth of microorganisms can no longer proceed. Glycerol and sorbitol are in particular used for this purpose.

The compositions according to the invention also contribute to ensuring excellent preservation with the mild preservative additives. Completely dispensing with preservatives is, however, also possible and preferred according to the invention.

The quantities of preservative amount to 0 to 5 wt. %, preferably from 0-2 wt. %, particularly preferably from 0-1 wt. % and very particularly preferably from 0 to 0.8 wt. % relative to the total quantity of the composition.

Further optional constituents of the compositions according to the invention are deodorant active ingredients. Deodorant active ingredients may not only be used in deodorants to prevent armpit perspiration. They may also be used in skin care agents in order to have an influence on perspiration in other skin locations. This also includes, for example, the scalp.

The compositions according to the invention bring about a distinctly analytically detectable increase in the deposition of deodorizing active substances on skin and hair. In panel testing, this is inter alia also perceptible thanks to a distinctly extended duration of action.

Esterase inhibitors may be added as deodorant active ingredients. These preferably comprise trialkyl citrates such as trimethyl citrate, tripropyl citrate, triisopropyl citrate, tributyl citrate and in particular triethyl citrate (Hydagen® CAT, Cognis). These substances inhibit enzyme activity and so reduce odor formation. Cleavage of the citric acid ester probably here results in release of the free acid which reduces the pH value on the skin to such an extent that the enzymes are inhibited thereby. Further substances which may be considered as esterase inhibitors are dicarboxylic acids and the esters thereof, such as for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and the esters thereof such as for example citric acid, malic acid, tartaric acid or tartaric acid diethyl ester. Antibacterial active ingredients which have an influence on microbial flora and kill or inhibit the growth of perspiration-decomposing bacteria may likewise be present in stick preparations. Examples of these are chitosan, phenoxyethanol and chlorhexidine gluconate. 5-Chloro-2-(2,4-dichlorophenoxy)-phenol, which is distributed by Ciba-Geigy, Basel (CH) under the trademark Irgasan®, has also proved particularly effective.

The active ingredient complex according to the invention may furthermore be used in dyeing agents. These dyeing agents naturally also contain dye precursors as further constituents.

Further very particularly preferred optional constituents of the compositions are dye precursors. Dye precursors are oxidation dye precursors of the developer (X1) and coupler type (X2), natural and synthetic direct dyes (Y) and precursors of nature-analogous dyes, such as indole and indoline derivatives, and mixtures of representatives of one or more of these groups.

Oxidation dye precursors of the developer (X1) and coupler type (X2), natural and synthetic direct dyes (Y) and precursors of nature-analogous dyes, such as indole and indoline derivatives, and mixtures of representatives of one or more of these groups may be used as such.

Conventionally used oxidation dye precursors of the developer type (X1) are primary aromatic amines with a further free or substituted hydroxy or amino group located in para- or ortho-position, diaminopyridine derivatives, heterocyclic hydrazones, 4-aminopyrazole derivatives and 2,4,5,6-tetraminopyrimidine and the derivatives thereof. Suitable developer components are for example p-phenylenediamine, p-tolylenediamine, p-aminophenol, o-aminophenol, 1-(2′-hydroxyethyl)-2,5-diaminobenzene, N,N-bis-(2′-hydroxyethyl)-p-phenylenediamine, 2-(2,5-diaminophenoxy)ethanol, 4-amino-3-methylphenol, 2,4,5,6-tetraminopyrimidine, 2-hydroxy-4,5,6-triaminopyrimidine, 4-hydroxy-2,5,6-triaminopyrimidine, 2,4-dihydroxy-5,6-diaminopyrimidine, 2-dimethylamino-4,5,6-triaminopyrimidine, 2-hydroxy-methylamino-4-aminophenol, bis-(4-aminophenyl)amine, 4-amino-3-fluorophenyl, 2-aminomethyl-4-aminophenol, 2-hydroxymethyl-4-aminophenol, 4-amino-2-((diethylamino)-methyl)-phenol, bis-(2-hydroxy-5-aminophenyl)methane, 1,4-bis-(4-aminophenol)-diazacycloheptane, 1,3-bis(N(2-hydroxyethyl)-N(4-aminophenylamino))-2-propanol, 4-amino-2-(2-hydroxyethoxy)phenol, 1,10-bis-(2,5-diaminophenyl)-1,4,7,10-tetraoxadecane and 4,5-diaminopyrazole derivatives according to EP 0 740 741 or WO 94/08970, such as for example 4,5-diamino-1-(2′-hydroxyethyl)-pyrazole. Particularly advantageous developer components are p-phenylenediamine, p-tolylenediamine, p-aminophenol, 1-(2′-hydroxyethyl)-2,5-diaminobenzene, 4-amino-3-methylphenol, 2-aminomethyl-4-aminophenol, 2,4,5,6-tetraminopyrimidine, 2-hydroxy-4,5,6-triaminopyrimidine, 4-hydroxy-2,5,6-triaminopyrimidine.

Oxidation dye precursors of the coupler type (X2) which are used are generally m-phenylenediamine derivatives, naphthols, resorcinol and resorcinol derivatives, pyrazolones and m-aminophenol derivatives. Examples of such coupler components are m-aminophenol and the derivatives thereof such as for example 5-amino-2-methylphenol, 5-(3-hydroxypropylamino-)2-methylphenol, 3-amino-2-chloro-6-methylphenol, 2-hydroxy-4-aminophenoxy-ethanol, 2,6-dimethyl-3-aminophenol, 3-trifluoroacetylamino-2-chloro-6-methylphenol, 5-amino-4-chloro-2-methylphenol, 5-amino-4-methoxy-2-methylphenol, 5-(2′-hydroxyethyl)-amino-2-methylphenol, 3-(diethylamino)-phenol, N-cyclopentyl-3-aminophenol, 1,3-dihydroxy-5-(methylamino)-benzene, 3-(ethylamino-4-methylphenol and 2,4-dichloro-3-aminophenol, o-aminophenol and the derivatives thereof, m-diaminobenzene and the derivatives thereof such as for example 2,4-diaminophenoxyethanol, 1,3-bis-(2,4-diaminophenoxy)propane, 1-methoxy-2-amino-4-(2′-hydroxyethylamino)-benzene, 1,3-bis-(2,4-diaminophenyl)-propane, 2,6-bis-(2-hydroxyethylamino)-1-methylbenzene and 1-amino-3-bis-(2′-hydroxyethyl)-aminobenzene, o-diaminobenzene and the derivatives thereof such as for example 3,4-diaminobenzoic acid and 2,3-diamino-1-methylbenzene, di- or trihydroxybenzene derivatives such as for example resorcinol, resorcinol monomethyl ether, 2-methylresorcinol, 5-methylresorcinol, 2,5-dimethylresorcinol, 2-chlororesorcinol, 4-chlororesorcinol, pyrogallol and 1,2,4-trihydroxybenzene, pyridine derivatives such as for example 2,6-dihydroxypyridine, 2-amino-3-hydroxypyridine, 2-amino-5-chloro-3-hydroxypyridine, 3-amino-2-methylamino-6-methoxypyridine, 2,6-dihydroxy-3,4-dimethylpyridine, 2,6-dihydroxy-4-methylpyridine, 2,6-diaminopyridine, 2,3-diamino-6-methoxypyridine and 3,5-diamino-2,6-dimethoxypyridine, naphthalene derivatives such as for example 1-naphthol, 2-methyl-1-naphthol, 2-hydroxymethyl-1-naphthol, 2-hydroxyethyl-1-naphthol, 1,5-dihydroxynaphthalene, 1,6-dihydroxynaphthalene, 1,7-dihydroxynaphthalene, 1,8-dihydroxynaphthalene, 2,7-dihydroxynaphthalene and 2,3-dihydroxynaphthalene, morpholine derivatives such as for example 6-hydroxybenzomorpholine and 6-aminobenzomorpholine, quinoxaline derivatives such as for example 6-methyl-1,2,3,4-tetrahydroquinoxaline, pyrazole derivatives such as for example 1-phenyl-3-methylpyrazol-5-one, indole derivatives such as for example 4-hydroxyindole, 6-hydroxyindole and 7-hydroxyindole, methylenedioxybenzene derivatives such as for example 1-hydroxy-3,4-methylenedioxybenzene, 1-amino-3,4-methylenedioxybenzene and 1-(2′-hydroxyethyl)-amino-3,4-methylenedioxybenzene.

Particularly suitable coupler components are 1-naphthol, 1,5-, 2,7- and 1,7-dihydroxynaphthalene, 3-aminophenol, 5-amino-2-methylphenol, 2-amino-3-hydroxypyridine, resorcinol, 4-chlororesorcinol, 2-chloro-6-methyl-3-aminophenol, 2-methylresorcinol, 5-methylresorcinol, 2,5-dimethylresorcinol and 2,6-dihydroxy-3,4-dimethylpyridine.

Direct dyes are conventionally nitrophenylenediamines, nitroaminophenols, azo dyes, anthraquinones or indophenols. Particularly suitable direct dyes are the compounds known by the international names or tradenames HC Yellow 2, HC Yellow 4, HC Yellow 5, HC Yellow 6, Basic Yellow 57, Disperse Orange 3, HC Red 3, HC Red BN, Basic Red 76, HC Blue 2, HC Blue 12, Disperse Blue 3, Basic Blue 99, HC Violet 1, Disperse Violet 1, Disperse Violet 4, Disperse Black 9, Basic Brown 16 and Basic Brown 17 and 1,4-bis-(β-hydroxyethyl)-amino-2-nitrobenzene, 4-amino-2-nitrodiphenylamine-2′-carboxylic acid, 6-nitro-1,2,3,4-tetrahydroquinoxaline, hydroxyethyl-2-nitrotoluidine, picramic acid, 2-amino-6-chloro-4-nitrophenol, 4-ethylamino-3-nitrobenzoic acid and 2-chloro-6-ethylamino-1-hydroxy-4-nitrobenzene.

Naturally occurring direct dyes contain for example henna red, henna neutral, chamomile flowers, sandalwood, black tea, alder buckthorn bark, sage, logwood, madder root, catechu, lotus tree and alkanet root.

It is not necessary for the oxidation dye precursors or the direct dyes in each case to be uniform compounds. Instead, as a result of the production processes for the individual dyes, the hair colorants according to the invention may contain subordinate quantities of still further components, provided that these do not have a disadvantageous effect on the dyeing result or have to be excluded for other, for example toxicological, reasons.

Precursors of nature-analogous dyes which are used are for example indoles and indolines and the physiologically acceptable salts thereof. Preferably those indoles and indolines are used which comprise at least one hydroxyl or amino group, preferably as a substituent on the six-membered ring. These groups may bear further substituents, for example in the form of etherification or esterification of the hydroxyl group or alkylation of the amino group. Particularly advantageous properties are exhibited by 5,6-dihydroxyindoline, N-methyl-5,6-dihydroxyindoline, N-ethyl-5,6-dihydroxyindoline, N-propyl-5,6-dihydroxyindoline, N-butyl-5,6-dihydroxyindoline, 5,6-dihydroxyindoline-2-carboxylic acid, 6-hydroxyindoline, 6-aminoindoline and 4-aminoindoline and 5,6-dihydroxyindole, N-methyl-5,6-dihydroxyindole, N-ethyl-5,6-dihydroxyindole, N-propyl-5,6-dihydroxyindole, N-butyl-5,6-dihydroxyindole, 5,6-dihydroxyindole-2-carboxylic acid, 6-hydroxyindole, 6-aminoindole and 4-aminoindole.

Within this group, particular emphasis should be placed on N-methyl-5,6-dihydroxyindoline, N-ethyl-5,6-dihydroxyindoline, N-propyl-5,6-dihydroxyindoline, N-butyl-5,6-dihydroxyindoline, and in particular 5,6-dihydroxyindoline, and N-methyl-5,6-dihydroxyindole, N-ethyl-5,6-dihydroxyindole, N-propyl-5,6-dihydroxyindole, N-butyl-5,6-dihydroxyindole, and in particular 5,6-dihydroxyindole.

In the dyes used in the context of the method according to the invention, the indoline and indole derivatives may be used both as free bases and in the form of the physiologically acceptable salts thereof with inorganic or organic acids, for example hydrochlorides, sulfates and hydrobromides.

When using dye precursors of the indoline or indole type, it may be preferable to use these together with at least one amino acid and/or at least one oligopeptide. Preferred amino acids are aminocarboxylic acids, in particular α-aminocarboxylic acids and ω-aminocarboxylic acids. Among α-aminocarboxylic acids, arginine, lysine, ornithine and histidine are in turn particularly preferred. One very particularly preferred amino acid is arginine, which is used in particular in free form, but also as the hydrochloride.

Both the oxidation dye precursors and the direct dyes and the precursors of nature-analogous dyes are preferably present in the agents according to the invention in quantities of 0.01 to 20 wt. %, preferably of 0.1 to 5 wt. %, in each case relative to the total agent.

The advantage which is achieved by the composition according to the invention in conjunction with the dye precursors is distinctly improved deposition of the dye precursors on the hair. In addition to increased deposition on the hair, the composition according to the invention also brings about more rapid penetration into the hair. The desired hair color is furthermore developed more quickly. The application time of the composition may be shortened by at least 10% while achieving an identical dyeing result. Using the combination according to the invention, the application time can be shortened by up to 40% while achieving an identical dyeing result. All of these effects are achieved with simultaneously enhanced washing resistance of the hair color once it has developed. The invention includes the teaching that, on the other hand, on the basis of the effects achieved the dye concentration may also be distinctly reduced. On the one hand, this is of great economic significance, while, on the other hand, this also means a considerable improvement of the dermatological compatibility of the entire composition.

One very particularly preferred composition of the invention therefore relates to cosmetic agents for dyeing skin and hair containing the compositions according to the invention and a dye precursor, and to the use of this agent and to a method for hair dyeing or for reviving hair dyeing with this agent.

Hair dyes, in particular if dyeing proceeds by oxidation, whether with atmospheric oxygen or other oxidizing agents such as hydrogen peroxide, are conventionally adjusted to weakly acidic to alkaline, i.e. to pH values in the range from approx. 5 to 11. For this purpose, the dyes contain alkalizing agents, conventionally alkali metal or alkaline earth metal hydroxides, ammonia or organic amines. Preferred alkalizing agents are monoethanolamine, monoisopropanolamine, 2-amino-2-methylpropanol, 2-amino-2-methyl-1,3-propanediol, 2-amino-2-ethyl-1,3-propanediol, 2-amino-2-methylbutanol and triethanolamine and alkali metal and alkaline earth metal hydroxides. In particular monoethanolamine, triethanolamine and 2-amino-2-methylpropanol and 2-amino-2-methyl-1,3-propanediol are preferred in the context of this group. ω-Amino acids such as ω-aminocaproic acid may also be used as alkalizing agents.

If the actual hair colors are formed in the context of an oxidative process, conventional oxidizing agents, such as in particular hydrogen peroxide or the addition products thereof onto urea, melamine or sodium borate may be used. Oxidation with atmospheric oxygen as the sole oxidizing agent may however be preferred. It is furthermore possible to carry out oxidation with the assistance of enzymes, the enzymes being used both for producing oxidizing per compounds and for enhancing the action of a small quantity of an oxidizing agent which is present, or also enzymes which transfer the electrons from suitable developer components (reducing agents) to atmospheric oxygen. Preferred enzymes are here not only oxidases such as tyrosinase, ascorbate oxidase and laccase but also glucose oxidase, uricase or pyruvate oxidase. The approach of enhancing the action of small quantities (for example 1% and less, relative to the total agent) of hydrogen peroxide by peroxidases may also be mentioned.

The oxidizing agent preparation is then conveniently mixed with the dye precursor preparation immediately before the hair is dyed. The resultant ready-to-use hair dye preparation should preferably have a pH value in the range from 6 to 10. It is particularly preferable to apply the hair dye in a weakly alkaline medium. Application temperatures may be in a range of between 15 and 40° C., preferably at scalp temperature. After an exposure time of approx. 5 to 45, in particular 15 to 30, minutes, the hair dye is rinsed out of the hair to be dyed. Rewashing with a shampoo is not required if a carrier with an elevated surfactant content, e.g. a coloring shampoo, has been used.

In the case in particular of hair which is difficult to dye, however, the preparation with the dye precursors may also be applied onto the hair without prior mixing with the oxidation component. After an exposure time of 20 to 30 minutes, the oxidation component is then applied, optionally after intermediate rinsing. After a further exposure time of 10 to 20 minutes, the hair is then rinsed and reshampooed if desired. According to a first variant of this embodiment, in which prior application of the dye precursors is intended to bring about better penetration into the hair, the corresponding agent is adjusted to a pH value of approx. 4 to 7. According to a second variant, atmospheric oxidation is initially sought, the applied agent preferably having a pH value of 7 to 10. For the subsequent accelerated post-oxidation, the use of acidic peroxydisulfate solutions as oxidizing agent may be preferred.

Dye formation may furthermore be supported and enhanced by adding specific metal ions to the agent. Such metal ions are for example Zn²⁺, Cu²⁺, Fe²⁺, Fe³⁺, Mn²⁺, Mn⁴⁺, Li⁺, Mg²⁺, Ca²⁺ and Al³⁺. Zn²⁺, Cu²⁺ and Mn²⁺ are here particularly suitable. The metal ions may in principle be used in the form of any desired, physiologically acceptable salt. Preferred salts are acetates, sulfates, halides, lactates and tartrates. By using these metal salts, it is possible both to accelerate dye development and to have a targeted influence on color shade.

It may additionally prove advantageous and still further increase the synergistic effects of the compositions according to the invention if penetration aids and/or swelling agents (M) are present. These substances may bring about better penetration of active ingredients into the skin or hair to be treated. Examples of these include urea and urea derivatives, guanidine and the derivatives thereof, arginine and the derivatives thereof, water glass, imidazole and the derivatives thereof, histidine and the derivatives thereof, benzyl alcohol, glycerol, glycol and glycol ethers, propylene glycol and propylene glycol ethers, for example propylene glycol monoethyl ether, carbonates, hydrogencarbonates, diols and triols, and in particular 1,2-diols and 1,3-diols such as for example 1,2-propanediol, 1,2-pentanediol, 1,2-hexanediol, 1,2-dodecanediol, 1,3-propanediol, 1,6-hexanediol, 1,5-pentanediol, 1,4-butanediol.

Dyes which may be used for coloring the compositions are those approved substances suitable for cosmetic purposes, as are for example compiled in the publication “Kosmetische Färbemittel” [cosmetic dyestuffs] from the dyestuffs committee of DFG, the German Research Foundation, Verlag Chemie, Weinheim, 1984, pages 81-106. These dyes are conventionally used in concentrations of from 0.001 to 0.1 wt. %, relative to the total mixture.

The pH value of the preparations according to the invention may in principle range from 2-11. Depending on the purpose and the use of the composition according to the invention, the pH value is very purposefully selected and adjusted. For dye preparations, it is preferably between 5 and 11, for example, with values of 6 to 10 being particularly preferred. For cleansing compositions, it is for example between 4 and 7.5, preferably between 4 and 6.

This pH value may be established by using virtually any acid or base which is usable for cosmetic purposes. Preferred bases are ammonia, alkali metal hydroxides, monoethanolamine, triethanolamine and N,N,N′,N′-tetrakis-(2-hydroxypropyl)-ethylenediamine.

Edible acids are conventionally used as acids. Edible acids are taken to mean such acids which are consumed within the context of conventional food intake and have a positive effect on the human body. Edible acids are for example acetic acid, lactic acid, tartaric acid, citric acid, malic acid, ascorbic acid and gluconic acid. It is particularly preferred for the purposes of the invention to use citric acid and lactic acid.

It has furthermore been found that the action of the active ingredient according to the invention in the agents according to the invention may be further enhanced in combination with substances which contain primary or secondary amino groups. Examples of such amino compounds which may be mentioned are ammonia, monoethanolamine, 2-amino-2-methyl-1-propanol, 2-amino-2-methylpropanediol and basic amino acids such as for example lysine, arginine or histidine. It goes without saying that these amines may also be used in the form of the corresponding salts with inorganic and/or organic acids, such as for example as ammonium carbonate, ammonium citrate, ammonium oxalate, ammonium tartrate or lysine hydrochloride. The amines are used together with the active ingredient according to the invention in ratios of 1:10 to 10:1, preferably of 3:1 to 1:3, and very particularly preferably in stoichiometric quantities.

Protic solvents, such as for example water, and alcohols may be present in the compositions according to the invention. Alcohols which are used include all physiologically safely usable alcohols, for example methanol, ethanol, isopropanol, propanol, butanol, isobutanol, glycol, glycerol and mixtures thereof with one another. The proportion of protic solvents in every case makes up the composition according to the invention to 100 parts by weight. The cosmetic compositions preferably contain at least 30 wt. % protic solvents, particularly preferably at least 50 wt. % and very particularly preferably at least 75 wt. % and very highly preferably at least 85 wt. % protic solvents.

UV filters (I) may furthermore be used in one very particularly preferred embodiment of the invention. UV filters to be used according to the invention are not subject to any general restrictions with regard to structure and physical properties. Rather, any UV filters usable in the field of cosmetics whose absorption maximum is in the UVA (315-400 nm), the UVB (280-315 nm) or the UVC (<280 nm) range are suitable. UV filters with an absorption maximum in the UVB range, in particular in the range from approx. 280 to approx. 300 nm, are particularly preferred.

The UV filters used according to the invention may for example be selected from substituted benzophenones, p-aminobenzoates, diphenylacrylates, cinnamates, salicylates, benzimidazoles and o-aminobenzoates.

Examples of UV filters usable according to the invention are 4-aminobenzoic acid, N,N,N-trimethyl-4-(2-oxoborn-3-ylidenemethyl)aniline methyl sulfate, 3,3,5-trimethylcyclohexyl salicylate (homosalate), 2-hydroxy-4-methoxybenzophenone (benzophenone-3; Uvinul® M 40, Uvasorb® MET, Neo Heliopan® BB, Eusolex® 4360), 2-phenylbenzimidazole-5-sulfonic acid and the potassium, sodium and triethanolamine salts thereof (phenylbenzimidazolesulfonic acid; Parsol® HS; Neo Heliopan® Hydro), 3,3′-(1,4-phenylenedimethylene)-bis(7,7-dimethyl-2-oxobicyclo-[2.2.1]hept-1-ylmethanesulfonic acid) and the salts thereof, 1-(4-tert.-butylphenyl)-3-(4-methoxyphenyl)-propane-1,3-dione (butyl methoxydibenzoylmethane; Parsol® 1789, Eusolex® 9020), α-(2-oxoborn-3-ylidene)-toluene-4-sulfonic acid and the salts thereof, ethoxylated 4-aminobenzoic acid ethyl ester (PEG-25 PABA; Uvinul® P 25), 4-dimethylaminobenzoic acid 2-ethylhexyl ester (octyl dimethyl PABA; Uvasorb® DMO, Escalol® 507, Eusolex® 6007), salicylic acid 2-ethylhexyl ester (octyl salicylate; Escalol® 587, Neo Heliopan® OS, Uvinul® 018), 4-methoxycinnamic acid isopentyl ester (isoamyl p-methoxycinnamate; Neo Heliopan® E 1000), 4-methoxycinnamic acid 2-ethylhexyl ester (octyl methoxycinnamate; Parsol® MCX, Escalol® 557, Neo Heliopan® AV), 2-hydroxy-4-methoxybenzophenone-5-sulfonic acid and the sodium salt thereof (benzophenone-4; Uvinul® MS 40; Uvasorb® S 5), 3-(4′-methylbenzylidene)-D,L-camphor (4-methylbenzylidene camphor; Parsol® 5000, Eusolex® 6300), 3-benzylidenecamphor (3-benzylidene camphor), 4-isopropylbenzyl salicylate, 2,4,6-trianilino-(p-carbo-2′-ethylhexyl-1′-oxy)-1,3,5-triazine, 3-imidazol-4-ylacrylic acid and the ethyl esters thereof, polymers of N-{(2 and 4)-[2-oxoborn-3-ylidenemethyl]benzyl}-acrylamide, 2,4-dihydroxybenzophenone (benzophenone-1; Uvasorb® 20 H, Uvinul® 400), 1,1′-diphenylacrylonitrile acid 2-ethylhexyl ester (octocrylene; Eusolex® OCR, Neo Heliopan® Type 303, Uvinul® N 539 SG), o-aminobenzoic acid menthyl ester (menthyl anthranilate; Neo Heliopan® MA), 2,2′,4,4′-tetrahydroxybenzophenone (benzophenone-2; Uvinul® D-50), 2,2′-dihydroxy-4,4′-dimethoxybenzophenone (benzophenone-6), 2,2′-dihydroxy-4,4′-dimethoxybenzophenone-5-sodium sulfonate and 2-cyano-3,3-diphenylacryl acid 2′-ethylhexyl ester. Preference is given to 4-aminobenzoic acid, N,N,N-trimethyl-4-(2-oxoborn-3-ylidenemethyl)aniline methyl sulfate, 3,3,5-trimethyl cyclohexyl salicylate, 2-hydroxy-4-methoxybenzophenone, 2-phenylbenzimidazole-5-sulfonic acid and the potassium, sodium and triethanolamine salts thereof, 3,3′-(1,4-phenylenedimethylene)-bis(7,7-dimethyl-2-oxobicyclo-[2.2.1]hept-1-ylmethanesulfonic acid) and the salts thereof, 1-(4-tert.-butylphenyl)-3-(4-methoxyphenyl)propane-1,3-dione, α-(2-oxoborn-3-ylidene)-toluene-4-sulfonic acid and the salts thereof, ethoxylated 4-aminobenzoic acid ethyl ester, 4-dimethylaminobenzoic acid 2-ethylhexyl ester, salicylic acid 2-ethylhexyl ester, 4-methoxycinnamic acid isopentyl ester, 4-methoxycinnamic acid 2-ethylhexyl ester, 2-hydroxy-4-methoxybenzophenone 5-sulfonic acid and the sodium salt thereof, 3-(4′-methylbenzylidene)-D,L-camphor, 3-benzylidenecamphor, 4-isopropylbenzyl salicylate, 2,4,6-trianilino-(p-carbo-2′-ethylhexyl-1′-oxy)-1,3,5-triazine, 3-imidazol-4-ylacrylic acid and the ethyl esters thereof, polymers of N-{(2 and 4)-[2-oxoborn-3-ylidenemethyl]benzyl}-acrylamide. Very particular preference is given according to the invention to 2-hydroxy-4-methoxybenzophenone, 2-phenylbenzimidazole-5-sulfonic acid and the potassium, sodium and triethanolamine salts thereof, 1-(4-tert.-butylphenyl)-3-(4-methoxyphenyl)-propane-1,3-dione, 4-methoxycinnamic acid 2-ethylhexyl ester and 3-(4′-methylbenzylidene)-D, L-camphor.

Those UV filters are preferred whose molar extinction coefficient at the absorption maximum is above 15,000, in particular above 20,000.

It has additionally been found that, in the case of structurally similar UV filters, in many cases the water-insoluble compound displays for the purposes of the teaching according to the invention the greater action relative to those water-soluble compounds which differ therefrom by one or more additionally ionic groups. Those UV filters which are understood for the purposes of the invention to be water-insoluble are those which at 20° C. are only 1 wt. %, in particular no more than 0.1 wt. %, soluble in water. Furthermore, these compounds should be at least 0.1, in particular at least 1 wt. %, soluble in conventional cosmetic oil components at room temperature. The use of water-insoluble UV filters may therefore be preferred according to the invention.

According to a further embodiment of the invention, preferred UV-filters are those which comprise a cationic group, in particular a quaternary ammonium group.

These UV filters have the general structure U-Q.

The structural element U therein denotes a UV radiation-absorbing group. This group may be derived in principle from the known above-stated UV filters usable in the field of cosmetics, in which a group, generally a hydrogen atom, of the UV filter is replaced by a cationic group Q, in particular with a quaternary amino function.

Compounds from which the structural element U may be derived are for example

substituted benzophenones,
p-aminobenzoates,
diphenylacrylates,
cinnamates,
salicylates,
benzimidazoles and
o-aminobenzoic acid esters.

Structural elements U which are derived from cinnamic acid amide or from N,N-dimethylaminobenzoic acid amide are preferred according to the invention.

The structural elements U may in principle be selected such that the absorption maximum of the UV filters may lie both in the UVA (315-400 nm) and in the UVB (280-315 nm) or in the UVC (<280 nm) range. UV filters with an absorption maximum in the UVB range, in particular in the range from approx. 280 to approx. 300 nm, are particularly preferred.

Furthermore, the structural element U, also as a function of structural element Q, is preferably selected such that the molar extinction coefficient of the UV filter at the absorption maximum is above 15000, in particular above 20000.

The structural element Q preferably contains a quaternary ammonium group as the cationic group. This quaternary ammonium group may in principle be linked directly to the structural element U, such that the structural element U is one of the four substituents of the positively charged nitrogen atom. However, one of the four substituents on the positively charged nitrogen atom is preferably a group, in particular an alkylene group with 2 to 6 carbon atoms, which functions as a link between the structural element U and the positively charged nitrogen atom.

Advantageously, the group Q has the general structure —(CH₂)ₓ—N⁺R¹R²R³ X⁻, in which x denotes an integer from 1 to 4, R¹ and R² mutually independently denote C₁₋₄ alkyl groups, R³ denotes a C₁₋₂₂ alkyl group or a benzyl group and X⁻ denotes a physiologically acceptable anion. In the context of this general structure, x preferably denotes the number 3, R¹ and R² in each case denote a methyl group and R³ denotes either a methyl group or a saturated or unsaturated, linear or branched, hydrocarbon chain with 8 to 22, in particular 10 to 18, carbon atoms.

Physiologically acceptable anions are for example inorganic anions such as halides, in particular chloride, bromide and fluoride, sulfate ions and phosphate ions and organic anions such as lactate, citrate, acetate, tartrate, methosulfate and tosylate.

Two preferred UV filters with cationic groups are the commercially obtainable compounds cinnamic acid amidopropyltrimethylammonium chloride (Incroquat® UV-283) and dodecyldimethylaminobenzamidopropyldimethylammonium tosylate (Escalol® HP 610).

It goes without saying that the teaching according to the invention also includes the use of a combination of two or more UV filters. In the context of this embodiment, the combination of at least one water-insoluble UV filter with at least one UV filter having a cationic group is preferred.

The UV filters (I) are conventionally contained in the agents used according to the invention in quantities of 0.1-5 wt. % relative to the total agent. Quantities of 0.4-2.5 wt. % are preferred.

Further active ingredients and auxiliary substances and additives are for example

thickeners such as agar-agar, guar gum, alginates, xanthan gum, gum arabic, karaya gum, locust bean flour, linseed gums, dextrans, cellulose derivatives, for example methylcellulose, hydroxyalkylcellulose and carboxymethylcellulose, starch fractions and derivatives such as amylose, amylopectin and dextrins, clays such as for example bentonite or completely synthetic hydrocolloids such as for example polyvinyl alcohol,
hair-conditioning compounds such as phospholipids, for example soy lecithin, egg lecithin and cephalins,
dimethyl isosorbide and cyclodextrins,
symmetrical and asymmetrical, linear and branched dialkyl ethers having a total of between 12 to 36 C atoms, in particular 12 to 24 C atoms, such as for example di-n-octyl ether, di-n-decyl ether, di-n-nonyl ether, di-n-undecyl ether and di-n-dodecyl ether, n-hexyl-n-octyl ether, n-octyl-n-decyl ether, n-decyl-n-undecyl ether, n-undecyl-n-dodecyl ether and n-hexyl-n-undecyl ether and di-tert.-butyl ether, di-iso-pentyl ether, di-3-ethyldecyl ether, tert.-butyl-n-octyl ether, iso-pentyl-n-octyl ether and 2-methylpentyl-n-octyl ether,
active ingredients which improve fiber structure, in particular mono-, di- and oligosaccharides such as for example glucose, galactose, fructose, fruit sugars and lactose,
phospholipids, for example soy lecithin, egg lecithin and cephalins,
quaternized amines such as methyl-1-alkylamidoethyl-2-alkylimidazolinium methosulfate,
antidandruff active ingredients such as piroctone olamine, zinc omadine and climbazole,
active ingredients such as allantoin and bisabolol,
cholesterol,
complexing agents such as EDTA, NTA, β-alaninediacetic acid, iminodisuccinic acid and the salts thereof, etidronic acid and the salts thereof and phosphonic acids,
swelling and penetrating substances such as primary, secondary and tertiary phosphates
opacifiers such as latex, styrene/PVP and styrene/acrylamide copolymers
pearlescent agents such as ethylene glycol mono- and distearate as well as PEG-3 distearate,
pigments,
reducing agents such as for example thioglycolic acid and the derivatives thereof, thiolactic acid, cysteamine, thiomalic acid and α-mercaptoethanesulfonic acid,
antioxidants.

With regard to further optional components and the quantities of these components used, reference is explicitly made to the relevant handbooks known to a person skilled in the art, for example the monograph by K. H. Schräder.

The composition according to the invention is preferably formulated in an aqueous, an alcoholic or in an aqueous/alcoholic medium preferably comprising at least 10 weight percent water. Alcohols which may be present are in particular the lower alcohols with 1 to 4 carbon atoms such as for example ethanol and isopropanol which are conventionally used for cosmetic purposes. The agent according to the invention may be present in a pH range from 2 to 11. The pH range between 2 and 8 is particularly preferred.

Additional cosolvents which may be present are organic solvents or a mixture of solvents with a boiling point of below 400° C. in a quantity of 0.1 to 15 weight percent, preferably of 1 to 10 weight percent. Particularly suitable additional cosolvents are unbranched or branched hydrocarbons such as pentane, hexane, isopentane and cyclic hydrocarbons such as cyclopentane and cyclohexane. Further, particularly preferred water-soluble solvents are glycerol, ethylene glycol and propylene glycol in a quantity of up to 30 weight percent.

The present invention also provides a method for hair treatment in which the composition according to the invention is worked into the moist hair and, after an exposure time of from a few seconds up to 5 minutes, the composition is rinsed out again with water which is cold or up to approx. 40° C. in temperature, the hair is optionally dried with a hand towel, then a further caring and conditioning composition, preferably likewise according to the invention but also a conditioning composition not according to the invention, is worked into the towel-dry to wet hair, this composition remaining on the hair for from a few seconds up to 30 minutes, and is then rinsed out, the hair is again dried with a hand towel, optionally followed by a blow drying step in order to dry the hair completely, and finally a styling and dressing composition C which is essential according to the invention is placed in the hair and is preferably not rinsed out again. It goes without saying that both composition B as a caring and conditioning composition and composition C may in turn contain the composition according to the invention. It is preferred for at least composition C likewise to contain the composition according to the invention.

The method according to the invention also provides that the first hair washing step may optionally be repeated.

Any types of packaging known to a person skilled in the art may in principle be used as packaging for the cosmetic compositions of the present invention. These are in particular pots, tubes, bottles, sachets. Various configurations are possible with regard to shape and color. It is preferred, however, for the external packaging surface of the packaging to comprise a transparent proportion of at least 15%. Still more suitable, in order to show the attractive appearance off to best advantage, is a transparent surface proportion of at least 30%, but in particular of 50%, while a transparent surface proportion of at least 75% is excellent for this purpose and one of at least 85% is outstandingly good.

For the purposes of the invention, transparency is taken to mean that the composition according to the invention transmits visible light. The transmittance of the packaging itself is measured for this purpose. This is measured with visible light in a conventional measuring arrangement. For the purposes of the present invention, transparency is deemed to exist from a visible light transmittance of at least 20%. For the purposes of the invention, clarity is deemed to exist from a transmittance of at least 60%. Transmittance of at least 40% is preferred according to the invention. Transmittance of at least 60% is particularly preferred, transmittance of visible light of at least 75% being very particularly preferred. And transmittance of at least 85% is very highly preferred.

In one development according to the invention, the compositions according to the invention may consist of two discrete, mutually independent phases or even of two entirely discrete mutually independent compositions in a common outer packaging. In the latter case, the composition according to the invention may either be in one of the two compositions or in each of the compositions or it may not be obtained until the compositions, which are kept apart from one another until use, have been mixed. In these cases, the separately produced compositions are also in each case separately packaged in separate containers. Containers which may here be considered are any conventional packaging containers known to a person skilled in the art and conventional for hair treatment agents, such as tubes, bottles or pots. In a further development according to the invention one of the compositions may here be filled in the stated packaging in such a manner that it may readily be removed therefrom in measured portions of at least 0.5 up to 20 g. This may be achieved for example in the case a squeezable tube by the outside of the tube being marked in 0.5 ml graduations. These graduations then make it possible to remove defined volumes of the basic composition. In the case of a pot, for example an accompanying spoon with a defined volume of for instance 0.5 ml to 5.0 ml makes it possible to remove defined quantities.

Within this latter configuration, a composition may very particularly preferably be packaged in a package from which the composition can be removed in an accurately dispensed volume. The accuracy of volume dispensing here amounts to at least 0.1 ml. Dispensing accuracy is particularly preferably 0.25 ml and very particularly preferably at least 0.5 ml. One example of such a dispensing package which may be mentioned is a bottle, tube or pot with an additional dropper. It goes without saying that it is also possible to package this composition in a small container made of glass or plastics material and, in a manner similar to medical packaging for ear drops, to close it with a closure incorporating a dropper. Alternatively, a dispensing dropper may also be enclosed with the package. In a further feature of this embodiment, for example, the lid of the package of the basic composition is constructed such that the appropriate volumes of the two compositions can be mixed together in said lid. To this end, in a preferred embodiment, the lid may be curved and be capable of accommodating volumes of at least 5 to 50 ml. Mixing and subsequent application of the ready-to-use mixture of the two compositions may furthermore be facilitated with a paintbrush or spatula. All the necessary packages are then offered for sale in a common outer packaging.

In a further development according to the invention, the compositions may be packaged in an appropriate 2 chamber package. Such packages are commercially available. The compositions could then be removed separately and in variable quantities from the respective chambers and mixed with one another in a separate container. At the latest after mixing, this mixed composition contains the composition according to the invention of a mild anionic surfactant and a cationic and/or amphoteric polymer.

In one particularly preferred embodiment, a two-chamber tube may for example be used. Such tubes are described, for example, in DE 102004009424. In such tubes, the volume of the products to be removed from the individual containers may be varied. The two products are then mixed either in a separate mixing container or in the hand immediately before use.

In one further particularly preferred embodiment, a two-chamber container consisting of two metering dispensers is used. Each metering dispenser may be arranged separately and independently of the other dispenser with regard to the pump system such that the quantities of product to be removed from the respective containers may be varied. This may be achieved, for example, by two cartridges, each of which accommodates an air-tight trailing piston. Mixing of the two compositions may here ideally proceed in the dispenser head. Such packages are commercially offered for sale for example by DIALPACK GmbH.

A feature common to all embodiments with two discrete phases separate from one another which contain the composition according to the invention at the latest once these two discrete phases have been mixed is that the outer packaging contains all the individual components, detailed instructions for use for establishing the initial condition of the hair and, based thereon, a recommendation for the optimum mixing ratio of the compositions.

One presentation which is outstandingly suitable according to the invention is a spray presentation of the composition according to the invention.

A spray presentation of the composition according to the invention is such that it permits atomization of the composition. The composition according to the invention may for example be presented as an aerosol, as a non-aerosol spray lotion, which is used by being atomized by means of a mechanical device, as an aerosol foam or as a non-aerosol foam, which is provided in combination with a suitable mechanical device for foaming the composition.

One suitable application form is an aerosol and/or non-aerosol spray-applied preparation. In this case the composition according to the invention is atomized with the assistance of a suitable mechanically operated spray device. Mechanical spray devices should be taken to mean such devices which permit the atomization of a liquid without using a propellant. One suitable mechanical spray device which may be used is, for example, a spray pump or a resilient container provided with a spray valve, in which the cosmetic agent according to the invention is packaged under pressure resulting in expansion of the resilient container and from which the agent is continuously released on opening the spray valve as a result of contraction of the resilient container.

If the composition according to the invention assumes the form of a hair mousse, it contains at least one conventional foam-forming substance which is known for this purpose. The agent is foamed with or without the assistance of propellant gases or chemical propellants and worked into the hair as a mousse and not rinsed out, so remaining in the hair. A hair mousse according to the invention comprises as an additional component a chemical propellant and/or a mechanical device for foaming the composition. Mechanical foaming devices should be taken to mean such devices which permit foaming of a liquid with or without using a propellant. A suitable mechanical foaming device which may be used is, for example, a conventional commercial pump foamer or an aerosol foaming head.

The development of the invention as aerosol application may be achieved by using any appropriate aerosol valve which permits the spraying rate which is preferred according to the invention and the corresponding droplet size. It may here be advantageous for the valve orifice to have a diameter of at most 0.4 mm. An orifice of 0.35 mm is here preferred. Valve orifices of at most 0.3 mm are very particularly preferred. Corresponding aerosol valves are described, for example, in patents U.S. Pat. No. 4,152,416, U.S. Pat. No. 3,083,917, U.S. Pat. No. 3,083,918, and U.S. Pat. No. 3,544,258. Such valves may be purchased commercially for example from Seaquist Perfect Dispensing GmbH or Coster Technologie Speciali S.p.A. In one very particularly preferred embodiment, the valve used is an Ariane M type valve from Seaquist. It may here be particularly preferred for this valve to be used together with a specific throttle device. The throttle device is here located either in the valve stem or in the spray head. Another particularly preferred embodiment uses a valve with a side bore as the valve, as is for example offered for sale by Coster under model number K 125 SL 184/3/6.

With regard to further development, explicit reference is made to Andreas Domsch, “Die kosmetischen Präparate” [cosmetic preparations], volume II, chapter 4, Aerosols, pages 259 et seq., Verlag für die chemische Industrie, H. Ziolkowsky K G, Augsburg, 1992. It goes without saying that the invention also encompasses aerosol containers which may be made not only from aluminum monobloc cans, but also from plastics such as PET or glass.

The development of the invention as a non-aerosol may be achieved by using any spray pump which permits the spraying rate according to the invention. Corresponding systems are for example commercially available under the name Calmar Mark II from Calmar Inc.

Propellant gases must be used in order to use the compositions according to the invention as aerosol sprays. Propellant gases which are preferred according to the invention are selected from hydrocarbons with 3 to 5 carbon atoms, such as propane, n-butane, iso-butane, n-pentane and iso-pentane, dimethyl ether, carbon dioxide, dinitrogen oxide, fluorocarbons and chlorofluorocarbons and mixtures of these substances. Very particularly preferred propellant gases are propane, butane, isobutane, pentane, isopentane, dimethyl ether and the mixtures of these above-stated propellant gases in each case with one another. Propellant gases which are most highly preferred according to the invention are mixtures of dimethyl ether with hydrocarbons. Within the group of hydrocarbon propellant gases, n-butane and propane are preferred.

The propellant is preferably selected such that it may simultaneously act as a solvent for further constituents such as for example oil and wax components, the fatty substances (D). The propellant may act as solvent for these latter-stated components if, relative to the propellant, they are at least 0.5 wt. % soluble therein at 20° C.

According to one preferred embodiment the preparations according to the invention contain the stated hydrocarbons or mixtures of the stated hydrocarbons with dimethyl ether as the sole propellant. The invention does, however, also explicitly encompass co-use of propellants of the chlorofluorocarbon type, but especially of the fluorocarbon type.

The propellant gases are present in quantities of 5-98 wt. %, preferably 10-98 wt. % and particularly preferably 20-98 wt. %, very particularly preferably of 40 to 98 wt. %, in each case relative to the total aerosol composition.

The compositions according to the invention may be packaged in conventional commercial aerosol cans. The cans may be made from tin plate or aluminum. The cans may furthermore be coated on the inside in order to keep the risk of corrosion as low as possible.

If the compositions according to the invention are used as a non-aerosol spray-applied preparation, it goes without saying that no propellant gas is present. However, the spray heads should be selected in each case on the basis of the corresponding required spray rates.

The cans are equipped with a suitable spray head. Depending on the spray head, output rates, relative to completely full cans, of 0.1 g/s to 5.0 g/s are possible. The spraying rate is here determined such that an aerosol can filled with propellant gas and the corresponding composition and closed with the appropriate valve is first of all weighed at room temperature (approx. 23° C.). The can and its contents are then shaken vigorously by hand 10 times to ensure thorough mixing of the contents. With the can upright, the valve is actuated for 10 s. Reweighing is then carried out. This procedure is carried out 5 times in succession and the statistical mean of the results is calculated. The difference between the two weighing results is the spraying rate per 10 s. The spraying rate per second can be determined by simple division. In the case of non-aerosols, the spray mechanism is correspondingly actuated 10 times. In the latter case, the spraying rate should be taken to mean the average quantity discharged per spray stroke (pump stroke). Spraying rates of 0.1 to 0.5 g/s are here preferred. Spraying rates of 0.1 to 0.4 g/s are particularly preferred.

While particular embodiments of the present invention have been illustrated and described, it would be obvious to those skilled in the art that various other changes and modifications can be made without departing from the spirit and scope of the invention.

Other than where otherwise indicated, or where required to distinguish over the prior art, all numbers expressing quantities of ingredients herein are to be understood as modified in all instances by the term “about”. As used herein, the words “may” and “may be” are to be interpreted in an open-ended, non-restrictive manner. At minimum, “may” and “may be” are to be interpreted as definitively including, but not limited to, the composition, structure, or act recited.

As used herein, and in particular as used herein to define the elements of the claims that follow, the articles “a” and “an” are synonymous and used interchangeably with “at least one” or “one or more,” disclosing or encompassing both the singular and the plural, unless specifically defined herein otherwise. The conjunction “or” is used herein in both in the conjunctive and disjunctive sense, such that phrases or terms conjoined by “or” disclose or encompass each phrase or term alone as well as any combination so conjoined, unless specifically defined herein otherwise.

The description of a group or class of materials as suitable or preferred for a given purpose in connection with the invention implies that mixtures of any two or more of the members of the group or class are equally suitable or preferred. Description of constituents in chemical terms refers unless otherwise indicated, to the constituents at the time of addition to any combination specified in the description, and does not necessarily preclude chemical interactions among the constituents of a mixture once mixed. Steps in any method disclosed or claimed need not be performed in the order recited, except as otherwise specifically disclosed or claimed.

Changes in form and substitution of equivalents are contemplated as circumstances may suggest or render expedient. Although specific terms have been employed herein, such terms are intended in a descriptive sense and not for purposes of limitation.

What is claimed:

1. A hair-conditioning agent comprising at least two different cationic polymers and a water-soluble silicone.

2. The hair-conditioning agent of claim 1, wherein at least one of the cationic polymers comprises a natural cationic polymer.

3. The hair-conditioning agent of claim 2, wherein at least one of the natural cationic polymers comprises a cationic polysaccharide.

4. The hair-conditioning agent of claim 3, wherein the natural cationic polysaccharide comprises a cationic derivative of cellulose, starch, guar, or chitosan.

5. The hair-conditioning agent of claim 1, wherein the cationic polymers comprise natural cationic polymers.

6. The hair-conditioning agent of claim 1, wherein at least two of the at least two different cationic polymers comprise natural cationic polymers.

7. The hair-conditioning agent of claim 6, wherein the at least two natural cationic polymers comprise cationic polysaccharides.

8. The hair-conditioning agent of claim 7, wherein both of the natural cationic polysaccharides comprise a cationic derivative of cellulose, starch, guar, or chitosan.

9. The hair-conditioning agent of claim 1, wherein one of the cationic polymers comprises a cationic derivative of chitosan.

10. The hair-conditioning agent of claim 1, wherein one of the cationic polymers comprises a cationic derivative of cellulose or of guar.

11. The hair-conditioning agent of claim 1, wherein one of the cationic polymers comprises a cationic derivative of chitosan and the second cationic polymer comprises a cationic derivative of guar.

12. A method of conditioning keratin fibers, comprising contacting a keratin fiber with a conditioning-effective amount of the hair-conditioning agent of claim 1.

13. A method of conditioning keratin fibers, comprising contacting a plurality of keratin fibers with an amount of the hair-conditioning agent of claim 1 effective to increase the volume of the keratin fibers on styling.

14. A method for conditioning keratin fibers, wherein the hair-conditioning agent of claim 1 is applied as a spray-applied preparation onto wet or dry keratin fibers and left on the keratin fibers until they are next washed.

15. A cosmetic preparation comprising the hair-conditioning agent of claim 1 in a transparent package suitable for dispensing the agent in the form of uniform, small droplets.
